(19) United States
(12) Patent Application Publication
(10) Pub. No.: US 2008/0027014 A1
(43) Pub. Date: Jan. 31, 2008

(54) Novel SGLT inhibitors

(75) Inventors: Sumihiro Nomura, Osaka (JP); Mitsuya Hongu, Osaka (JP)

Correspondence Address: BIRCH STEWART KOLASCH & BIRCH, PO BOX 747, FALLS CHURCH, VA, 22040-0747, US

(73) Assignee: Tanabe Seiyaku Co., Ltd.

(21) Appl. No.: 11/878,761

(22) Filed: Jul. 26, 2007

Related U.S. Application Data
(60) Provisional application No. 60/820,759, filed on Jul. 28, 2006.

(51) Int. Cl. A61K 31/7056; A61K 31/381; A61P 3/00; C07D 409/10; C07H 19/044

(52) U.S. Cl. 514/43; 514/443; 514/444; 536/28.6; 549/28; 549/58; 549/60

(57) ABSTRACT

Novel compounds of formula (A) or a pharmaceutically acceptable salt thereof:

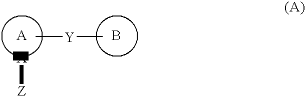

wherein symbols are as defined in claims, which are useful as SGLT inhibitors and for treatment of diabetes and related diseases.

### TECHNICAL FIELD

The present invention relates to novel compounds possessing activity as inhibitors of sodium-dependent glucose transporters (SGLT) found in the intestine or kidney.

### BACKGROUND ART

Diet therapy and exercise therapy are essential in the treatment of diabetes mellitus. When these therapies do not sufficiently control conditions of patients, insulin or anti-diabetic agents are used. At the present, biguanides, sulfonylureas, insulin-sensitizing agents and α-glucosidase inhibitors are used for anti-diabetic agents. However, these anti-diabetic agents have various side effects. For example, biguanides cause lactic acidosis, sulfonylureas cause significant hypoglycemia, insulin-sensitizing agents cause edema and heart failure, and α-glucosidase inhibitors cause abdominal bloating and diarrhea. Under these circumstances, new anti-diabetic drugs that eliminate these side effects are anticipated.

Recently, it has been reported that hyperglycemia participates in the onset and progression of diabetes mellitus. This theory is called glucose toxicity theory. Namely, chronic hyperglycemia leads to decrease of insulin secretion and insulin sensitivity, the plasma glucose level is elevated, and as a result, diabetes mellitus is self-exacerbated [cf., Diabetologia, vol. 28, p. 119 (1985); Diabetes Care, vol. 13, p. 610 (1990), etc.]. Based on this theory, it is expected that normalization of plasma glucose level interrupts the aforementioned self-exacerbating cycle and the prevention or treatment of diabetes mellitus can be achieved.

It is considered that one method for the treatment of hyperglycemia is to excrete an excess amount of glucose directly into urine so that the blood glucose concentration can be normalized. For example, by inhibiting sodium-dependent glucose transporters being present at the proximal convoluted tubule of kidney, the re-absorption of glucose at the kidney is inhibited whereby the excretion of glucose into urine can be promoted and the blood glucose level can be decreased. In fact, it is confirmed that by continuous subcutaneous administration of an SGLT inhibitor, phlorizin, to diabetic animal models, the blood glucose level thereof can be normalized, and that by keeping the blood glucose level normal for a long time, the insulin secretion and insulin resistance can be improved [cf., Journal of Clinical Investigation, vol. 79, p. 1510 (1987); ibid., vol. 80, p. 1037 (1987); ibid., vol. 87, p. 561 (1991), etc.]

In addition, by treating diabetic animal models with an SGLT inhibitor for a long time, insulin secretion response and insulin sensitivity of the animal models are improved without incurring any adverse affects on the kidney or imbalance in blood levels of electrolytes, and as a result, the onset and progress of diabetic nephropathy and diabetic neuropathy are prevented [cf., Journal of Medicinal Chemistry, vol. 42, p. 5311 (1999); British Journal of Pharmacology, vol. 132, p. 578 (2001), etc.].

In view of the above, SGLT inhibitors are expected to improve insulin secretion and insulin resistance by decreasing the blood glucose level in diabetic patients and to prevent the onset and progress of diabetes mellitus and diabetic complications.

WO 2004/014931 discloses aryl O- or N-thioglucopyranosides of the following formula:

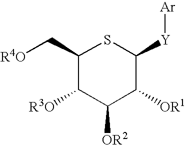

in which Y is O or N.

US 2004/0259819 A1 discloses heterocyclic fluoroglycoside derivatives of the following formula:

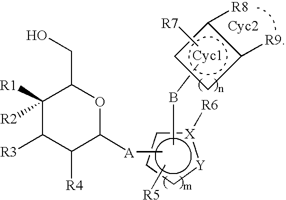

US 2005/0014704 A1 discloses aryl fluoroglycoside derivatives of the following formula:

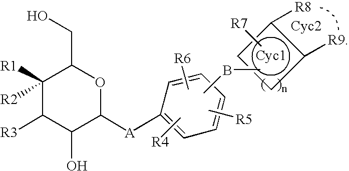

These compounds are described as SGLT inhibitors for treatment of diabetes and related disease.

In addition, WO 98/31697 discloses some aryl C-galactopyranosides such as:

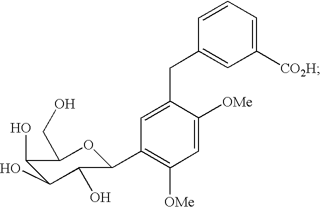

which may be useful for treatment or prevention of inflammatory diseases, autoimmune diseases, and the like.

### DISCLOSURE OF INVENTION

The present invention relates to novel compounds of formula (A), or a pharmaceutically acceptable salt thereof:

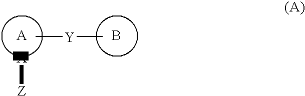

wherein Ring A and Ring B are independently an optionally substituted unsaturated heteromonocyclic ring, an optionally substituted unsaturated fused heterobicyclic ring, or an optionally substituted benzene ring;

X is carbon or nitrogen;

Y is —(CH₂)ₙ— (wherein n is 1 or 2); and

Z is one of the following groups:

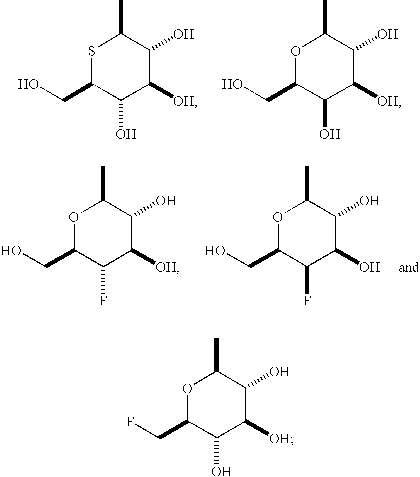

provided that:
(i) when Ring A is an optionally substituted unsaturated fused heterobicyclic ring, Y connects to the X-containing ring of said unsaturated fused heterobicyclic ring,
(ii) when Ring A is an optionally substituted unsaturated fused heterobicyclic ring, Z is:

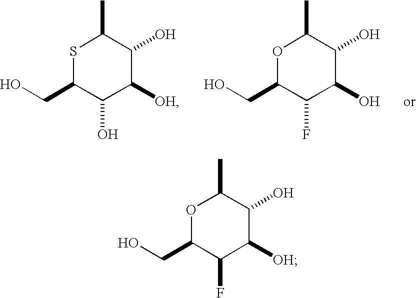

(iii) when both Ring A and Ring B are optionally substituted benzene, Z is:

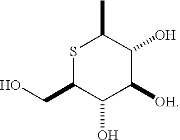

The compounds of formula (A) possess activity as inhibitors of SGLT found in the intestine and kidney of mammals, and are useful in the treatment or prevention of diabetes mellitus and diabetic complications such as diabetic retinopathy, diabetic neuropathy, diabetic nephropathy, and delayed wound healing, and related diseases.

The term “halogen” or “halo” refers to chlorine, bromine, fluorine and iodine, and chlorine and fluorine are preferable.

The term “alkyl” refers to a straight or branched saturated monovalent hydrocarbon chain having 1 to 12 carbon atoms, preferably 1 to 6 carbon atoms, more preferably 1 to 4 carbon atoms, which may optionally be substituted with 1 to 4 substituents described below. Examples of alkyl include methyl, ethyl, propyl, isopropyl, butyl, t-butyl, isobutyl, and various branched chain isomers thereof.

The term “alkylene” refers to a straight or branched divalent hydrocarbon chain containing 1 to 12 carbon atoms, preferably 1 to 6 carbon atoms, more preferably 1 to 4 carbon atoms, which may optionally be substituted with 1 to 4 substituents described below, or which may link at two different carbon atoms of a benzene ring, an unsaturated heteromonocyclic ring, or an unsaturated heterobicyclic ring, thereby forming an annelated five, six or seven membered carbocycle. The annelated five, six or seven membered carbocycle may optionally be substituted with one or more substituents described below, if necessary. Examples of alkylene include methylene, ethylene, propylene, trimethylene, etc.

The term “alkenyl” refers to a straight or branched monovalent hydrocarbon chain containing 2 to 12 carbon atoms, preferably 2 to 6 carbon atoms, more preferably 2 to 4 carbon atoms and containing at least one double bond, which may optionally be substituted with 1 to 4 substituents described below. Examples of alkenyl include vinyl, 2-propenyl, 3-butenyl, 2-butenyl, 4-pentenyl, 3-pentenyl, 2-hexenyl, 3-hexenyl, 2-heptenyl, 3-heptenyl, 4-heptenyl, 3-octenyl, 3-nonenyl, 4-decenyl, 3-undecenyl, 4-dodecenyl, 4,8,12-tetradecatrienyl, etc.

The term “alkenylene” refers to a straight or branched divalent hydrocarbon chain containing 2 to 12 carbon atoms, preferably 2 to 6 carbon atoms, more preferably 2 to 4 carbon atoms and containing at least one double bond, which may optionally be substituted with 1 to 4 substituents described below, or which may link at two different carbon atoms of a benzene ring, an unsaturated hetero monocyclic ring or an unsaturated heterobicyclic ring, thereby forming an annelated five, six or seven membered carbocycle. The annelated five, six or seven membered carbocycle may optionally be substituted with one or more substituents described below. Examples of alkenylene include vinylene, propenylene, butadienylene, etc.

The term “alkynyl” refers to a straight or branched monovalent hydrocarbon chain containing 2 to 12 carbon atoms, preferably 2 to 6 carbon atoms, more preferably 2 to 4 carbon atoms and containing at least one triple bond, which may optionally be substituted with 1 to 4 substituents described below. Examples of alkynyl include 2-propynyl, 3-butynyl, 2-butynyl, 4-pentynyl, 3-pentynyl, 2-hexynyl, 3-hexynyl, 2-heptynyl, 3-heptynyl, 4-heptynyl, 3-octynyl, 3-nonynyl, 4-decynyl, 3-undecynyl, 4-dodecynyl, etc.

The term “cycloalkyl” refers to a monocyclic or bicyclic monovalent saturated hydrocarbon ring containing 3 to 12 carbon atoms, preferably 3 to 7 carbon atoms, which may optionally be substituted with 1 to 4 substituents described below. Examples of cycloalkyl include cyclopropyl, cyclobutyl, cyclopentyl, cyclohexyl, cycloheptyl, cyclooctyl, cyclodecyl, etc.

The term “cycloalkenyl” refers to a monocyclic or bicyclic monovalent unsaturated hydrocarbon ring containing at least one double bond and 4 to 12 carbon atoms, preferably 4 to 7 carbon atoms, which may optionally be substituted with 1 to 4 substituents described below. Examples of cycloalkenyl include cyclopentenyl, cyclopentadienyl, cyclohexenyl, etc.

The term “cycloalkynyl” refers to a monocyclic or bicyclic unsaturated hydrocarbon ring containing at least one triple bond and 6 to 12 carbon atoms, preferably 6 to 8 carbon atoms, which may optionally be substituted with 1 to 4 substituents described below. Examples of cycloalkynyl include cyclooctynyl, cyclodecynyl.

The term “aryl” refers to a monocyclic or bicyclic monovalent aromatic carbocycle containing 6 to 10 carbon atoms, which may optionally be substituted with 1 to 4 substituents described below. Examples of aryl include phenyl, and naphthyl (e.g., 1-naphthyl, 2-naphthyl, etc.).

The term “unsaturated heteromonocyclic ring” refers to an unsaturated 3- to 12-membered, preferably 4- to 7-membered hydrocarbon ring containing at least one double bond or triple bond and at least one heteroatom independently selected from nitrogen, oxygen and sulfur, which may optionally be substituted with 1-4 substituents described below. Examples of unsaturated heteromonocyclic ring include pyridine, pyrimidine, pyrazine, furan, thiophene, pyrrole, imidazole, pyrazole, oxazole, isoxazole, 4,5-dihydroxazolyl, thiazole, isothiazole, thiadiazole, triazole, tetrazole, etc.

The term “unsaturated fused heterobicyclic ring” refers to an unsaturated 7- to 12-membered, preferably 8-to 10-membered fused hydrocarbon bicyclic ring containing at least one double bond or triple bond and at least one heteroatom independently selected from nitrogen, oxygen and sulfur, which may optionally be substituted with 1-4 substituents described below. Examples of unsaturated fused heterobicyclic ring include benzothiophene, indole, tetrahydrobenzothiophene, benzofuran, isoquinoline, thienothiophene, thienopyridine, quinoline, indoline, isoindoline, benzothiazole, benzoxazole, indazole, dihydroisoquinoline, etc.

The term “heterocyclyl” refers to a monovalent group of the above-mentioned unsaturated heteromonocyclic ring or unsaturated fused heterobicyclic ring and a monovalent group of the saturated version of the above-mentioned unsaturated heteromonocyclic or unsaturated fused heterobicyclic ring. If necessary, the heterocyclyl may optionally and independently be substituted with 1 to 4 substituents described below.

The term “alkanoyl” refers to above alkyl linked to carbonyl.

The term “alkoxy” refers to above alkyl linked to oxygen.

The term “alkylthio” refers to above alkyl linked to sulfur.

Substituents for the above groups include, for example, halogen (e.g., fluorine, chlorine, bromine, iodine), nitro, cyano, oxo, hydroxy, mercapto, carboxyl, sulfo, alkyl, alkenyl, alkynyl, cycloalkyl, cycloalkenyl, cycloalkynyl, aryl, heterocyclyl, alkoxy, alkenyloxy, alkynyloxy, cycloalkyloxy, cycloalkenyloxy, cycloalkynyloxy, aryloxy, heterocyclyloxy, alkanoyl, alkenylcarbonyl, alkynylcarbonyl, cycloalkylcarbonyl, cycloalkenylcarbonyl, cycloalkynylcarbonyl, arylcarbonyl, heterocyclylcarbonyl, alkoxycarbonyl, alkenyloxycarbonyl, alkynyloxycarbonyl, cycloalkyloxycarbonyl, cycloalkenyloxycarbonyl, cycloalkynyloxycarbonyl, aryloxycarbonyl, heterocyclyloxycarbonyl, alkanoyloxy, alkenylcarbonyloxy, alkynylcarbonyloxy, cycloalkylcarbonyloxy, cycloalkenylcarbonyloxy, cycloalkynylcarbonyloxy, arylcarbonyloxy, heterocyclylcarbonyloxy, alkylthio, alkenylthio, alkynylthio, cycloalkylthio, cycloalkenylthio, cycloalkynylthio, arylthio, heterocyclylthio, amino, mono- or di-alkylamino, mono- or di-alkanoylamino, mono- or di-alkoxycarbonylamino, mono- or di-arylcarbonylamino, alkylsulfinylamino, alkylsulfonylamino, arylsulfinylamino, arylsulfonylamino, carbamoyl, mono- or di-alkylcarbamoyl, mono- or di-arylcarbamoyl, alkylsulfinyl, alkenylsulfinyl, alkynylsulfinyl, cycloalkylsulfinyl, cycloalkenylsulfinyl, cycloalkynylsulfinyl, arylsulfinyl, heterocyclylsulfinyl, alkylsulfonyl, alkenylsulfonyl, alkynylsulfonyl, cycloalkylsulfonyl, cycloalkenylsulfonyl, cycloalkynylsulfonyl, arylsulfonyl, and heterocyclylsulfonyl. Each of these groups may optionally have a substituent selected from these substituents.

Further, the terms such as “haloalkyl”, “halo-lower alkyl”, “haloalkoxy”, “halo-lower alkoxy”, “halophenyl”, and “haloheterocyclyl” refer to alkyl, lower alkyl, alkoxy, lower alkoxy, phenyl and heterocyclyl each of which is substituted with one or more halogen atoms, respectively. These terms preferably include alkyl, lower alkyl, alkoxy, lower alkoxy, phenyl and heterocyclyl each of which is substituted with 1 to 7 halogen atoms, more preferably 1 to 5 halogen atoms. Further, the terms “halopyridyl” and “halothienyl” respectively refer to pyridyl and thienyl being substituted with one or more halogen atoms, preferably Cl or F. Examples of “haloalkyl”, “haloalkoxy”, “halophenyl”, “halopyridyl” and “halothienyl” include CHF₂, CF₃, CHF₂O, CF₃O, CF₃CH₂, CF₃CH₂O, FCH₂CH₂O, ClCH₂CH₂O, FC₆H₄, ClC₆H₄, BrC₆H₄, IC₆H₄, FC₅H₃N, ClC₅H₃N, BrC₅H₃N, FC₄H₂S, ClC₄H₂S, and BrC₄H₂S.

Similarly, the terms such as “hydroxyalkyl”, “hydroxy-lower alkyl”, “hydroxyalkoxy”, “hydroxy-lower alkoxy”, and “hydroxyphenyl” include alkyl, lower alkyl, alkoxy, lower alkoxy, and phenyl each of which is substituted with one or more hydroxy. These terms preferably refer to alkyl, lower alkyl, alkoxy, lower alkoxy, and phenyl each of which is substituted with 1 to 4 hydroxy, more preferably with 1 to 2 hydroxy. Further, the terms such as “alkoxyalkyl”, “lower alkoxyalkyl”, “alkoxy-lower alkyl”, “lower alkoxy-lower alkyl”, “alkoxyalkoxy”, “lower alkoxyalkoxy”, “alkoxy-lower alkoxy”, “lower alkoxy-lower alkoxy”, “alkoxyphenyl”, and “lower alkoxyphenyl” refer to alkyl, lower alkyl, alkoxy, lower alkoxy, and phenyl each of which is substituted with one or more alkoxy. These terms preferably refer to alkyl, lower alkyl, alkoxy, lower alkoxy, and phenyl each of which is substituted with 1 to 4 alkoxy, more preferably by 1 to 2 alkoxy.

Similarly, the term “cyanophenyl” refers to phenyl being substituted with one or more cyano.

The terms “arylakyl” and “arylalkoxy” as used alone or as a part of another group refer to alkyl and alkoxy each substituted with aryl, respectively.

The term “lower” used in the specification and claims refers to a straight or branched carbon chain having 1 to 6 carbon atoms, unless defined otherwise. Preferably, it means a straight or branched carbon chain having 1 to 4 carbon atoms.

The term “prodrug” refers to an ester or carbonate, which can be formed by condensing one or more hydroxy groups of the compounds of formula (A) with an appropriate acylating agent in a conventional method to produce acetate, pivalate, methylcarbonate, benzoate, etc. The term “prodrug” also refers to an ester or amide, which can be formed by condensing one or more hydroxy groups of the compounds of formula (A) with an amino acid (e.g., α-amino acid, and β-amino acid, etc.) in a conventional method.

The pharmaceutically acceptable salts of the compounds of formula (A) include, for example, a salt with an alkali metal such as lithium, sodium, potassium, etc.; salt with an alkaline earth metal such as calcium, magnesium, etc.; salt with zinc or aluminum; salt with an organic base such as ammonium, choline, diethanolamine, lysine, ethylenediamine, t-butylamine, t-octylamine, tris(hydroxymethyl)aminomethane, N-methyl-glucosamine, triethanolamine and dehydroabietylamine; salt with an inorganic acid such as hydrochloric acid, hydrobromic acid, hydroiodic acid, sulfuric acid, nitric acid, phosphoric acid, etc.; or a salt with an organic acid such as formic acid, acetic acid, propionic acid, oxalic acid, malonic acid, succinic acid, fumaric acid, maleic acid, lactic acid, malic acid, tartaric acid, citric acid, methanesulfonic acid, ethanesulfonic acid, benzenesulfonic acid, etc.; or a salt with an acidic amino acid such as aspartic acid, glutamic acid, etc.

The compounds of the present invention may optionally have one or more asymmetric carbon atoms contained in any substituents, and the compounds of formula (A) may exist in the form of enantiomer or diastereomer, or a mixture thereof. The compounds of the present invention include a mixture of stereoisomers, or each pure or substantially pure isomer. In case that the compounds of formula (A) are obtained in the form of a diastereomer or enantiomer, they can be separated by a conventional method well known in the art such as chromatography or fractional crystallization.

In addition, the compounds of formula (A) include an intramolecular salt, hydrate, solvate or polymorphism thereof.

In an embodiment of the present invention, Ring A is an optionally substituted unsaturated heteromonocyclic ring, and Z is:

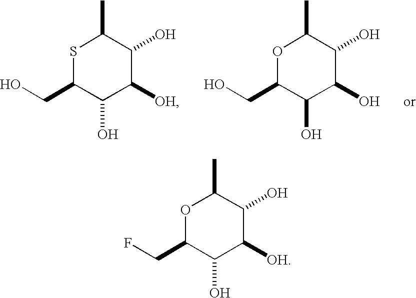

In an alternative embodiment of the present invention, the optionally substituted unsaturated heteromonocyclic ring is preferably an unsaturated heteromonocyclic ring which may optionally be substituted with 1-5 substituents independently selected from the group consisting of: halogen, nitro, cyano, oxo, hydroxyl, mercapto, carboxyl, sulfo, alkyl, alkenyl, alkynyl, cycloalkyl, cycloalkenyl, cycloalkynyl, aryl, heterocyclyl, alkoxy, alkenyloxy, alkynyloxy, cycloalkyloxy, cycloalkenyloxy, cycloalkynyloxy, aryloxy, heterocyclyloxy, alkanoyl, alkenylcarbonyl, alkynylcarbonyl, cycloalkylcarbonyl, cycloalkenylcarbonyl, cycloalkynylcarbonyl, arylcarbonyl, heterocyclylcarbonyl, alkoxycarbonyl, alkenyloxycarbonyl, alkynyloxycarbonyl, cycloalkyloxycarbonyl, cycloalkenyloxycarbonyl, cycloalkynyloxycarbonyl, aryloxycarbonyl, heterocyclyloxycarbonyl, alkanoyloxy, alkenylcarbonyloxy, alkynylcarbonyloxy, cycloalkylcarbonyloxy, cycloalkenylcarbonyloxy, cycloalkynylcarbonyloxy, arylcarbonyloxy, heterocyclylcarbonyloxy, alkylthio, alkenylthio, alkynylthio, cycloalkylthio, cycloalkenylthio, cycloalkynylthio, arylthio, heterocyclylthio, amino, mono- or di-alkylamino, mono- or di-alkanoylamino, mono- or di-alkoxycarbonylamino, mono- or di-arylcarbonylamino, alkylsulfinylamino, alkylsulfonylamino, arylsulfinylamino, arylsulfonylamino, carbamoyl, mono- or di-alkylcarbamoyl, mono- or di-arylcarbamoyl, alkylsulfinyl, alkenylsulfinyl, alkynylsulfinyl, cycloalkylsulfinyl, cycloalkenylsulfinyl, cycloalkynylsulfinyl, arylsulfinyl, heterocyclylsulfinyl, alkylsulfonyl, alkenylsulfonyl, alkynylsulfonyl, cycloalkylsulfonyl, cycloalkenylsulfonyl, cycloalkynylsulfonyl, arylsulfonyl, and heterocyclylsulfonyl, wherein each of these substituents may optionally be further substituted with a group selected from these substituents;

the optionally substituted unsaturated fused heterobicyclic ring is preferably an unsaturated fused heterobicyclic ring which may optionally be substituted with 1-5 substituents independently selected from the group consisting of: halogen, nitro, cyano, oxo, hydroxy, mercapto, carboxyl, sulfo, alkyl, alkenyl, alkynyl, cycloalkyl, cycloalkenyl, cycloalkynyl, aryl, heterocyclyl, alkoxy, alkenyloxy, alkynyloxy, cycloalkyloxy, cycloalkenyloxy, cycloalkynyloxy, aryloxy, heterocyclyloxy, alkanoyl, alkenylcarbonyl, alkynylcarbonyl, cycloalkylcarbonyl, cycloalkenylcarbonyl, cycloalkynylcarbonyl, arylcarbonyl, heterocyclylcarbonyl, alkoxycarbonyl, alkenyloxycarbonyl, alkynyloxycarbonyl, cycloalkyloxycarbonyl, cycloalkenyloxycarbonyl, cycloalkynyloxycarbonyl, aryloxycarbonyl, heterocyclyloxycarbonyl, alkanoyloxy, alkenylcarbonyloxy, alkynylcarbonyloxy, cycloalkylcarbonyloxy, cycloalkenylcarbonyloxy, cycloalkynylcarbonyloxy, arylcarbonyloxy, heterocyclylcarbonyloxy, alkylthio, alkenylthio, alkynylthio, cycloalkylthio, cycloalkenylthio, cycloalkynylthio, arylthio, heterocyclylthio, amino, mono- or di-alkylamino, mono- or di-alkanoyl-amino, mono- or di-alkoxycarbonylamino, mono- or di-arylcarbonylamino, alkylsulfinylamino, alkylsulfonylamino, arylsulfinylamino, arylsulfonylamino, carbamoyl, mono- or di-alkylcarbamoyl, mono- or di-arylcarbamoyl, alkylsulfinyl, alkenylsulfinyl, alkynylsulfinyl, cycloalkylsulfinyl, cycloalkenylsulfinyl, cycloalkynylsulfinyl, arylsulfinyl, heterocyclylsulfinyl, alkylsulfonyl, alkenylsulfonyl, alkynylsulfonyl, cycloalkylsulfonyl, cycloalkenylsulfonyl, cycloalkynylsulfonyl, arylsulfonyl, heterocyclylsulfonyl, wherein each of these substituent may optionally have a substituent selected from these substituents; and

the optionally substituted benzene ring is preferably a benzene ring which may optionally be substituted with 1-5 substituents independently selected from the group consisting of: halogen, nitro, cyano, hydroxy, mercapto, carboxyl, sulfo, alkyl, alkenyl, alkynyl, cycloalkyl, cycloalkenyl, cycloalkynyl, aryl, heterocyclyl, alkoxy, alkenyloxy, alkynyloxy, cycloalkyloxy, cycloalkenyloxy, cycloalkynyloxy, aryloxy, heterocyclyloxy, alkanoyl, alkenylcarbonyl, alkynylcarbonyl, cycloalkylcarbonyl, cycloalkenylcarbonyl, cycloalkynylcarbonyl, arylcarbonyl, heterocyclylcarbonyl, alkoxycarbonyl, alkenyloxycarbonyl, alkynyloxycarbonyl, cycloalkyloxycarbonyl, cycloalkenyloxycarbonyl, cycloalkynyloxycarbonyl, aryloxycarbonyl, heterocyclyloxycarbonyl, alkanoyloxy, alkenylcarbonyloxy, alkynylcarbonyloxy, cycloalkylcarbonyloxy, cycloalkenylcarbonyloxy, cycloalkynylcarbonyloxy, arylcarbonyloxy, heterocyclylcarbonyloxy, alkylthio, alkenylthio, alkynylthio, cycloalkylthio, cycloalkenylthio, cycloalkynylthio, arylthio, heterocyclylthio, amino, mono- or di-alkylamino, mono- or di-alkanoylamino, mono- or di-alkoxycarbonylamino, mono- or di-arylcarbonylamino, alkylsulfinylamino, alkylsulfonylamino, arylsulfinylamino, arylsulfonylamino, carbamoyl, mono- or di-alkylcarbamoyl, mono- or di-arylcarbamoyl, alkylsulfinyl, alkenylsulfinyl, alkynylsulfinyl, cycloalkylsulfinyl, cycloalkenylsulfinyl, cycloalkynylsulfinyl, arylsulfinyl, heterocyclylsulfinyl, alkylsulfonyl, alkenylsulfonyl, alkynylsulfonyl, cycloalkylsulfonyl, cycloalkenylsulfonyl, cycloalkynylsulfonyl, arylsulfonyl, heterocyclylsulfonyl, alkylene, alkyleneoxy, alkylenedioxy, and alkenylene, wherein each of these substituents may optionally be further substituted with these substituents.

The optionally substituted benzene ring also refers to a benzene ring substituted with alkylene or alkenylene to form an annelated carbocycle together with the carbon atoms to which they are attached. Examples of such annelated carbocycles include fused benzene, fused cyclopentene, and the like.

Preferable examples of the optionally substituted unsaturated heteromonocyclic ring include an unsaturated heteromonocyclic ring which may optionally be substituted with 1-3 substituents independently selected from the group consisting of: halogen, hydroxy, alkoxy, alkyl, haloalkyl, haloalkoxy, hydroxyalkyl, alkoxyalkyl, alkoxyalkoxy, alkenyl, alkynyl, cycloalkyl, cycloalkenyl, cycloalkyloxy, aryl, aryloxy, arylalkoxy, cyano, nitro, amino, mono- or di-alkylamino, alkanoylamino, alkoxycarbonylamino, carboxyl, alkoxycarbonyl, carbamoyl, mono- or di-alkylcarbamoyl, alkanoyl, alkylsulfonylamino, arylsulfonylamino, alkylsulfinyl, alkylsulfonyl, arylsulfonyl, heterocyclyl, and oxo.

Preferable examples of the optionally substituted unsaturated fused heterobicyclic ring include an unsaturated fused heterobicyclic ring which may optionally be substituted with 1-3 substituents independently selected from the group consisting of: halogen, hydroxy, alkoxy, alkyl, haloalkyl, haloalkoxy, hydroxyalkyl, alkoxyalkyl, alkoxyalkoxy, alkenyl, alkynyl, cycloalkyl, cycloalkenyl, cycloalkyloxy, aryl, aryloxy, arylalkoxy, cyano, nitro, amino, mono- or di-alkylamino, alkanoylamino, alkoxycarbonylamino, carboxyl, alkoxycarbonyl, carbamoyl, mono- or di-alkylcarbamoyl, alkanoyl, alkylsulfonylamino, arylsulfonylamino, alkylsulfinyl, alkylsulfonyl, arylsulfonyl, heterocyclyl, and oxo.

Preferable examples of the optionally substituted benzene ring include a benzene ring which may optionally be substituted with 1-3 substituents independently selected from the group consisting of: halogen, hydroxy, alkoxy, alkyl, haloalkyl, haloalkoxy, hydroxyalkyl, alkoxyalkyl, alkoxyalkoxy, alkenyl, alkynyl, cycloalkyl, cycloalkenyl, cycloalkyloxy, aryl, aryloxy, arylalkoxy, cyano, nitro, amino, mono- or di-alkylamino, alkanoylamino, alkoxycarbonylamino, carboxyl, alkoxycarbonyl, carbamoyl, mono- or di-alkylcarbamoyl, alkanoyl, alkylsulfonylamino, arylsulfonylamino, alkylsulfinyl, alkylsulfonyl, arylsulfonyl, heterocyclyl, alkylene, alkyleneoxy, alkylenedioxy, and alkenylene.

In an alternative embodiment of the present invention, the optionally substituted unsaturated heteromonocyclic ring is preferably an unsaturated heteromonocyclic ring which may optionally be substituted with 1-3 substituents, independently selected from the group consisting of: halogen, hydroxy, cyano, nitro, alkyl, alkenyl, alkynyl, cycloalkyl, alkoxy, alkanoyl, alkylthio, alkylsulfonyl, alkylsulfinyl, amino, mono- or di-alkylamino, alkanoylamino, alkoxycarbonylamino, sulfamoyl, mono- or di-alkylsulfamoyl, carboxyl, alkoxycarbonyl, carbamoyl, mono- or di-alkylcarbamoyl, alkylsulfonylamino, phenyl, phenoxy, phenylsulfonylamino, phenylsulfonyl, heterocyclyl, and oxo;

the optionally substituted unsaturated fused heterobicyclic ring is preferably an unsaturated fused heterobicyclic ring which may optionally be substituted with 1-3 substituents independently selected from the group consisting of: halogen, hydroxy, cyano, nitro, alkyl, alkenyl, alkynyl, cycloalkyl, alkoxy, alkylthio, alkylsulfonyl, alkylsulfinyl, amino, mono- or di-alkylamino, alkanoylamino, alkoxycarbonylamino, sulfamoyl, mono- or di-alkyl-sulfamoyl, carboxyl, alkoxycarbonyl, carbamoyl, mono- or di-alkylcarbamoyl, alkanoyl, alkylsulfonylamino, phenyl, phenoxy, phenylsulfonylamino, phenylsulfonyl, heterocyclyl, and oxo; and

the optionally substituted benzene ring is preferably a benzene ring which may optionally be substituted with 1-3 substituents, independently selected from the group consisting of: halogen, hydroxy, cyano, nitro, alkyl, alkenyl, alkynyl, cycloalkyl, alkoxy, alkanoyl, alkylthio, alkylsulfonyl, alkylsulfinyl, amino, mono- or di-alkylamino, alkanoylamino, alkoxycarbonylamino, sulfamoyl, mono- or di-alkylsulfamoyl, carboxyl, alkoxycarbonyl, carbamoyl, mono- or di-alkylcarbamoyl, alkylsulfonylamino, phenyl, phenoxy, phenylsulfonylamino, phenylsulfonyl, heterocyclyl, alkylene, and alkenylene;

wherein each of the above-mentioned substituents on the unsaturated heteromonocyclic ring, the unsaturated fused heterobicyclic ring and the benzene ring may further be substituted with 1-3 substituents, independently selected from the group consisting of: halogen, hydroxy, cyano, alkyl, haloalkyl, alkoxy, haloalkoxy, alkanoyl, alkylthio, alkylsulfonyl, mono- or di-alkylamino, carboxyl, alkoxycarbonyl, phenyl, alkyleneoxy, alkylenedioxy, oxo, carbamoyl, mono- or di-alkylcarbamoyl.

In an alternative embodiment of the invention, the optionally substituted unsaturated heteromonocyclic ring is an unsaturated heteromonocyclic ring which may optionally be substituted with 1-3 substituents, independently selected from the group consisting of: halogen, cyano, alkyl, alkoxy, alkanoyl, mono- or di-alkylamino, alkanoylamino, alkoxycarbonylamino, carboxyl, alkoxycarbonyl, carbamoyl, mono- or di-alkylcarbamoyl, phenyl, heterocyclyl, and oxo;

the optionally substituted unsaturated fused heterobicyclic ring is an unsaturated fused heterobicyclic ring which may optionally be substituted with 1-3 substituents independently selected from the group consisting of: halogen, cyano, alkyl, alkoxy, alkanoyl, mono- or di-alkylamino, alkanoylamino, alkoxycarbonylamino, carboxy, alkoxycarbonyl, carbamoyl, mono- or di-alkylcarbamoyl, phenyl, heterocyclyl, and oxo; and

the optionally substituted benzene ring is a benzene ring which may optionally be substituted with 1-3 substituents, independently selected from the group consisting of: halogen, cyano, alkyl, alkoxy, alkanoyl, mono- or di-alkylamino, alkanoylamino, alkoxycarbonylamino, carboxyl, alkoxycarbonyl, carbamoyl, mono- or di-alkylcarbamoyl, phenyl, heterocyclyl, alkylene, and alkenylene;

wherein each of the above-mentioned substituents on the unsaturated heteromonocyclic ring, the unsaturated fused heterobicyclic ring and the benzene ring may further be substituted with 1-3 substituents, independently selected from the group consisting of: halogen, cyano, alkyl, haloalkyl, alkoxy, haloalkoxy, alkanoyl, mono- or di-alkylamino, carboxyl, hydroxy, phenyl, alkylenedioxy, alkyleneoxy, alkoxycarbonyl, carbamoyl and mono- or di-alkylcarbamoyl.

In a preferable embodiment of the present invention,

(1) Ring A is an unsaturated heteromonocyclic ring which may optionally be substituted with 1-3 substituents independently selected from the group consisting of: halogen, hydroxy, cyano, nitro, alkyl, alkenyl, alkynyl, cycloalkyl, alkoxy, alkanoyl, alkylthio, alkylsulfonyl, alkylsulfinyl, amino, mono- or di-alkylamino, sulfamoyl, mono- or di-alkylsulfamoyl, carboxyl, alkoxycarbonyl, carbamoyl, mono- or di-alkylcarbamoyl, alkylsulfonylamino, phenyl, phenoxy, phenylsulfonylamino, phenylsulfonyl, heterocyclyl, and oxo, and

Ring B is an unsaturated heteromonocyclic ring, an unsaturated fused heterobicyclic ring, or a benzene ring; each of which may optionally be substituted with 1-3 substituents, independently selected from the group consisting of: halogen, hydroxy, cyano, nitro, alkyl, alkenyl, alkynyl, cycloalkyl, alkoxy, alkanoyl, alkylthio, alkylsulfonyl, alkylsulfinyl, amino, mono- or di-alkylamino, sulfamoyl, mono- or di-alkylsulfamoyl, carboxyl, alkoxycarbonyl, carbamoyl, mono- or di-alkylcarbamoyl, alkylsulfonylamino, phenyl, phenoxy, phenylsulfonylamino, phenylsulfonyl, heterocyclyl, alkylene, and alkenylene;

wherein each of the above-mentioned substituents on Ring A and Ring B may optionally be substituted with 1-3 substituents, independently selected from the group consisting of: halogen, cyano, alkyl, haloalkyl, alkoxy, haloalkoxy, alkanoyl, mono- or di-alkylamino, carboxyl, hydroxy, phenyl, alkylenedioxy, alkyleneoxy, alkoxycarbonyl, carbamoyl and mono- or di-alkylcarbamoyl;

(2) Ring A is a benzene ring which may optionally be substituted with 1-3 substituents, independently selected from the group consisting of: halogen, hydroxy, cyano, nitro, alkyl, alkenyl, alkynyl, cycloalkyl, alkoxy, alkanoyl, alkylthio, alkylsulfonyl, alkylsulfinyl, amino, mono- or di-alkylamino, alkanoylamino, sulfamoyl, mono- or di-alkylsulfamoyl, carboxyl, alkoxycarbonyl, carbamoyl, mono- or di-alkylcarbamoyl, alkylsulfonylamino, phenyl, phenoxy, phenylsulfonylamino, phenylsulfonyl, heterocyclyl, alkylene, and alkenylene, and

Ring B is an unsaturated heteromonocyclic ring or an unsaturated fused heterobicyclic ring, each of which may optionally be substituted with 1-3 substituents, independently selected from the group consisting of: halogen, hydroxy, cyano, nitro, alkyl, alkenyl, alkynyl, cycloalkyl, alkoxy, alkanoyl, alkylthio, alkylsulfonyl, alkylsulfinyl, amino, mono- or di-alkylamino, sulfamoyl, mono- or di-alkylsulfamoyl, carboxyl, alkoxycarbonyl, carbamoyl, mono- or di-alkylcarbamoyl, alkylsulfonylamino, phenyl, phenoxy, phenylsulfonylamino, phenylsulfonyl, heterocyclyl, alkylene and oxo;

wherein each of the above-mentioned substituents on Ring A and Ring B may optionally be substituted with 1-3 substituents, independently selected from the group consisting of: halogen, cyano, alkyl, haloalkyl, alkoxy, haloalkoxy, alkanoyl, mono- or di-alkylamino, carboxyl, hydroxy, phenyl, alkylenedioxy, alkyleneoxy, alkoxycarbonyl, carbamoyl and mono- or di-alkylcarbamoyl; or

(3) Ring A is an unsaturated fused heterobicyclic ring which may optionally be substituted with 1-3 substituents, independently selected from the group consisting of: halogen, hydroxy, cyano, nitro, alkyl, alkenyl, alkynyl, cycloalkyl, alkoxy, alkanoyl, alkylthio, alkylsulfonyl, alkylsulfinyl, amino, mono- or di-alkylamino, sulfamoyl, mono- or di-alkylsulfamoyl, carboxyl, alkoxycarbonyl, carbamoyl, mono- or di-alkylcarbamoyl, alkylsulfonylamino, phenyl, phenoxy, phenylsulfonylamino, phenylsulfonyl, heterocyclyl, and oxo, and

Ring B is an unsaturated heteromonocyclic ring, an unsaturated fused heterobicyclic ring, or a benzene ring, each of which may optionally be substituted with 1-3 substituents, independently selected from the group consisting of: halogen, hydroxy, cyano, nitro, alkyl, alkenyl, alkynyl, cycloalkyl, alkoxy, alkanoyl, alkylthio, alkylsulfonyl, alkylsulfinyl, amino, mono- or di-alkylamino, sulfamoyl, mono- or di-alkylsulfamoyl, carboxyl, alkoxycarbonyl, carbamoyl, mono- or di-alkylcarbamoyl, alkylsulfonylamino, phenyl, phenoxy, phenylsulfonylamino, phenylsulfonyl, heterocyclyl, alkylene and oxo;

wherein each of the above-mentioned substituents on Ring A and Ring B may optionally be substituted with 1-3 substituents, independently selected from the group consisting of: halogen, cyano, alkyl, haloalkyl, alkoxy, haloalkoxy, alkanoyl, mono- or di-alkylamino, carboxyl, hydroxy, phenyl, alkylenedioxy, alkyleneoxy, alkoxycarbonyl, carbamoyl and mono- or di-alkylcarbamoyl.

In another preferable embodiment of the present invention,

(1) Ring A is an unsaturated heteromonocyclic ring which may optionally be substituted with halogen, lower alkyl, halo-lower alkyl, lower alkoxy, or oxo, and Ring B is (a) a benzene ring which may optionally be substituted with a group selected from: halogen; cyano; lower alkyl; halo-lower alkyl; lower alkoxy; halo-lower alkoxy; mono- or di-lower alkylamino; phenyl optionally substituted with halogen, cyano, lower alkyl, halo-lower alkyl, lower alkoxy, or mono- or di-lower alkylamino; and heterocyclyl optionally substituted with halogen, cyano, lower alkyl, halo-lower alkyl, lower alkoxy, or mono- or di-lower alkylamino; (b) an unsaturated heteromonocyclic ring which may optionally be substituted with a group selected from: halogen; cyano; lower alkyl; halo-lower alkyl; lower alkoxy; halo-lower alkoxy; mono- or di-lower alkylamino; phenyl optionally substituted with halogen, cyano, lower alkyl, halo-lower alkyl, lower alkoxy, or mono- or di-lower alkylamino; and heterocyclyl optionally substituted with halogen, cyano, lower alkyl, halo-lower alkyl, lower alkoxy, or mono- or di-lower alkylamino; or (c) an unsaturated fused heterobicyclic ring which may optionally be substituted with a group selected from: halogen; cyano; lower alkyl; halo-lower alkyl; lower alkoxy; halo-lower alkoxy; mono- or di-lower alkylamino; phenyl optionally substituted with halogen, cyano, lower alkyl, halo-lower alkyl, lower alkoxy, or mono- or di-lower alkylamino; and heterocyclyl optionally substituted with halogen, cyano, lower alkyl, halo-lower alkyl, lower alkoxy, or mono- or di-lower alkylamino;
(2) Ring A is a benzene ring which may optionally be substituted with halogen, lower alkyl, halo-lower alkyl, lower alkoxy, phenyl, or lower alkenylene, and Ring B is (a) an unsaturated heteromonocyclic ring which may optionally be substituted with a group selected from: halogen; cyano; lower alkyl; halo-lower alkyl; phenyl-lower alkyl; lower alkoxy; halo-lower alkoxy; mono- or di-lower alkylamino; phenyl optionally substituted with halogen, cyano, lower alkyl, halo-lower alkyl, lower alkoxy, halo-lower alkoxy, mono- or di-lower alkylamino, carbamoyl, or mono- or di-lower alkylcarbamoyl; and heterocyclyl optionally substituted with halogen, cyano, lower alkyl, halo-lower alkyl, lower alkoxy, halo-lower alkoxy, mono- or di-lower alkylamino, carbamoyl or mono- or di-lower alkylcarbamoyl; (b) an unsaturated fused heterobicyclic ring which may optionally be substituted with a group selected from: halogen; cyano; lower alkyl; halo-lower alkyl; phenyl-lower alkyl; lower alkoxy; halo-lower alkoxy; mo- or di-lower alkylamino; phenyl optionally substituted with halogen, cyano, lower alkyl, halo-lower alkyl, lower alkoxy, or mono- or di-lower alkylamino; and heterocyclyl optionally substituted with halogen, cyano, lower alkyl, halo-lower alkyl, lower alkoxy, or mono- or di-lower alkylamino; or
(3) Ring A is an unsaturated fused heterobicyclic ring which may optionally be substituted with halogen, lower alkyl, halo-lower alkyl, lower alkoxy, or oxo, and Ring B is (a) a benzene ring which may optionally be substituted with a group selected from: halogen; cyano; lower alkyl; halo-lower alkyl; lower alkoxy; halo-lower alkoxy; mo- or di-lower alkylamino; phenyl optionally substituted with halogen, cyano, lower alkyl, halo-lower alkyl, lower alkoxy, or mono- or di-lower alkylamino; and heterocyclyl optionally substituted with halogen, cyano, lower alkyl, halo-lower alkyl, lower alkoxy, or mono- or di-lower alkylamino; (b) an unsaturated heteromonocyclic ring which may optionally be substituted with a group selected from: halogen; cyano; lower alkyl; halo-lower alkyl; lower alkoxy; halo-lower alkoxy; mono- or di-lower alkylamino; phenyl optionally substituted with halogen, cyano, lower alkyl, halo-lower alkyl, lower alkoxy, or mono- or di-lower alkylamino; and heterocyclyl optionally substituted with halogen, cyano, lower alkyl, halo-lower alkyl, lower alkoxy, or mono- or di-lower alkylamino; or (c) an unsaturated fused heterobicyclic ring which may optionally be substituted with a group selected from: halogen; cyano; lower alkyl; halo-lower alkyl; lower alkoxy; halo-lower alkoxy; mo- or di-lower alkylamino; phenyl optionally substituted with halogen, cyano, lower alkyl, halo-lower alkyl, lower alkoxy, or mono- or di-lower alkylamino; and heterocyclyl optionally substituted with halogen, cyano, lower alkyl, halo-lower alkyl, lower alkoxy, or mono- or di-lower alkylamino.

In another preferable embodiment of the present invention, Y is —CH₂— and is linked at the 3-position of Ring A, with respect to X being the 1-position.

In still another preferable embodiment of the present invention, (1) Ring A is a benzene ring which may optionally be substituted with 1-3 substituents, independently selected from the group consisting of: halogen, lower alkyl optionally substituted with halogen or lower alkoxy, lower alkoxy optionally substituted with halogen or lower alkoxy, cycloalkyl, cycloalkoxy, phenyl, and lower alkenylene, and

Ring B is an unsaturated heteromonocyclic ring or an unsaturated fused heterobicyclic ring, each of which may optionally be substituted with 1-3 substituents, independently selected from the group consisting of: halogen; lower alkyl optionally substituted with halogen, lower alkoxy or phenyl; lower alkoxy optionally substituted with halogen or lower alkoxy; cycloalkyl; cycloalkoxy; phenyl optionally substituted with halogen, cyano, lower alkyl, halo-lower alkyl, lower alkoxy, halo-lower alkoxy, mono- or di-lower alkylamino, carbamoyl or mono- or di-lower alkylcarbamoyl; heterocyclyl optionally substituted with halogen, cyano, lower alkyl, halo-lower alkyl, lower alkoxy, halo-lower alkoxy, mono- or di-lower alkylamino, carbamoyl or mono- or di-lower alkylcarbamoyl; and oxo,
(2) Ring A is an unsaturated heteromonocyclic ring which may optionally be substituted with 1-3 substituents, independently selected from the group consisting of: halogen, lower alkyl optionally substituted with lower alkoxy, lower alkoxy optionally substituted with halogen or lower alkoxy, cycloalkyl, cycloalkoxy, and oxo, and Ring B is a benzene ring which may optionally be substituted with 1-3 substituents, independently selected from the group consisting of: halogen; lower alkyl optionally substituted with halogen, lower alkoxy or phenyl; lower alkoxy optionally substituted with halogen or lower alkoxy; cycloalkyl; cycloalkoxy; phenyl optionally substituted with halogen, cyano, lower alkyl, halo-lower alkyl, lower alkoxy or halo-lower alkoxy; heterocyclyl optionally substituted with halogen, cyano, lower alkyl, halo-lower alkyl, lower alkoxy or halo-lower alkoxy; and lower alkylene,
(3) Ring A is an unsaturated heteromonocyclic ring which may optionally be substituted with 1-3 substituents, independently selected from the group consisting of: halogen, lower alkyl optionally substituted with halogen or lower alkoxy, lower alkoxy optionally substituted with halogen or lower alkoxy, cycloalkyl, cycloalkoxy, and oxo, Ring B is an unsaturated heteromonocyclic ring or an unsaturated fused heterobicyclic ring, each of which may optionally be substituted with 1-3 substituents, independently selected from the group consisting of: halogen; lower alkyl optionally substituted with halogen, lower alkoxy or phenyl; lower alkoxy optionally substituted with halogen or lower alkoxy; cycloalkyl; cycloalkoxy; phenyl optionally substituted with halogen, cyano, lower alkyl, halo-lower alkyl, lower alkoxy or halo-lower alkoxy; heterocyclyl optionally substituted with halogen, cyano, lower alkyl, halo-lower alkyl, lower alkoxy or halo-lower alkoxy; and oxo;
(4) Ring A is an unsaturated fused heterobicyclic ring which may optionally be substituted with 1-3 substituents, independently selected from the group consisting of: halogen, lower alkyl optionally substituted with halogen or lower alkoxy, lower alkoxy optionally substituted with halogen or lower alkoxy, cycloalkyl, cycloalkoxy, and oxo, Ring B is a benzene ring or an unsaturated heteromonocyclic ring, each of which may optionally be substituted with 1-3 substituents, independently selected from the group consisting of: halogen; lower alkyl optionally substituted with halogen, lower alkoxy or phenyl; lower alkoxy optionally substituted with halogen or lower alkoxy; cycloalkyl; cycloalkoxy; phenyl optionally substituted with halogen, cyano, lower alkyl, halo-lower alkyl, lower alkoxy or halo-lower alkoxy; heterocyclyl optionally substituted with halogen, cyano, lower alkyl, halo-lower alkyl, lower alkoxy or halo-lower alkoxy; and lower alkylene, or
(5) Ring A is an unsaturated heteromonocyclic ring which may optionally be substituted with 1-3 substituents, independently selected from the group consisting of: halogen, lower alkyl optionally substituted with lower alkoxy, lower alkoxy optionally substituted with halogen or lower alkoxy, cycloalkyl, cycloalkoxy, and oxo, Ring B is an unsaturated heteromonocyclic ring or an unsaturated fused heterobicyclic ring, each of which may optionally be substituted with 1-3 substituents, independently selected from the group consisting of: halogen; lower alkyl optionally substituted with halogen, lower alkoxy or phenyl; lower alkoxy optionally substituted with halogen or lower alkoxy; cycloalkyl; cycloalkoxy; phenyl optionally substituted with halogen, cyano, lower alkyl, halo-lower alkyl, lower alkoxy or halo-lower alkoxy; heterocyclyl optionally substituted with halogen, cyano, lower alkyl, halo-lower alkyl, lower alkoxy or halo-lower alkoxy; and oxo.

The unsaturated heteromonocyclic ring is preferably a 5- or 6-membered unsaturated heterocyclic ring containing 1 or 2 hetero atoms independently selected from nitrogen, oxygen, and sulfur. More specifically, preferred examples of the unsaturated heteromonocyclic ring include furan, thiophene, oxazole, isoxazole, triazole, tetrazole, pyrazole, pyridine, pyrimidine, pyrazine, dihydroisoxazole, dihydropyridine, and thiazole. The unsaturated fused heterobicyclic ring is preferably a 9- or 10-membered unsaturated fused heterocyclic ring containing 1 to 4 hetero atoms independently selected from nitrogen, oxygen, and sulfur. More specifically, preferred examples of the unsaturated fused heterobicyclic ring are indoline, isoindoline, benzothiazole, benzoxazole, indole, indazole, quinoline, isoquinoline, benzothiophene, benzofuran, thienothiophene, and dihydroisoquinoline.

In a more preferable embodiment of the present invention, Ring A is

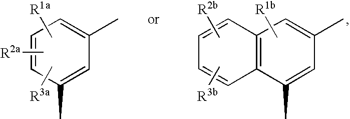

wherein R^{1a}, R^{2a}, R^{3a}, R^{1b}, R^{2b}, and R^{3b} are each independently hydrogen, halogen, hydroxy, alkoxy, alkyl, haloalkyl, haloalkoxy, hydroxyalkyl, alkoxyalkyl, alkoxyalkoxy, alkenyl, alkynyl, cycloalkyl, cycloalkenyl, cycloalkyloxy, phenyl, phenylalkoxy, cyano, nitro, amino, mono- or di-alkylamino, alkanoylamino, carboxyl, alkoxycarbonyl, carbamoyl, mono- or di-alkylcarbamoyl, alkanoyl, alkylsulfonylamino, phenylsulfonylamino, alkylsulfinyl, alkylsulfonyl, or phenylsulfonyl; Ring B is

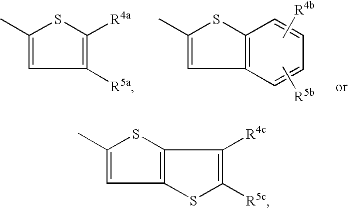

wherein R^{4a} and R^{5a} are each independently hydrogen; halogen; hydroxy; alkoxy; alkyl; haloalkyl; haloalkoxy; hydroxyalkyl; alkoxyalkyl; phenylalkyl; alkoxyalkoxy; hydroxyalkoxy; alkenyl; alkynyl; cycloalkyl; cycloalkenyl; cycloalkyloxy; phenyloxy; phenylalkoxy; cyano; nitro; amino; mono- or di-alkylamino; alkanoylamino; carboxyl; alkoxycarbonyl; carbamoyl; mono- or di-alkylcarbamoyl; alkanoyl; alkylsulfonylamino; phenylsulfonylamino; alkylsulfinyl; alkylsulfonyl; phenylsulfonyl; phenyl optionally substituted with 1-3 groups selected from halogen, cyano, alkyl, haloalkyl, alkoxy, haloalkoxy, alkylenedioxy, alkyleneoxy, mono- or di-alkylamino, carbamoyl, and mono- or di-alkylcarbamoyl; or heterocyclyl optionally substituted with 1-3 groups selected from halogen, cyano, alkyl, haloalkyl, alkoxy, haloalkoxy, mono- or di-alkylamino, carbamoyl, and mono- or di-alkylcarbamoyl, or R^{4a} and R^{5a} are bonded to each other at the terminals thereof to form alkylene;
R^{4b}, R^{5b}, R^{4c} and R^{5c} are each independently hydrogen; halogen; hydroxy; alkoxy; alkyl; haloalkyl; haloalkoxy; hydroxyalkyl; alkoxyalkyl; phenylalkyl; alkoxyalkoxy; hydroxyalkoxy; alkenyl; alkynyl; cycloalkyl; cycloalkenyl; cycloalkyloxy; phenyloxy; phenylalkoxy; cyano; nitro; amino; mono- or di-alkylamino; alkanoylamino; carboxyl; alkoxycarbonyl; carbamoyl; mono- or di-alkylcarbamoyl; alkanoyl; alkylsulfonylamino; phenylsulfonylamino; alkylsulfinyl; alkylsulfonyl; phenylsulfonyl; phenyl optionally substituted with 1-3 groups selected from halogen, cyano, alkyl, haloalkyl, alkoxy, haloalkoxy, alkylenedioxy, alkyleneoxy, and mono- or di-alkylamino; or heterocyclyl optionally substituted with 1-3 groups selected from halogen, cyano, alkyl, haloalkyl, alkoxy and haloalkoxy; and

### Z is:

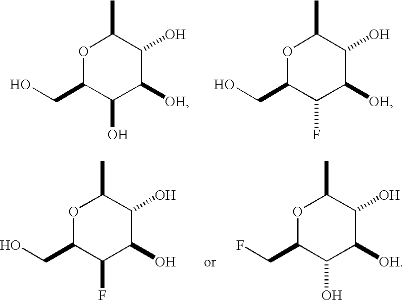

In a more preferable embodiment, R^{1a}, R^{2a}, R^{3a}, R^{1b}, R^{2b}, and R^{3b} are each independently hydrogen, halogen, lower alkyl, halo-lower alkyl, lower alkoxy, or phenyl; R^{4a} and R^{5a} are each independently hydrogen; halogen; lower alkyl; halo-lower alkyl; phenyl-lower alkyl; phenyl optionally substituted with 1-3 groups selected from halogen, cyano, lower alkyl, halo-lower alkyl, lower alkoxy, halo-lower alkoxy, methylenedioxy, ethyleneoxy, mono- or di-lower alkylamino, carbamoyl, and mono- or di-lower alkylcarbamoyl; or heterocyclyl optionally substituted with 1-3 groups selected from halogen, cyano, lower alkyl, halo-lower alkyl, lower alkoxy, halo-lower alkoxy, mono- or di-alkylamino, carbamoyl, and mono- or di-lower alkylcarbamoyl, or R^{4a} and R^{5a} are bonded to each other at the terminals thereof to form lower alkylene; and

### R^{4b}, R^{5b}, R^{4c} and R^{5c} are each independently hydrogen, halogen, lower alkyl, halo-lower alkyl, lower alkoxy, or halo-lower alkoxy.

In a further preferable embodiment, Ring A is

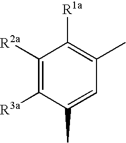

wherein R^{1a} is halogen, lower alkyl, or lower alkoxy, and R^{2a} and R^{3a} are hydrogen; Ring B is

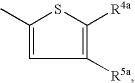

wherein R^{4a} is phenyl optionally substituted with 1-3 groups selected from halogen, cyano, lower alkyl, halo-lower alkyl, lower alkoxy, halo-lower alkoxy, methylenedioxy, ethyleneoxy, mono- or di-lower alkylamino, carbamoyl, and mono- or di-lower alkylcarbamoyl; or heterocyclyl optionally substituted with 1-3 groups selected from halogen, cyano, lower alkyl, lower alkoxy, mono- or di-alkylamino, carbamoyl, and mono- or di-lower alkylcarbamoyl, and R^{5a} is hydrogen; or R^{4a} and R^{5a} are bonded to each other at the terminals thereof to form lower alkylene; and Y is —CH₂—.

In this embodiment, R^{1a} is preferably halogen or lower alkyl; R^{4a} is preferably phenyl optionally substituted with 1-3 groups selected from halogen, cyano, lower alkyl, halo-lower alkyl, lower alkoxy, halo-lower alkoxy, mono- or di-lower alkylamino, carbamoyl, and mono- or di-lower alkylcarbamoyl; or heterocyclyl optionally substituted with 1-3 groups selected from halogen, cyano, lower alkyl, lower alkoxy, carbamoyl, and mono- or di-lower alkylcarbamoyl; and R^{5a} is hydrogen.

In this embodiment, R^{4a} is preferably phenyl optionally substituted with halogen, cyano, lower alkyl, halo-lower alkyl, lower alkoxy, halo-lower alkoxy, or mono- or di-lower alkylamino; or heterocyclyl optionally substituted with halogen, cyano, lower alkyl, halo-lower alkyl, lower alkoxy, or halo-lower alkoxy.

In a more preferable embodiment, R^{4a} is phenyl substituted with halogen, cyano, lower alkyl, halo-lower alkyl, lower alkoxy or halo-lower alkoxy; or heterocyclyl substituted with halogen, cyano, lower alkyl, or lower alkoxy.

In a further preferable embodiment, heterocyclyl is 5- or 6-membered heterocyclyl containing 1 or 2 hetero atoms independently selected from the group consisting of: nitrogen, oxygen, and sulfur, or 9- or 10-membered heterocyclyl containing 1 to 4 hetero atoms independently selected from the group consisting of: nitrogen, oxygen, and sulfur. Examples of heterocyclyl preferably include thienyl, pyridyl, pyrimidyl, pyrazinyl, pyrazolyl, thiazolyl, quinolyl, tetrazolyl and oxazolyl.

In a further preferable embodiment, R^{4a} is phenyl substituted with halogen or cyano, or pyridyl substituted with halogen.

In another preferable embodiment of the present invention, Ring A is

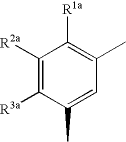

wherein R^{1a} is halogen, lower alkyl, or lower alkoxy, and R^{2a} and R^{3a} are both hydrogen; and Ring B is

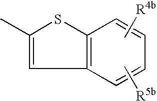

wherein R^{4b} and R^{5b} are each independently hydrogen, halogen, lower alkyl, halo-lower alkyl, lower alkoxy, or halo-lower alkoxy.

In still another preferable embodiment of the present invention, Ring A is:

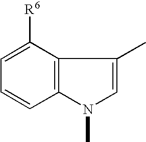

wherein R⁶ is hydrogen, halogen, or alkyl; Ring B is:

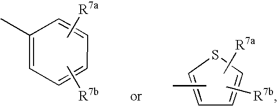

in which R^{7a} and R^{7b} are independently hydrogen, halogen, alkyl, cycloalkyl, haloalkyl, alkoxy, haloalkoxy, alkylthio, hydroxy, phenyl, halophenyl, cyanophenyl, pyridyl, halopyridyl, thienyl, or halothienyl, or R^{7a} and R^{7b} together with carbon atoms to which they are attached form a fused benzene, furan or dihydrofuran ring; and Y is CH₂.

In a more preferable embodiment, R⁶ is halogen, Z is:

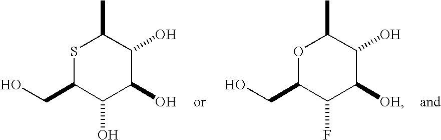

R^{7a} and R^{7b} are independently hydrogen, halogen, alkyl, cycloalkyl, haloalkyl, alkoxy, haloalkoxy, alkylthio, phenyl, halophenyl, cyanophenyl, pyridyl or halopyridyl, or R^{7a} and R^{7b} together with carbon atoms to which they are attached form a fused benzene, furan or dihydrofuran ring.

In a preferable embodiment, R^{7a} and R^{7b} are independently hydrogen, halogen, alkyl, cycloalkyl, haloalkyl, alkoxy, haloalkoxy, or alkylthio, or R^{7a} and R^{7b} together with carbon atoms to which they are attached form a fused furan or dihydrofuran ring.

In a more preferable embodiment, R^{7a} and R^{7b} are independently hydrogen, halogen, alkyl, cycloalkyl, haloalkyl, alkoxy, or haloalkoxy, or R^{7a} and R^{7b} together with carbon atoms to which they are attached form a fused furan or dihydrofuran ring.

In another preferable embodiment, R⁶ is fluorine, chlorine, or bromine, and preferably fluorine or chlorine.

In still another preferable embodiment, R⁶ is halogen, and Ring B is:

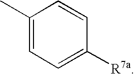

In this embodiment, R^{7a} is preferably halogen, alkyl, cycloalkyl, haloalkyl, alkoxy, haloalkoxy or alkylthio. More preferably, R^{7a} is halogen (e.g., chlorine, bromine, and iodine), alkyl (e.g., methyl and ethyl), cycloalkyl (e.g., cyclopropyl), haloalkyl (e.g., difluoromethyl and trifluoromethyl), alkoxy (e.g., methoxy, and ethoxy), or haloalkoxy (e.g., chloroethoxy, difluoromethoxy and trifluoromethoxy). Further preferably, R^{7a} is halogen, alkyl, cycloalkyl, or alkoxy.

In another preferable embodiment, Ring B is the following:

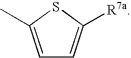

In this embodiment, preferably R⁶ is halogen, and R^{7a} is halogen (e.g., fluorine, and chlorine), or alkyl (e.g., methyl and ethyl).

In still another preferable embodiment, Ring B is

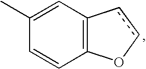

in which [symbol] represents a single bond or a double bond, and R⁶ is halogen.

In still another preferable embodiment, Ring A is indole, chloroindole, or chlorobenzene, Ring B is ethylphenyl, ethoxyphenyl, benzo[b]thiophen-2-yl, or 5-phenyl-2-thienyl, Y is —CH₂—, and Z is 5-thio-β-D-glucopyranosyl, 4-fluoro-4-deoxy-β-D-glucopyranosyl, 4-fluoro-4-deoxy-β-D-galactopyranosyl, or 6-fluoro-6-deoxy-β-D-glucopyranosyl.

Preferred compounds of the present invention may be selected from the following group:
3-(4-Ethylphenylmethyl)-1-(5-thio-β-D-glucopyranosyl)indole;
4-Chloro-3-(4-ethylphenylmethyl)-1-(5-thio-β-D-glucopyranosyl)indole;
4-Chloro-3-(4-ethylphenylmethyl)-1-(4-fluoro-4-deoxy-β-D-glucopyranosyl)indole;
4-Chloro-3-(4-ethoxyphenylmethyl)-1-(5-thio-β-D-glucopyranosyl)indole;
3-(Benzo[b]thiophen-2-ylmethyl)-4-chloro-1-(4-fluoro-4-deoxy-β-D-galactopyranosyl)benzene;
4-Chloro-3-(5-phenyl-2-thienylmethyl)-1-(6-fluoro-6-deoxy-β-D-glucopyranosyl)benzene;
4-Chloro-3-(5-phenyl-2-thienylmethyl)-1-(4-fluoro-4-deoxy-β-D-glucopyranosyl)benzene; and
   a pharmaceutically acceptable salt thereof.

The compounds of the present invention possess activity as inhibitors of sodium-dependent glucose transporter, and show excellent blood glucose lowering effect.

The compounds of the present invention are expected to be useful in the treatment, prevention or delaying the progression or onset of diabetes mellitus (type 1 and type 2 diabetes mellitus, etc.), diabetic complications (such as diabetic retinopathy, diabetic neuropathy, diabetic nephropathy), postprandial hyperglycemia, delayed wound healing, insulin resistance, hyperglycemia, hyperinsulinemia, elevated blood levels of fatty acids, elevated blood levels of glycerol, hyperlipidemia, obesity, hypertriglyceridemia, Syndrome X, atherosclerosis, or hypertension.

The compounds of the present invention or a pharmaceutically acceptable salt thereof may be administered either orally or parenterally, and can be used in the form of a suitable pharmaceutical preparation. Suitable pharmaceutical preparations for oral administration include, for example, solid preparations such as tablets, granules, capsules, and powders, or solution preparations, suspension preparations, emulsion preparations, and the like. Suitable pharmaceutical preparations for parenteral administration include, for example, suppositories; injection preparations or intravenous drip preparations, using distilled water for injection, physiological saline solution or aqueous glucose solution; and inhalant preparations.

The pharmaceutical compositions herein will contain, per dosage unit, e.g., tablet, capsule, powder, injection, suppository, teaspoonful and the like, from about 0.01 mg/kg to about 100 mg/kg body weight (preferably from about 0.01 mg/kg to about 50 mg/kg; and, more preferably, from about 0.01 mg/kg to about 30 mg/kg) of the active ingredient, and may be given at a dosage of from about 0.01 mg/kg/day to about 100 mg/kg/day (preferably from about 0.01 mg/kg/day to about 50 mg/kg/day and more preferably from about 0.01 mg/kg/day to about 30 mg/kg/day). The method of treating a disorder described in the present invention may also be carried out using a pharmaceutical composition comprising any of the compounds as defined herein and a pharmaceutical acceptable carrier. The dosage form will contain from about 0.01 mg/kg to about 100 mg/kg (preferably from about 0.01 mg/kg to about 50 mg/kg; and, more preferably, from about 0.01 mg/kg to about 30 mg/kg) of the active ingredient, and may be constituted into any form suitable for the mode of administration selected. The dosages, however, may be varied depending upon administration routes, the requirement of the subjects, the severity of the condition being treated and the compound being employed. The use of either daily administration or post-periodic dosing may be employed.

The compounds of formula (A) may be used, if necessary, in combination with one or more of other anti-diabetic agents, antihyperglycemic agents and/or agents for treatment of other diseases. The present compounds and these other agents may be administered in the same dosage form, or in a separate oral dosage form or by injection.

Examples of the other anti-diabetic agents and anti-hyper glycemic agents include insulin, insulin secretagogues, insulin sensitizers, or other antidiabetic agents having an action mechanism different from SGLT inhibition. Specifically, examples of these agents are biguanides, sulfonylureas, α-glucosidase inhibitors, PPARγ agonists (e.g., thiazolidinedione compounds), PPARα/γ dual agonists, PPARpan agonists, dipeptidyl peptidase IV (DPP4) inhibitors, mitiglinide, nateglinide, repaglinide, insulin, glucagon-like peptide-1 (GLP-1) and its receptor agonists, PTP1B inhibitors, glycogen phosphorylase inhibitors, RXR modulators, glucose 6-phosphatase inhibitors, GPR40 agonists/antagonists, GPR119 agonists, GPR120 agonists, glucokinase (GK) activators, and fructose 1,6-bisphosphatase (FBPase) inhibitors.

Examples of the agents for treatment of other diseases include anti-obesity agents, antihypertensive agents, antiplatelet agents, anti-atherosclerotic agents and hypolipidemic agents.

The anti-obesity agents which may be optionally employed in combination with the compound of the present invention include β₃ adrenergic agonists, lipase inhibitors, serotonin (and dopamine) reuptake inhibitors, thyroid hormone receptor beta drugs, anorectic agents, NPY antagonists, Leptin analogs MC4 agonists and CB1 antagonists.

The anti-platelet agents which may be optionally employed in combination with the compound of the present invention include abciximab, ticlopidine, eptifibatide, dipyridamole, aspirin, anagrelide, tirofiban and clopidogrel.

The anti-hypertensive agents which may be optionally employed in combination with the compound of the present invention include ACE inhibitors, calcium antagonists, alpha-blockers, diuretics, centrally acting agents, angiotensin-II antagonists, beta-blockers, renin inhibitors, and vasopeptidase inhibitors.

The hypolipidemic agents which may be optionally employed in combination with the compound of the present invention include MTP inhibitors, HMG CoA reductase inhibitors, squalene synthetase inhibitors, squalene epoxidase inhibitors, fibric acid derivatives, ACAT inhibitors, lipoxygenase inhibitors, cholesterol absorption inhibitors, ileal Na⁺/bile acid cotransporter inhibitors, upregulators of LDL receptor activity, bile acid sequestrants, nicotinic acid and derivatives thereof, CETP inhibitors, and ABC A1 upregulators.

The compounds of formula (A) may be used in combination with agents for treatment of diabetic complications, if necessary. These agents include, for example, PKC inhibitors and/or ACE inhibitors.

The various agents described above may be employed in the same dosage form with compounds of formula (A) or in different dosage forms, in dosages and regimens as generally known in the art.

The dosage of those agents may vary according to, for example, ages, body weight, conditions of patients, administration routes, and dosage forms.

These pharmaceutical compositions may be orally administered to mammalian species including human beings, apes, and dogs, in the dosage form of, for example, tablet, capsule, granule or powder, or parenterally administered in the form of injection preparation, or intranasally, or in the form of transdermal patch.

The compounds of formula (A) of the present invention or a pharmaceutically acceptable salt thereof, can be prepared in accordance with one of the following Schemes.

During any of the processes for preparation of the compounds of the present invention, it may be necessary and/or desirable to protect sensitive or reactive groups on any of the molecules concerned. This may be achieved by means of conventional protecting groups. For a general description of protecting groups and their use, see T. W. Greene et al., “Protecting Groups in Organic Synthesis”, John Wiley & Sons, New York, 1999. The protecting groups may be removed at a subsequent step using methods known to those skilled in the art.

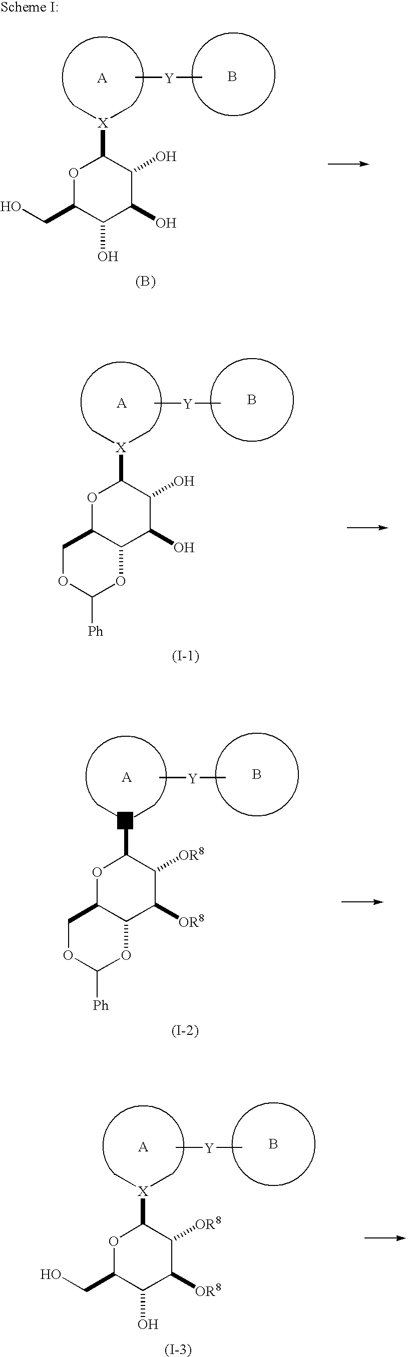

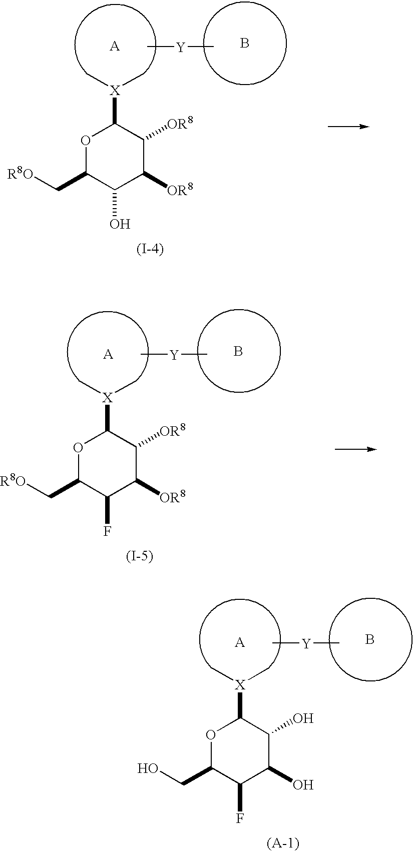

### (In the above scheme, OR⁸ is a protected hydroxy group, Ph is phenyl, and the other symbols are the same as defined above.)

### Step I-1:

The compounds of formula (I-1) can be prepared by condensing the compounds of formula (B) with benzaldehyde dimethyl acetal. The condensation reaction can be carried out by conventional methods well known to those skilled in the art. The condensation is typically carried out in a suitable solvent such as ethers (e.g., diethyl ether, tetrahydrofuran, and 1,4-dioxane), halogenoalkanes (e.g., dichloromethane, chloroform, and 1,2-dichloroethane), and a mixture of these solvents, in the presence of an acid such as hydrochloric acid, sulfuric acid, tetrafluoroboric acid, and p-toluenesulfonic acid.

### Step I-2:

The compounds of formula (I-2) can be prepared by protecting hydroxy groups of the compounds of formula (I-1). The protecting group for the hydroxy groups can be selected from those conventionally used as protecting groups for hydroxy. Examples of the protecting group for a hydroxy group include alkanoyl (e.g., acetyl), arylcarbonyl (e.g., benzoyl), arylalkyl (e.g., benzyl, tolyl, and anisyl), alkylsilyl (e.g., trimethylsilyl, t-butyldimethylsilyl, and triethylsilyl). Preferably, R⁸ is alkanoyl such as acetyl or benzoyl. The protection can be carried out by conventional methods well known to those skilled in the art. For a general description of protecting groups and their use, see T. W. Greene et al., “Protecting Groups in Organic Synthesis”, John Wiley & Sons, New York, 1999.

### Step I-3:

The compounds of formula (I-3) can be prepared by hydrolysis of the compounds of formula (I-2). The hydrolysis is typically carried out in the presence of an acid (e.g., hydrochloric acid, acetic acid, and sulfuric acid) in a suitable solvent (e.g., methanol, ethyl alcohol, water and a mixture of these solvents) or without a solvent.

### Step I-4:

The compounds of formula (I-4) can be prepared by protecting the compounds of formula (I-3). The protection can be carried out in accordance with Step I-2.

### Step I-5:

The compounds of formula (I-5) can be prepared by fluorination of the compounds of formula (I-4). The fluorination is typically carried out by reacting the compounds of formula (I-4) with a fluorinating agent such as (diethylamino)sulfur trifluoride in a suitable solvent such as halogenoalkanes (e.g., dichloromethane, chloroform) and ethers (diethyl ether, tetrahydrofuran).

### Step I-6:

The compounds of formula (A-1) can be prepared by deprotecting the compounds of formula (I-5), followed by converting the resulting compounds into a pharmaceutically acceptable salt, if desired.

The deprotection can be carried out according to kinds of the protecting group to be removed, and conventional methods such as reduction, hydrolysis, acid treatment, and fluoride treatment, can be used for the deprotection.

When a protecting group is to be removed by hydrolysis, the hydrolysis can be carried out by treating the compounds of formula (I-5) with a base (e.g., sodium hydroxide, potassium hydroxide, lithium hydroxide, sodium methoxide, and sodium ethoxide) in a suitable inert solvent (e.g., tetrahydrofuran, dioxane, methanol, ethyl alcohol, and water).

The deprotection reaction can be preferably carried out at lowered, ambient or elevated temperature, for example, from 0° C. to 50° C., more preferably from 0° C. to room temperature.

The compounds of formula (I-5) are believed to be novel and form a further aspect of this invention.

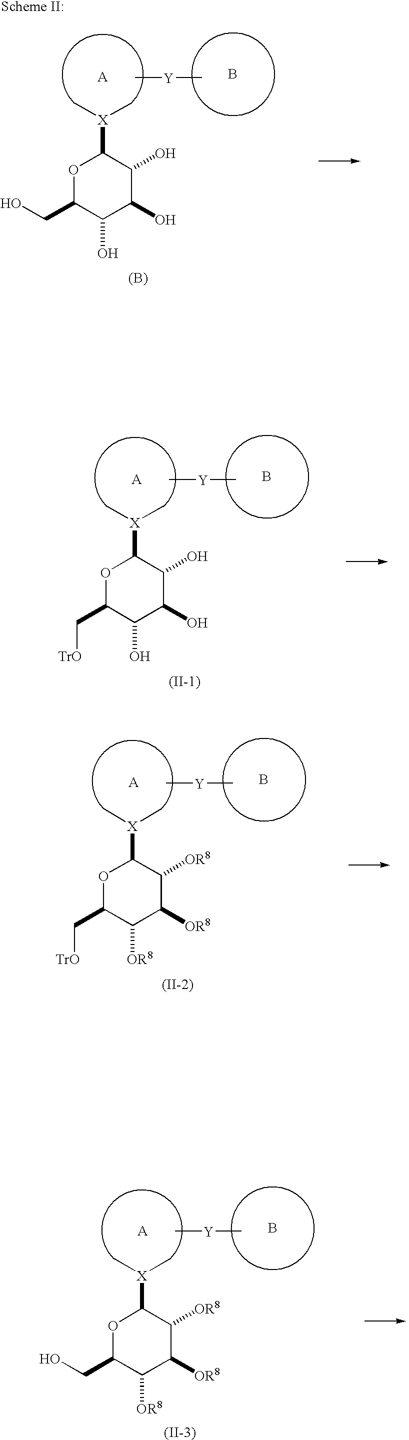

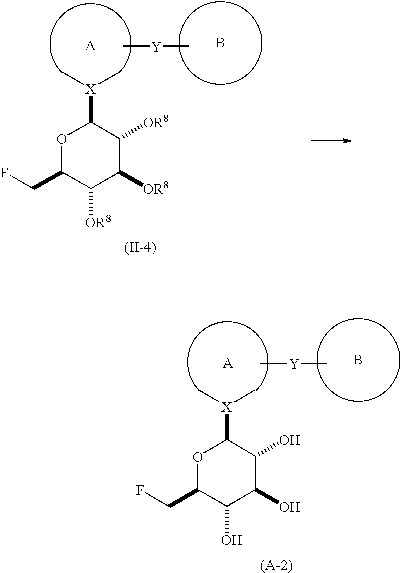

### In the above scheme, Tr is trityl (i.e., triphenylmethyl), and the other symbols are the same as defined above.

### Step II-1:

The compounds of formula (II-1) can be prepared by reacting the compounds of formula (B) with trityl chloride. This reaction is typically carried out in the presence of a base (e.g., pyridines such as pyridine and N,N-dimethylaminopyridine; alkylamines such as triethylamine and diisopropylethylamine; inorganic bases such as NaHCO₃ and K₂CO₃) in a suitable solvent (e.g., amides such as dimethylformamide, and N-methylpyrrolidone; ethers such as tetrahydrofuran; halogenoalkanes such as dichloromethane; and a mixture of these solvents) at lowered, ambient or elevated temperature, for example, from 0° C. to 50° C., more preferably from 0° C. to room temperature.

### Step II-2:

The compounds of formula (II-2) can be prepared by protecting hydroxy groups of the compounds of formula (II-1). The protection can be carried out in accordance with Step I-2.

### Step II-3:

The compounds of formula (II-3) can be prepared by hydrolysis of the compounds of formula (II-2). The hydrolysis is typically carried out in the presence of an acid (e.g., acetic acid, formic acid, hydrochloric acid, and sulfuric acid) in a suitable solvent (e.g., diethyl ether, tetrahydrofuran, methanol, and ethyl alcohol, water and a mixture of these solvents) or without a solvent at lowered, ambient or elevated temperature, for example, from 0° C. to 50° C., more preferably from 0° C. to room temperature.

### Step II-4:

The compounds of formula (II-4) can be prepared by fluorination of the compounds of formula (II-3). The fluorination can be carried out in accordance with Step I-5.

### Step II-5:

The compounds of formula (A-2) can be prepared by deprotecting compounds of formula (II-4), followed by converting the resulting compound into a pharmaceutically acceptable salt, if desired.

The deprotection can be carried out in accordance with Step I-6.

The compounds of formula (II-4) are believed to be novel and form a further aspect of this invention.

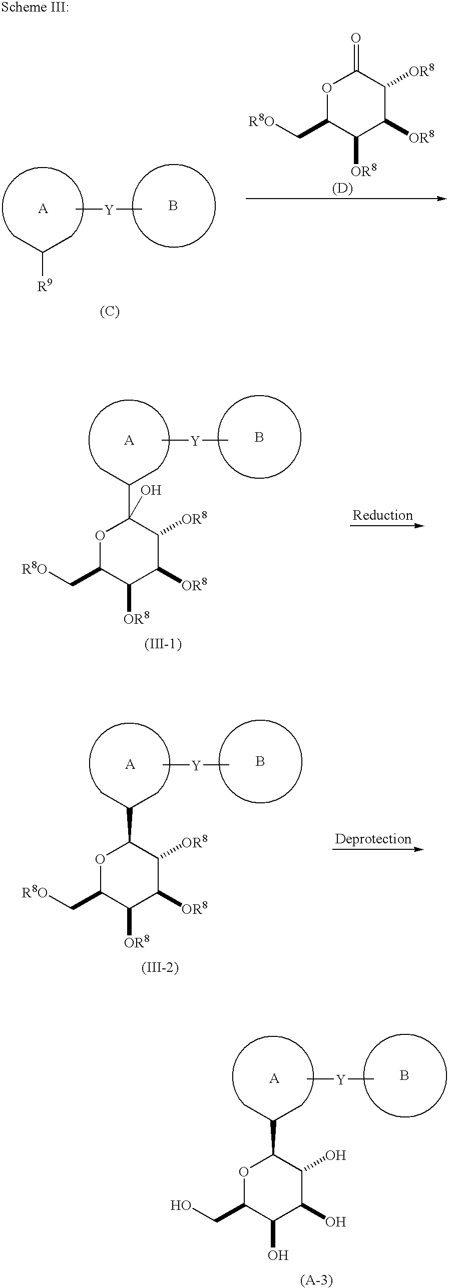

### In the above scheme, R⁹ is bromine or iodine, and the other symbols are the same as defined above.

### Step III-1:

The compounds of formula (III-1) can be prepared by lithiating the compounds of formula (C) with an alkyllithium (e.g., methyl lithium, n-butyl lithium, t-butyl lithium) in a suitable solvent (e.g., ethers such as tetrahydrofuran, diethyl ether) at lowered temperature (for example, from −78° C. to 0° C.), followed by reacting the resultant with the compounds of formula (D) in the solvent at lowered or ambient temperature, for example, from −78° C. to room temperature.

### Step III-2:

The compound of formula (III-2) can be prepared by reducing the compounds of formula (III-1). The reduction is typically carried out in the presence of a reducing agent (e.g., a silane reagent such as triethylsilane) and a Lewis acid (e.g., boron trifluoride.diethyl ether complex) in a suitable solvent (e.g., acetonitrile, dichloromethane, etc.) at lowered or ambient temperature, for example, from −78° C. to room temperature.

### Step III-3:

The compounds of formula (A-3) can be prepared by deprotecting the compounds of formula (III-2).

The deprotection is typically carried out in accordance with Step I-6.

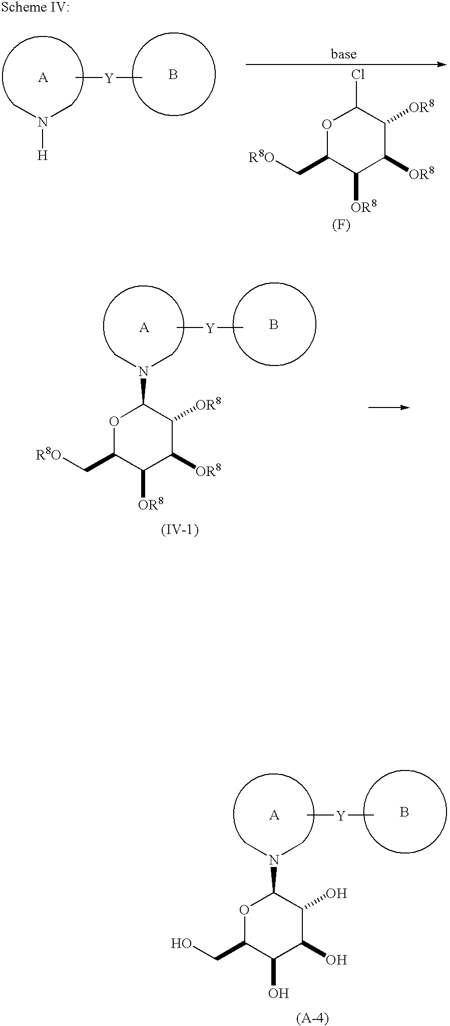

### In the above scheme, the symbols are the same as defined above.

### Step IV-1

The compounds of formula (IV-1) can be prepared by condensing the compounds of formula (E) with the compounds of formula (F) (for example, 2,3,4,6-tetra-O-benzyl-D-galactopyranosyl chloride) in a suitable solvent in the presence of a base.

The condensation reaction of the compounds of formula (E) with the compounds of formula (F) can be carried out in a suitable solvent (e.g., ethers such as tetrahydrofuran and diethyl ether and amides such as dimethyl formamide) in the presence of a base (e.g., sodium hydride and potassium hydroxide) at lower, ambient or elevated temperature, for example, from 0° C. to 100° C.

### Step IV-2:

The compounds of formula (A-4) can be prepared by deprotecting the compounds of formula (IV-1). The deprotection can be carried out in accordance with Step I-6.

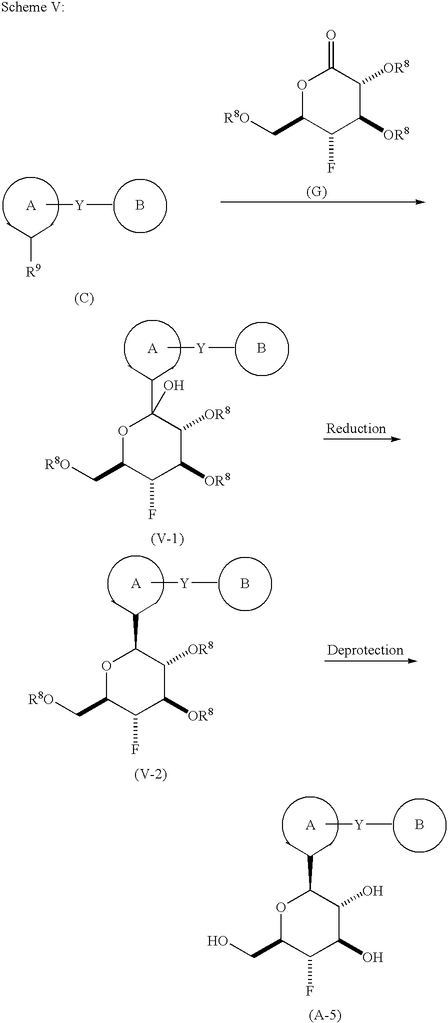

### In the above scheme, the symbols are the same as defined above.

### Step V-1:

The compounds of formula (V-1) can be prepared by lithiating the compounds of formula (C) with an alkyllithium in a suitable solvent at lowered temperature, followed by reacting the resultant with the compounds of formula (G) in the solvent at lowered or ambient temperature. This step can be carried out in accordance with Step III-1.

### Step V-2:

The compounds of formula (V-2) can be prepared by reducing the compounds of formula (V-1). The reduction can be carried out in accordance with Step III-2.

### Step V-3:

The compounds of formula (A-5) can be prepared by deprotecting the compounds of formula (V-2). The deprotection is typically carried out in accordance with Step I-6.

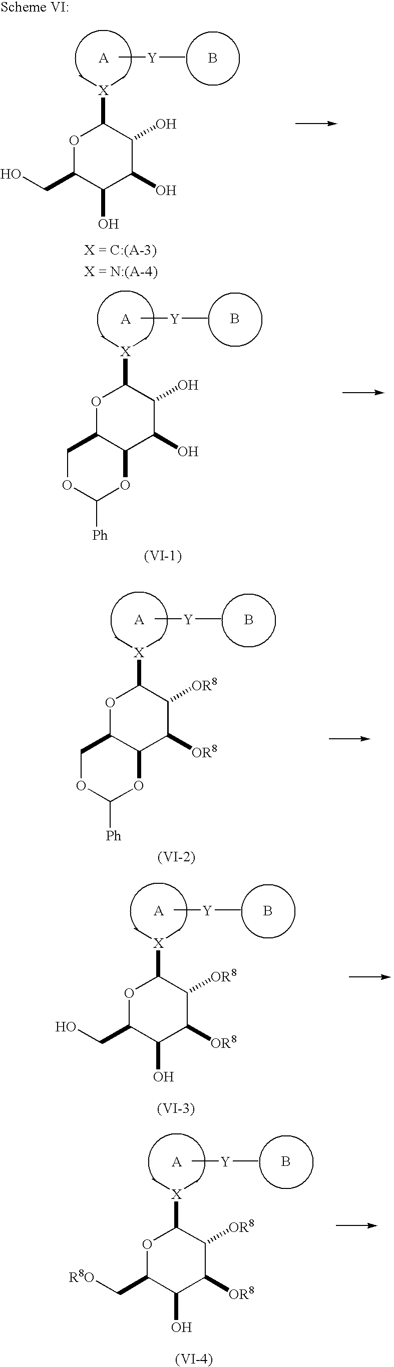

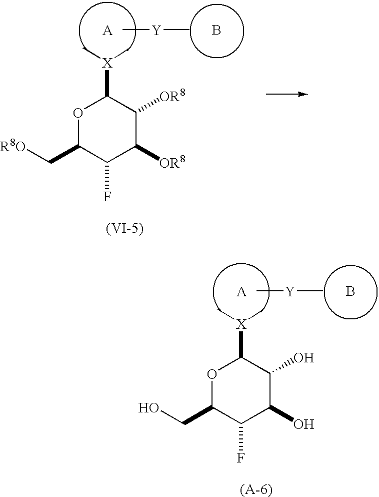

### In the above scheme, the symbols are the same as defined above.

### Step VI-1:

The compounds of formula (VI-1) can be prepared by condensing the compounds of formula (A-3) or (A-4) with benzaldehyde dimethyl acetal. The condensation reaction can be carried out in accordance with Step I-1.

### Step VI-2:

The compounds of formula (VI-2) can be prepared by protecting hydroxy groups of the compounds of formula (VI-1). The protection can be carried out in accordance with Step I-2.

### Step VI-3:

The compounds of formula (VI-3) can be prepared by hydrolysis of the compounds of formula (VI-2). The hydrolysis can be carried out in accordance with Step I-3.

### Step VI-4:

The compounds of formula (VI-4) can be prepared by protecting the compounds of formula (VI-3). The protection can be carried out in accordance with Step I-2.

### Step VI-5:

The compounds of formula (VI-5) can be prepared by fluorination of the compounds of formula (VI-4). The fluorination can be carried out in accordance with Step I-5.

### Step VI-6:

The compounds of formula (A-6) can be prepared by deprotecting compounds of formula (VI-5), followed by converting the resulting compound into a pharmaceutically acceptable salt, if desired.

The deprotection can be carried out in accordance with Step I-6.

The compounds of formula (VI-5) are believed to be novel and form a further aspect of this invention.

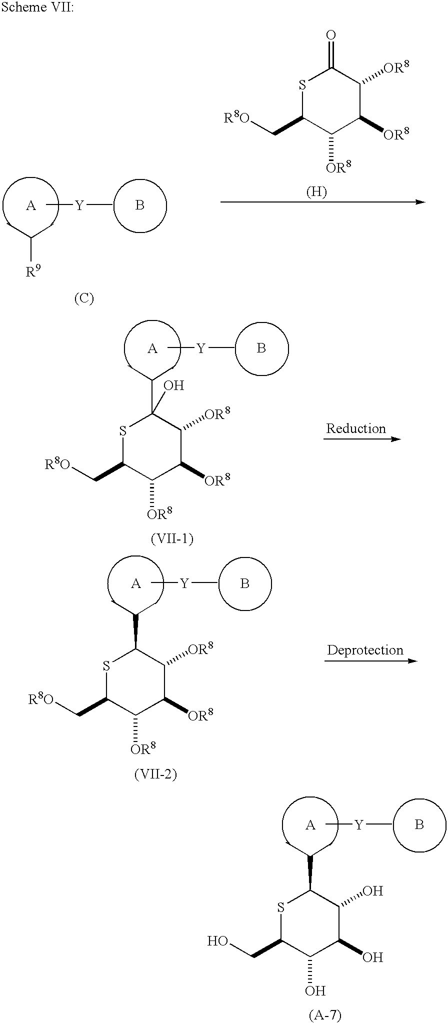

### In the above scheme, the symbols are the same as defined above.

### Step VII-1:

The compounds of formula (VII-1) can be prepared by lithiating the compounds of formula (C), reacting the lithiated compounds with CuI, and then, reacting the resultant with the compounds of formula (H) in the solvent at lowered or ambient temperature.

The lithiation of the compounds of formula (C) can be carried out by using an alkyllithium (e.g., methyl lithium, n-butyl lithium, t-butyl lithium) in a suitable solvent (e.g., ethers such as tetrahydrofuran, diethyl ether) at lowered temperature (for example, from −78° C. to 0° C.). The reaction of the lithiated compounds with CuI can be carried out in a suitable solvent (e.g., ethers such as tetrahydrofuran, diethyl ether) at lowered temperature (for example, from −78° C. to 0° C.). The reaction of the resultant with the compounds of formula (H) can be carried out in a suitable solvent (e.g., ethers such as tetrahydrofuran, diethyl ether) at lowered or ambient temperature (for example, from −78° C. to room temperature).

### Step VII-2:

The compounds of formula (VII-2) can be prepared by reducing the compounds of formula (VII-1). The reduction can be carried out in accordance with the Step III-2.

### Step VII-3:

The compounds of formula (A-7) can be prepared by deprotecting the compounds of formula (VII-2).

The deprotection is typically carried out in accordance with Step I-6.

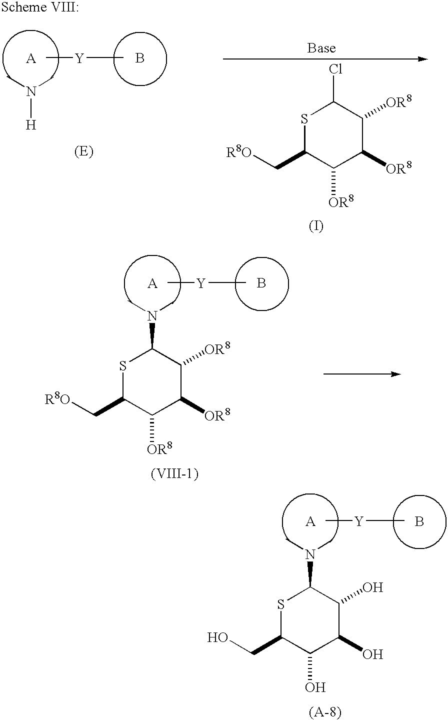

### In the above scheme, the symbols are the same as defined above.

### Step VIII-1:

The compounds of formula (VIII-1) can be prepared by condensing the compounds of formula (E) with the compounds of formula (I) (see Graeme D., et al., Tetrahedron Lett., 2001, 42, 1197-1200) in a suitable solvent in the presence of a base. This step can be carried out in accordance with Step IV-1.

### Step VIII-2:

The compounds of formula (A-8) can be prepared by deprotecting the compounds of formula (VIII-1). The deprotection can be carried out in accordance with Step I-6.

The compounds of formula (A) wherein Ring A is:

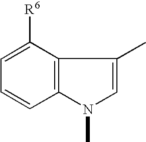

in which R⁶ is the same as defined above, Ring B is:

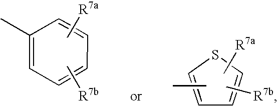

in which R^{7a} and R^{7b} are the same as defined above; and Y is CH₂, can be typically prepared in accordance with one of the following schemes:

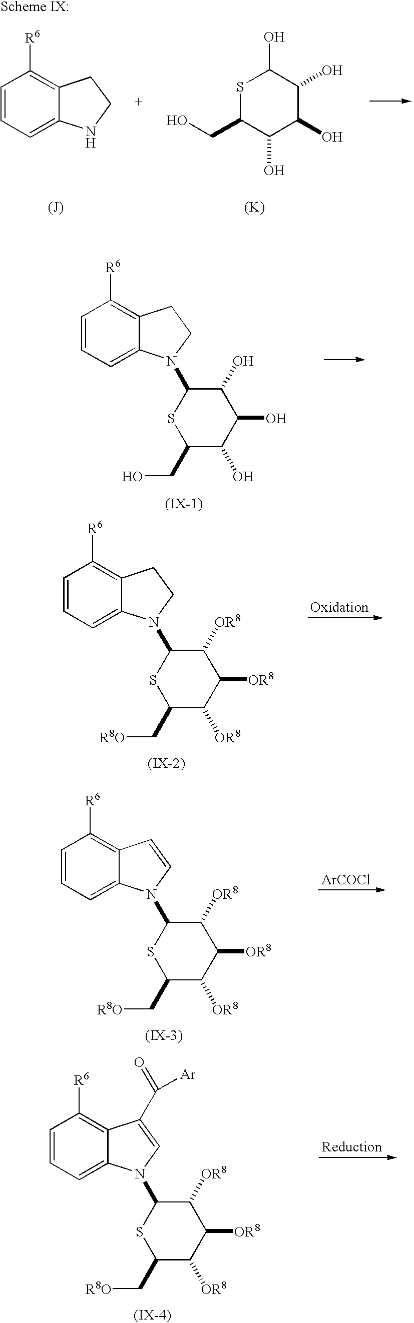

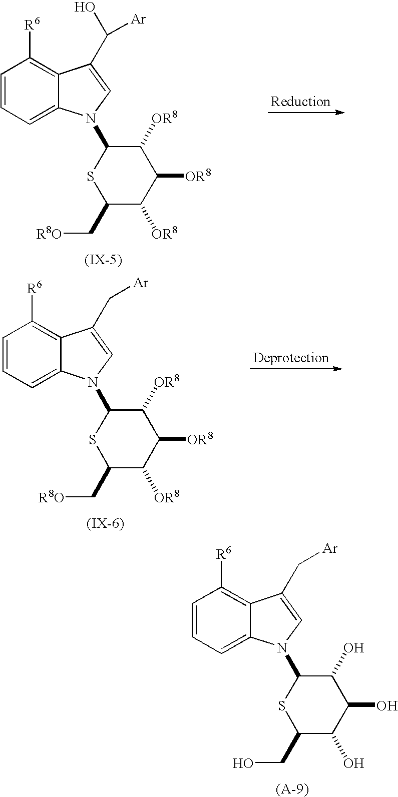

### In the above scheme, Ar is

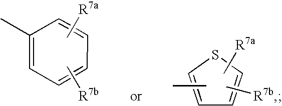

and R⁶, R^{7a}, R^{7b}, and R⁸ are the same as defined above.

### Step IX-1:

The compounds of formula (IX-1) can be prepared by condensing the compounds of formula (J) with the compounds of formula (K), or 5-thio-D-glucose. The condensation reaction is typically carried out in a suitable solvent such as acetonitrile, water and alcohols (e.g., methanol, ethyl alcohol and 1-propanol) with or without catalysts such as ammonium chloride and acetic acid at ambient or elevated temperature.

### Step IX-2:

The compounds of formula (IX-2) can be prepared by protecting hydroxy groups of the compounds of formula (IX-1). The protection can be carried out in accordance with Step I-2.

### Step IX-3:

The compounds of formula (IX-3) can be prepared by oxidation of the compounds of formula (IX-2). The oxidation reaction can be typically carried out in the presence of a oxidizing reagent such as palladium on charcoal, tetrachloro-1,4-benzoquinone (chloranil), 2,3-dichloro-5,6-dicyano-1,4-benzoquinone (DDQ) or ethylenebis(salicylimine)cobalt(II) salt in a suitable solvent such as ethers (e.g., diethyl ether, tetrahydrofuran, and 1,4-dioxane), halogenoalkanes (e.g., dichloromethane, chloroform, and 1,2-dichloroethane), water and a mixture of these solvents at ambient or lowered temperature.

### Step IX-4:

The compounds of formula (IX-4) can be prepared by condensing the compounds of formula (IX-3) with Ar—COCl wherein Ar is the same as defined above.

The condensation can be carried out, according to the Friedel-Crafts acylation well known in the art, in a suitable solvent in the presence of a Lewis acid.

Examples of the Lewis acid include aluminum chloride, boron trifluoride.diethyl ether complex, tin(IV) chloride, and titanium tetrachloride.

The solvent can be selected from any one which does not disturb the Friedel-Crafts reaction, and examples of the solvent include halogenoalkanes such as dichloromethane, chloroform, and dichloroethane.

The reaction can be carried out at lowered, ambient or elevated temperature, for example, from −30° C. to 60° C.

### Step IX-5:

The compounds of formula (IX-5) can be prepared by reducing the compounds of formula (IX-4).

The reduction can be carried out by treating the compounds (IX-4) with a reducing agent in a suitable solvent.

Examples of the reducing agent include borohydrides (e.g., sodium borohydride with or without cerium(III) chloride heptahydrate, sodium triacetoxyborohydride) and aluminum hydrides (e.g., lithium aluminum hydride, and diisobutyl aluminum hydride).

The solvent can be selected from any one which does not disturb the reaction and examples of the solvent include ethers (e.g., tetrahydrofuran, diethyl ether, dimethoxyethane, and dioxane), alcohols (e.g., methanol, ethyl alcohol and 2-propanol) and a mixture of these solvents.

The reduction reaction can be carried out at lowered, or ambient temperature, for example, from −30° C. to 25° C.

### Step IX-6:

The compounds of formula (IX-6) can be prepared by reducing the compounds of formula (IX-5).

The reduction of the compounds (IX-5) can be carried out by treatment with a silane reagent or a borohydride in the presence of an acid in a suitable solvent or without a solvent.

Examples of the acid include a Lewis acid such as boron trifluoride.diethyl ether complex and titanium tetrachloride, and a strong organic acid such as trifluoroacetic acid, and methanesulfonic acid.

Examples of silane reagents include trialkylsilanes such as triethylsilane, triisopropylsilane.

Examples of borohydrides include sodium borohydride and sodium triacetoxyborohydride.

The solvent can be selected from any one which does not disturb the reaction, and examples of the solvent include acetonitrile, halogenoalkanes (e.g., dichloromethane, chloroform and dichloroethane), and a mixture of these solvents.

The reduction can be carried out at lowered or ambient temperature, for example, from −30° C. to 25° C.

### Step IX-7:

The compounds of formula (A-9) or a pharmaceutically acceptable salt thereof, can be prepared by deprotecting compounds of formula (IX-6), followed by converting the resulting compound into a pharmaceutically acceptable salt, if desired.

The deprotection can be carried out in accordance with Step I-6.

The compounds of formula (IX-6) are believed to be novel and form a further aspect of this invention.

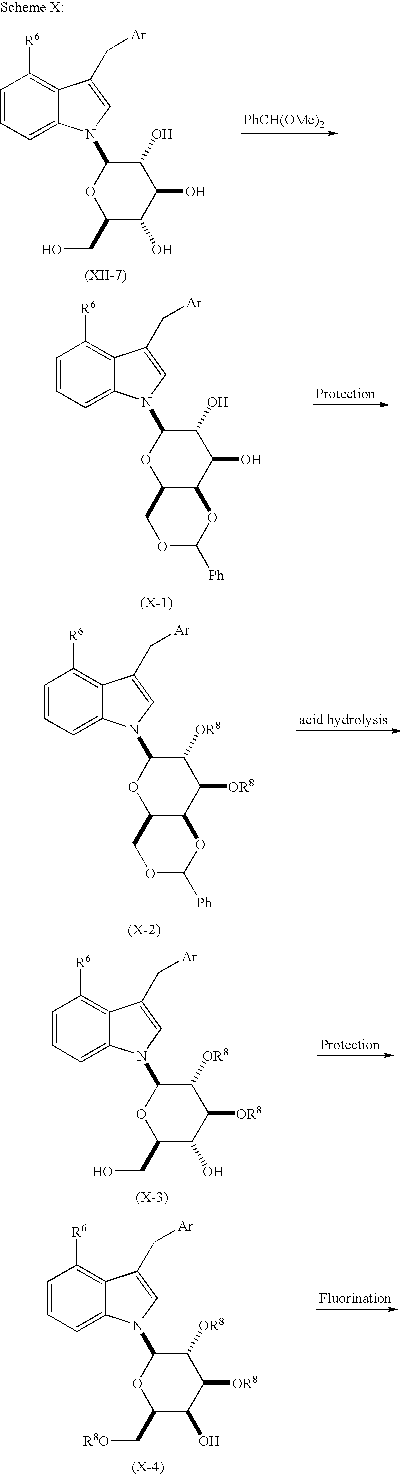

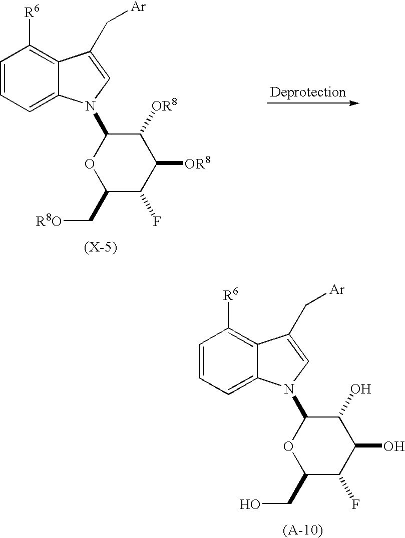

### In the above scheme, the symbols are the same as defined above.

### Step X-1:

The compounds of formula (X-1) can be prepared by condensing the compounds of formula (XII-7), described in Scheme XII below, with benzaldehyde dimethyl acetal. The condensation reaction can be carried out in accordance with Step I-1.

### Step X-2:

The compounds of formula (X-2) can be prepared by protecting hydroxy groups of the compounds of formula (X-1). The protection can be carried out in accordance with Step I-2.

### Step X-3:

The compounds of formula (X-3) can be prepared by hydrolysis of the compounds of formula (X-2). The hydrolysis can be carried out in accordance with Step I-3.

### Step X-4:

The compounds of formula (X-4) can be prepared by protecting the compounds of formula (X-3). The protection can be carried out in accordance with Step I-2.

### Step X-5:

The compounds of formula (X-5) can be prepared by fluorination of the compounds of formula (X-4). The fluorination can be typically carried out in accordance with Step I-5.

### Step X-6:

The compounds of formula (A-10) can be prepared by deprotecting compounds of formula (X-5), followed by converting the resulting compound into a pharmaceutically acceptable salt, if desired.

This step can be carried out in accordance with Step I-6.

The compounds of formula (X-5) are believed to be novel and form a further aspect of this invention.

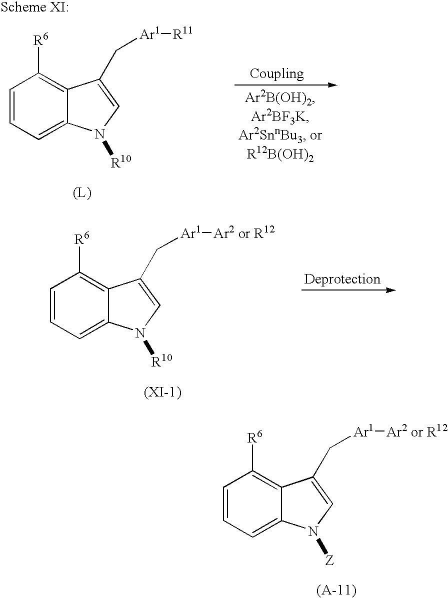

(In the above scheme, Ar¹ is phenyl, or thienyl, R¹¹ is bromine or iodine, Ar² is phenyl, halophenyl, cyanophenyl, pyridyl, halopyridyl, thienyl or halothienyl, R¹² is cycloalkyl, ⁿBu is n-butyl, R¹⁰ is one of the following groups:

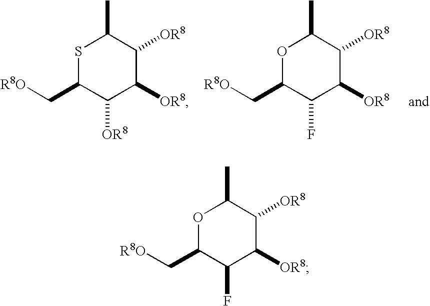

where OR⁸ is a protected hydroxy group, and R⁶ and Z are the same as defined above.)

### Step XI-1:

The compounds of formula (XI-1) can be prepared by coupling the compounds of formula (L) with Ar²B(OH)₂, Ar²BF₃K, Ar²SnⁿBu₃ or R¹²B(OH)₂, wherein Ar², R¹² and ⁿBu are as defined above.

The coupling reaction can be carried out by a conventional aryl coupling method, e.g., Suzuki coupling method (for reference see: Suzuki et al., Synth. Commun. 11:513 (1981); Suzuki, Pure and Appl. Chem. 57:1749-1758 (1985); Suzuki et al., Chem. Rev. 95:2457-2483 (1995); Shieh et al., J. Org. Chem. 57:379-381 (1992); Martin et al., Acta Chemica Scandinavica 47:221-230 (1993); Wallace et al., Tetrahedron Lett. 43:6987-6990 (2002) and Molander et al., J. Org. Chem. 68:4302-4314 (2003)) and Stille coupling method (for reference see: Stille, Angew. Chem. Int. Ed. Engl. 25:508-524 (1986) and Liebeskind et al., J. Org. Chem. 59:5905-5911 (1994)).

The coupling reaction can be carried out in the presence of a Pd catalyst and a base with or without a ligand and an additive in a suitable solvent.

Examples of the Pd catalyst include tetrakis(triphenyl-phosphine)palladium(0), palladium(II) acetate, bis(acetonitrile)dichloropalladium(II), dichlorobis(triphenylphosphine)palladium(II), [1,1′-bis(diphenylphosphino)ferrocene]dichloropalladium(II) complex with dichloromethane, tris(dibenzylidene-acetone)dipalladium(0)-chloroform adduct and palladium(II) chloride. Examples of the base include alkali metal carbonates (e.g., potassium carbonate, sodium carbonate and sodium bicarbonate), alkali metal phosphates (e.g., potassium phosphate tribasic, sodium phosphate and sodium hydrogenphosphate), organic bases (e.g., N,N-diisopropylethylamine) and alkali metal fluorides (e.g., cesium fluoride and potassium fluoride). Examples of the ligand include tricyclohexylphosphine and tri(o-tolyl)phosphine. Examples of the additive include copper(I) iodide.

The solvent can be selected from any one which does not disturb the coupling reaction, and examples of the solvent are aromatic hydrocarbons (e.g., benzene, and toluene), ethers (e.g., tetrahydrofuran, 1,2-dimethoxyethane, and 1,4-dioxane), amides (e.g., dimethylformamide, dimethylacetamide, 1,3-dimethyl-2-imidazolidinone and N-methylpyrrolidone), alcohols (methanol, ethyl alcohol, and 2-propanol), water, and a mixture of these solvents.

The coupling reaction can be carried out at ambient or elevated temperature, for example, from 25° C. to 150° C., preferably from 80° C. to 150° C.

### Step XI-2:

The compounds of formula (A-11) or a pharmaceutically acceptable salt thereof, can be prepared by deprotecting compounds of formula (XI-1), followed by converting the resulting compounds into a pharmaceutically acceptable salt, if desired. This step can be carried out in accordance with Step I-6.

The compounds of the present invention thus obtained may be isolated and purified by a conventional method well known in the organic synthetic chemistry such as recrystallization, column chromatography, thin layer chromatography, and the like.

The starting compounds of formula (B) are described in WO 2004/080990 pamphlet or WO 2005/012326 pamphlet, and thus, the compounds of formula (B) can be prepared in accordance with the procedures described in these pamphlets.

The compounds of formula (C) or formula (E) are disclosed in WO 2004/080990 pamphlet or WO 2005/012326 pamphlet and can be prepared in accordance with the procedures described therein.

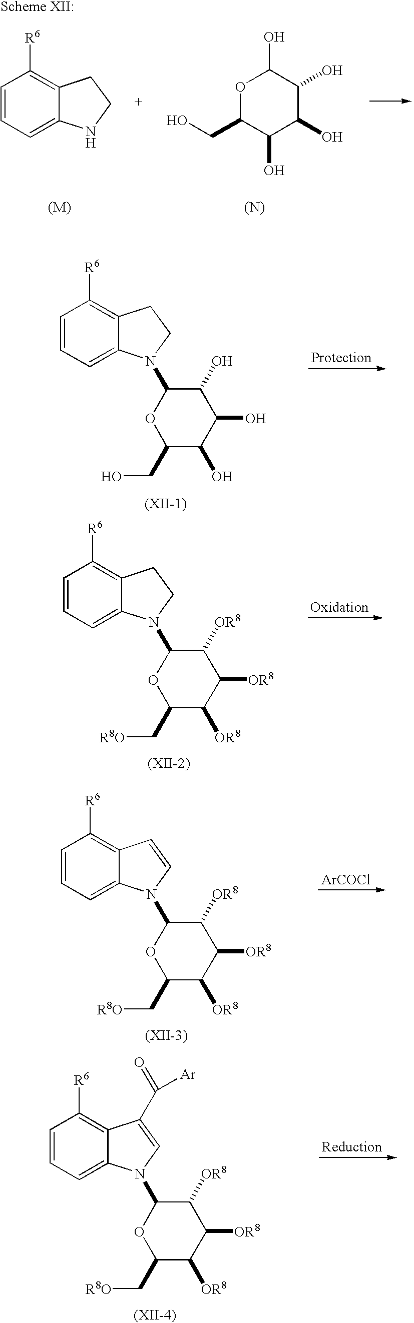

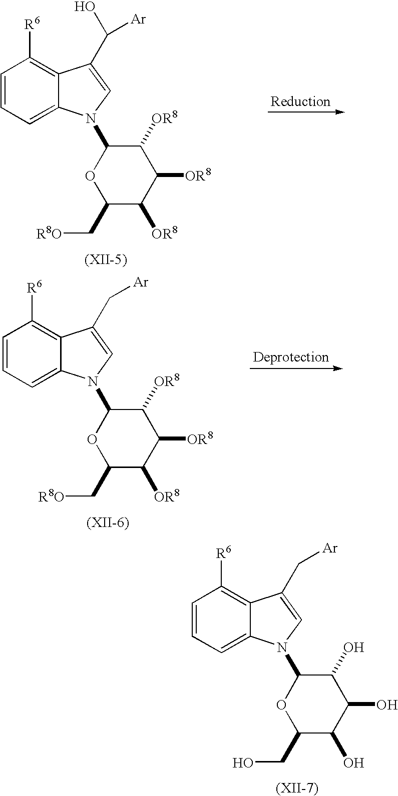

### (In the above scheme, the symbols are the same as defined above.)

### Step XII-1:

The compounds of formula (XII-1) can be prepared by condensing the compounds of formula (M) with the compounds of formula (N), or D-galactose. The condensation reaction can be carried out in accordance with Step IX-1.

### Step XII-2:

The compounds of formula (XII-2) can be prepared by protecting hydroxy groups of the compounds of formula (XII-1). The protection can be carried out in accordance with Step I-2.

### Step XII-3:

The compounds of formula (XII-3) can be prepared by oxidation of the compounds of formula (XII-2). The oxidation reaction can be carried out in accordance with Step IX-3.

### Step XII-4:

The compounds of formula (XII-4) can be prepared by condensing the compounds of formula (XII-3) with Ar—COCl, wherein Ar is the same as defined above. The condensation can be carried out in accordance with Step IX-4.

### Step XII-5:

The compounds of formula (XII-5) can be prepared by reducing the compounds of formula (XII-4). The reduction can be carried out in accordance with Step IX-5.

### Step XII-6:

The compounds of formula (XII-6) can be prepared by reducing the compounds of formula (XII-5). The reduction can be carried out in accordance with Step IX-6.

### Step XII-7:

The compounds of formula (XII-7) or a pharmaceutically acceptable salt thereof, can be prepared by deprotecting compounds of formula (XII-6), followed by converting the resulting compound into a pharmaceutically acceptable salt, if desired. This step can be carried out in accordance with Step I-6.

The compounds of formula (J) can be prepared in accordance with the following scheme:

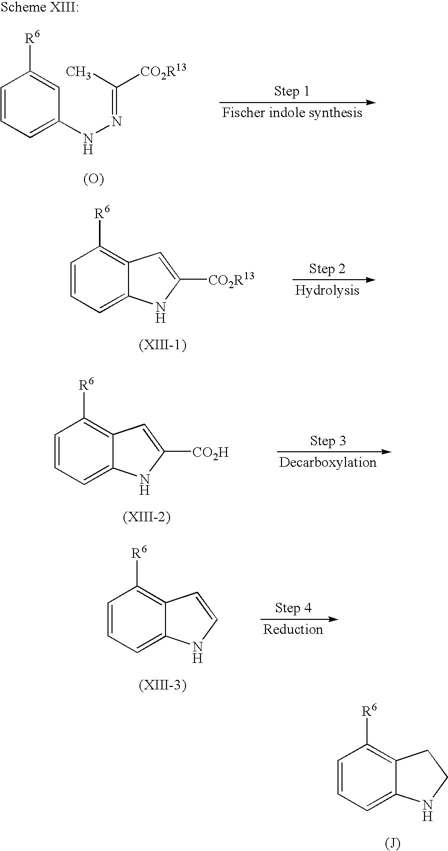

### (In the above scheme, R¹³ is alkyl, and the other symbols are the same as defined above.)

### Step XIII-1:

The compounds of formula (XIII-1) can be prepared by cyclizing the compounds of formula (O). The cyclization reaction can be carried out according to Fischer indole synthesis well known in the art (cf.: Chem. Rev., 63, 373, 1963). This reaction is typically carried out in a suitable solvent such as alcohols (e.g., methanol and ethyl alcohol) and hydrocarbons (e.g., toluene, nitrobenzene) or without solvent with an acid such as Lewis acid (e.g., zinc chloride), inorganic acid (e.g., hydrochloric acid and polyphosphoric acid) and organic acid (e.g., acetic acid and trifluoroacetic acid) at elevated temperature.

### Step XIII-2:

The compounds of formula (XIII-2) can be prepared by hydrolyzing the compounds of formula (XIII-1). The hydrolysis reaction can be typically carried out in s suitable solvent such as water, alcohols (e.g., methanol and ethyl alcohol) and ethers (e.g., dioxane and tetrahydrofuran) with a base such as alkalimetal hydroxides (e.g., lithium hydroxide, potassium hydroxide and sodium hydroxide) at lowered, ambient or elevated temperature.

### Step XIII-3:

The compounds of formula (XIII-3) can be prepared by decarboxylation of the compounds of formula (XIII-2). The decarboxylation can be typically carried out in a suitable solvent such as quinoline with a catalyst such as copper at elevated temperature.

### Step XIII-4:

The compounds of formula (J) can be prepared by reducing the compounds of formula (XIII-3). The reduction reaction can be typically carried out in a suitable solvent such as acetonitrile, halogenoalkanes (e.g., dichloromethane and dichloroethane) and ethers (e.g., diethyl ether and tetrahydrofuran) with a reducing agent such as triethylsilane, zinc borohydride in the presence of an acid including a Lewis acid such as trifluoroacetic acid, boron trifluoride.diethyl ether complex at ambient or elevated temperature.

The compounds of formula (O) can be prepared by condensing the compounds of formula (P):

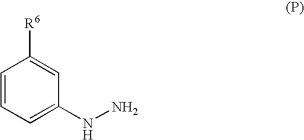

wherein the symbols are the same as defined above, with CH₃COC₂R¹³ wherein R¹³ is as defined above. The condensation reaction can be typically carried out in a suitable solvent such as acetonitrile, water and alcohols (e.g., methanol, ethyl alcohol and 1-propanol) with or without a base (e.g., sodium acetate and potassium acetate) or an acid (e.g., hydrochloric acid and acetic acid) at ambient or elevated temperature.

Alternatively, the compounds of formula (O) can be prepared by (1) reacting the compounds of formula (Q):

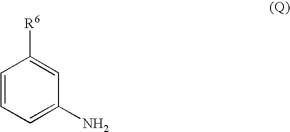

wherein the symbols are as defined above, with sodium nitrite in the presence of an acid such as hydrochloric acid in a suitable solvent such as water and alcohols (e.g., methanol and ethyl alcohol) at ambient or lowered temperature, to give a corresponding aryldiazonium salt, and (2) condensing the aryldiazonium salt with CH₃COCH(CH₃)CO₂R¹³ wherein R¹³ is as defined above, in the presence of a base such as sodium acetate, potassium hydroxide in a suitable solvent such as water and alcohols (e.g., methanol and ethyl alcohol) at lowered or ambient temperature.

The compounds of formula (G) can be prepared in accordance with the following scheme:

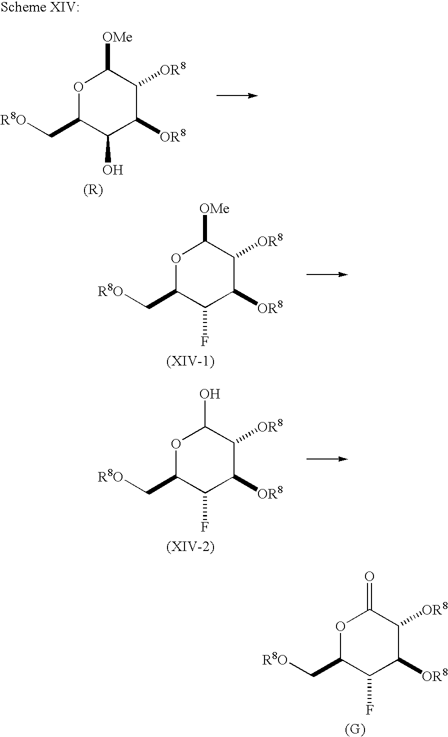

In the above scheme, Me is methyl and the other symbols are the same as defined above.

The compounds of formula (XIV-1) can be prepared by fluorination of the compounds of formula (R). The fluorination can be carried out in accordance with Step I-5.

The compounds of formula (XIV-2) can be prepared by dealkylation of the compounds of formula (XIV-1). The dealkylation is typically carried out in a mixture of sulfuric acid and acetic acid at elevated temperature.

The compounds of formula (G) can be prepared by oxidation of the compounds of formula (XIV-2). The oxidation is typically carried out in the presence of oxidizing agent (e.g., acetic anhydride-DMSO) in a suitable solvent (e.g., dichloromethane), or without a solvent at lowered or ambient temperature.

The compounds of formula (R) may be easily prepared by conventional methods well known to those skilled in the art (for example, see Tetrahedron Lett. 2000, 41, 5547-5551).

The other starting compounds are commercially available or may be easily prepared by conventional methods well known to those skilled in the art (for example, see: J. Chem. Soc. Perkin Trans. 1 1990, 2763-27692 ; Tetrahedron 1994, 50, 4215-4224; J. Chem. Soc. 1964, 3242-3254).

Hereinafter, the present invention will be illustrated by Examples and Reference Examples, but the present invention should not be construed to be limited thereto.

### EXAMPLES

### Example 1

### 3-(4-Ethylphenylmethyl)-1-(5-thio-β-D-glucopyranosyl)indole

(1) Penta-O-acetyl-5-thio-D-glucopyranose (813 mg) was suspended in ethyl alcohol (20 ml), and thereto was added sodium methoxide (28% methanol solution, 2 drops). The mixture was stirred at room temperature for one hour under argon atmosphere to give a solution of 5-thio-D-glucopyranose. To the solution was added indoline (238 mg), and the resultant mixture was refluxed overnight. Thereto was added acetic acid (2 drops), and the mixture was again refluxed for 7 hours. After being cooled to room temperature, the solvent was evaporated under reduced pressure to give crude 1-(5-thio-β-D-glucopyranosyl)indoline, which was used in the subsequent step without further purification.

(2) The above compound was dissolved in chloroform (20 ml), and thereto were added successively acetic anhydride (1.51 ml), pyridine (1.29 ml) and 4-(dimethylamino)pyridine (24 mg). After being stirred at room temperature for 2.5 days, the organic solvent was evaporated under reduced pressure. The residue was dissolved in ethyl acetate, and the mixture was successively washed with a 1 N aqueous hydrochloric acid solution, water and a saturated aqueous sodium hydrogen carbonate solution. After being dried over magnesium sulfate and treated with activated carbon, the insoluble materials were filtered off, and the filtrate was evaporated under reduced pressure. The residue was purified by silica gel column chromatography (hexane:ethyl acetate=4:1) and recrystallized from methanol to give 1-(2,3,4,6-tetra-O-acetyl-5-thio-β-D-glucopyranosyl)indoline (321 mg) as colorless needles. mp 163-165° C. APCI-Mass m/Z 466 (M+H).

(3) The above compound (310 mg) was dissolved in 1,4-dioxane (10 ml), and thereto were added 2,3-dichloro-5,6-dicyano-1,4-benzoquinone (159 mg) and H₂O (3 drops). After being stirred at room temperature for 2 hours, thereto was added a saturated aqueous sodium hydrogen carbonate solution (10 ml), and the organic solvent was evaporated under reduced pressure. The residue was extracted with ethyl acetate, and the organic layer was washed with brine. After being dried over magnesium sulfate, the insoluble materials were filtered off, and the filtrate was evaporated under reduced pressure. The residue was purified by silica gel column chromatography (hexane:ethyl acetate=2:1) to give 1-(2,3,4,6-tetra-O-acetyl-5-thio-β-D-glucopyranosyl)indole (308 mg) as a colorless solid. APCI-Mass m/Z 481 (M+NH₄).

(4) To a stirred solution of the above compound (305 mg) and 4-ethylbenzoyl chloride (166 mg) in dichloromethane (15 ml) was added aluminum chloride (439 mg) at 0° C. After being stirred at 0° C. for 30 minutes and then at room temperature for 5 hours, the mixture was poured into ice-water. Thereto was added a 1 N aqueous hydrochloric acid solution (10 ml), and the resultant mixture was extracted with chloroform twice. The combined organic layer was washed with a saturated aqueous sodium hydrogen carbonate solution and dried over magnesium sulfate. The insoluble materials were filtered off, and the filtrate was evaporated under reduced pressure. The residue was purified by silica gel column chromatography (hexane:ethyl acetate=3:1-2:1) to give 4-ethylphenyl 1-(2,3,4,6-tetra-O-acetyl-5-thio-β-D-glucopyranosyl)indol-3-yl ketone (354 mg) as a colorless powder. APCI-Mass m/z 596 (M+H).

(5) To a stirred solution of the above compound (347 mg) in ethyl alcohol (5 ml)-tetrahydrofuran (10 ml) were added cerium(III) chloride heptahydrate (648 mg) and sodium borohydride (66 mg) at 0° C. After being stirred at same temperature for 2 hours, thereto was added a 0.5 N aqueous hydrochloric acid solution (8.5 ml), and the mixture was extracted with ethyl acetate (25 ml) twice. The combined organic layer was washed with a saturated aqueous sodium hydrogen carbonate solution and dried over magnesium sulfate. The insoluble materials were filtered off, and the filtrate was evaporated under reduced pressure to give crude 4-ethylphenyl 1-(2,3,4,6-tetra-O-acetyl-5-thio-β-D-glucopyranosyl)indol-3-yl methanol (387 mg), which was used in the subsequent step without further purification.

(6) The above compound was dissolved in acetonitrile (10 ml)-dichloromethane (5 ml), and thereto were added triethylsilane (0.46 ml) and boron trifluoride-diethyl ether complex (0.37 ml) at −10° C. under argon atmosphere. After being stirred at same temperature for one hour, thereto was added a saturated aqueous sodium hydrogen carbonate solution (25 ml), and the organic solvent was evaporated under reduced pressure. The residue was extracted with ethyl acetate (25 ml) twice, and the combined organic layer was dried over magnesium sulfate. The insoluble materials were filtered off, and the filtrate was evaporated under reduced pressure. The residue was purified by silica gel column chromatography (hexane ethyl acetate=4:1) to give 3-(4-ethylphenylmethyl)-1-(2,3,4,6-tetra-O-acetyl-5-thio-β-D-glucopyranosyl)indole (290 mg) as a colorless solid. APCI-Mass m/z 599 (M+NH₄).

(7) The above compound (281 mg) was dissolved in methanol (5 ml)-tetrahydrofuran (5 ml), and thereto was added sodium methoxide (28% methanol solution, 2 drops). After being stirred at room temperature for 2 hours under argon atmosphere, the reaction solvent was evaporated under reduced pressure. The residue was purified by silica gel column chromatography (chloroform:methanol=19:1) to give the titled compound, 3-(4-ethylphenylmethyl)-1-(5-thio-β-D-glucopyranosyl)indole (190 mg) as a colorless powder. APCI-Mass m/z 414 (M+H). ¹H-NMR (DMSO-d6) δ 1.14 (t, J=7.5 Hz, 3H), 2.54 (q, J=7.5 Hz, 2H), 3.07 (m, 1H), 3.20-3.45 (m, 2H), 3.57 (m, 1H), 3.79 (m, 1H), 3.91 (m, 1H), 3.96 (s, 2H), 4.69 (t, J=5.5 Hz, 1H), 5.06 (d, J=4.8 Hz, 1H), 5.09 (d, J=4.5 Hz, 1H), 5.15 (d, J=5.5 Hz, 1H), 5.51 (d, J=10.1 Hz, 1H), 6.98 (t, J=7.4 Hz, 1H), 7.10 (d, J=7.9 Hz, 2H), 7.11 (t, J=7.2 Hz, 1H), 7.17 (s, 1H), 7.20 (d, J=8.0 Hz, 2H), 7.42 (d, J=7.9 Hz, 1H), 7.57 (d, J=8.3 Hz, 1H).

### Example 2

### 4-Chloro-3-(4-ethylphenylmethyl)-1-(5-thio-β-D-glucopyranosyl)indole

(1) Penta-O-acetyl-5-thio-D-glucopyranose (1323 mg) was suspended in ethyl alcohol (30 ml), and thereto was added sodium methoxide (28% methanol solution, 2 drops). The mixture was stirred at room temperature for one hour under argon atmosphere to give a solution of 5-thio-D-glucopyranose. To the solution were added 4-chloroindoline (500 mg) and ammonium chloride (174 mg), and the resultant mixture was refluxed for 22 hours. After being cooled to room temperature, the solvent was evaporated under reduced pressure to give crude 4-chloro-1-(5-thio-β-D-glucopyranosyl)indoline, which was used in the subsequent step without further purification.

(2) The above compound was dissolved in chloroform (20 ml), and thereto were added successively acetic anhydride (2.45 ml), pyridine (2.10 ml) and 4-(dimethylamino)pyridine (40 mg). After being stirred at room temperature overnight, the organic solvent was evaporated under reduced pressure. The residue was dissolved in ethyl acetate, and the mixture was successively washed with a 10% aqueous citric acid solution, water and a saturated aqueous sodium hydrogen carbonate solution. After being dried over magnesium sulfate and treated with activated carbon, the insoluble materials were filtered off, and the filtrate was evaporated under reduced pressure. The residue was purified by silica gel column chromatography (hexane:ethyl acetate=3:1) to give 4-chloro-1-(2,3,4,6-tetra-O-acetyl-5-thio-β-D-glucopyranosyl)indoline (1157 mg) as a pale yellow solid. APCI-Mass m/z 500/502 (M+H).

(3) The above compound was treated in a manner similar to Example 1-(3) to give 4-chloro-1-(2,3,4,6-tetra-O-acetyl-5-thio-β-D-glucopyranosyl)indole as a colorless solid. APCI-Mass m/z 515/517 (M+NH₄).

(4) The above compound and 4-ethylbenzoyl chloride were treated in a manner similar to Example 1-(4), (5), (6) and (7) to give the titled compound, 4-chloro-3-(4-ethylphenylmethyl)-1-(5-thio-β-D-glucopyranosyl)indole as a colorless powder. APCI-Mass m/z 448/450 (M+H). ¹H-NMR (DMSO-d6) δ 1.15 (t, J=7.5 Hz, 3H), 2.56 (q, J=7.7 Hz, 2H), 3.09 (m, 1H), 3.23 (t, J=8.0 Hz, 1H), 3.41 (m, 1H), 3.57 (m, 1H), 3.79 (d, J=10.0 Hz, 1H), 3.85 (m, 1H), 4.20 (d, J=15.9 Hz, 1H), 4.23 (d, J=15.9 Hz, 1H), 4.71 (m, 1H), 5.05-5.15 (m, 2H), 5.21 (d, J=5.3 Hz, 1H), 5.55 (d, J=10.1 Hz, 1H), 7.01 (d, L=7.5 Hz, 1H), 7.08-7.15 (m, 5H), 7.17 (s, 1H), 7.61 (d, J=8.3 Hz, 1H).

### Example 3

### 4-Chloro-3-(4-ethylphenylmethyl)-1-(4-fluoro-4-deoxy-β-D-glucopyranosyl)indole

(1) A suspension of 4-chloroindoline (1.00 g) and D-galactose (1.94 g) in H₂O (3.0 ml)-ethyl alcohol (20 ml) was refluxed for 29 hours under argon atmosphere. The solvent was evaporated under reduced pressure to give crude 4-chloro-1-(β-D-galactopyranosyl)indoline, which was used in the subsequent step without further purification.

(2) The above compound was suspended in chloroform (20 ml), and thereto were added successively acetic anhydride (4.92 ml), pyridine (4.21 ml) and 4-(dimethylamino)pyridine (80 mg). After being stirred at room temperature for 1.5 hours, the organic solvent was evaporated under reduced pressure. The residue was dissolved in ethyl acetate (70 ml), and the mixture was successively washed with a 10% aqueous copper(II) sulfate solution and a saturated aqueous sodium hydrogen carbonate solution (20 ml). After being dried over magnesium sulfate, the insoluble materials were filtered off, and the filtrate was evaporated under reduced pressure. The residue was purified by silica gel column chromatography (hexane:ethyl acetate=90:10-60:40) to give 4-chloro-1-(2,3,4,6-tetra-O-acetyl-β-D-galactopyranosyl)indoline (2.34 g) as a pale yellow solid. APCI-Mass m/z 484/486 (M+H).

(3) The above compound was treated in a manner similar to Example 1-(3) to give 4-chloro-1-(2,3,4,6-tetra-O-acetyl-β-D-galactopyranosyl)indole as a colorless solid. APCI-Mass m/z 499/501 (M+NH₄).

(4) The above compound and 4-ethylbenzoyl chloride were treated in a manner similar to Example 1-(4), (5), (6) and (7) to give 4-chloro-3-(4-ethylphenylmethyl)-1-(β-D-galactopyranosyl)indole as a colorless powder. APCI-Mass m/z 432/434 (M+H). ¹H-NMR (DMSO-d6) δ 1.15 (t, J=7.5 Hz, 3H), 2.56 (q, J=7.5 Hz, 2H), 3.43-3.55 (m, 3H), 3.67 (t, J=6.0 Hz, 1H), 3.77 (d, J=2.4 Hz, 1H), 3.98 (t, J=9.0 Hz, 1H), 4.23 (s, 2H), 4.64 (t, J=5.5 Hz, 1H), 4.68 (d, J=4.7 Hz, 1H), 4.93 (d, J=5.6 Hz, 1H), 5.04 (d, J=5.8 Hz, 1H), 5.29 (d, J=9.0 Hz, 1H), 7.02 (d, J=7.5 Hz, 1H), 7.06-7.15 (m, 5H), 7.17 (s, 1H), 7.58 (d, J=8.2 Hz, 1H).

(5) The above compound (6.80 g) was suspended in chloroform (300 ml), and thereto were added benzaldehyde dimethyl acetal (3.54 ml) and p-toluenesulfonic acid monohydrate (0.15 g). After being stirred at room temperature for 40 minutes, the solvent was evaporated under reduced pressure, and the residue was dissolved in ethyl acetate (200 ml). The mixture was washed with a saturated aqueous sodium hydrogen carbonate solution (10 ml) and dried over magnesium sulfate. The insoluble materials were filtered off, and the filtrate was evaporated under reduced pressure. The residue was purified by silica gel column chromatography (chloroform:methanol=100:0-90:10) to give 4-chloro-3-(4-ethylphenylmethyl)-1-(4,6-O-benzylidene-β-D-galactopyranosyl)indole (7.12 g) as a pale yellow powder. APCI-Mass m/z 520/522 (M+H).

(6) The above compound (7.12 g) was suspended in chloroform (300 ml), and thereto were added pyridine (6.64 ml) and benzoyl chloride (4.77 ml) at 0° C. The mixture was allowed to warm to room temperature, and stirred for 3 days. The reaction mixture was diluted with ethyl acetate (200 ml), and washed successively with a 10% aqueous hydrochloric acid solution (360 ml), a saturated aqueous sodium hydrogen carbonate solution (20 ml) twice and brine (50 ml). After being dried over magnesium sulfate, the insoluble materials were filtered off, and the filtrate was evaporated under reduced pressure to give crude 4-chloro-3-(4-ethylphenylmethyl)-1-(2,3-di-O-benzoyl-4,6-O-benzylidene-β-D-galactopyranosyl)indole, which was used in the subsequent step without further purification.

(7) A solution of the above compound in acetic acid (240 ml)-H₂O (30 ml) was stirred at 70° C. overnight. The solvent was evaporated under reduced pressure, and the residue was dissolved in ethyl acetate (300 ml). The mixture was washed with a saturated aqueous sodium hydrogen carbonate solution (20 ml) twice and brine (30 ml) and dried over magnesium sulfate. The insoluble materials were filtered off, and the filtrate was evaporated under reduced pressure. The residue was purified by silica gel column chromatography (chloroform:ethyl acetate=100:0-95:5) to give 4-chloro-3-(4-ethylphenylmethyl)-1-(2,3-di-O-benzoyl-β-D-galactopyranosyl)indole (7.06 g) as a pale yellow solid. APCI-Mass m/z 657/659 (M+NH₄).

(8) The above compound (7.06 g) was suspended in 2,4,6-trimethylpyridine (70 ml), and thereto was added benzoyl chloride (1.54 ml) at −40° C. The mixture was allowed to warm to 0° C., and stirred overnight. The reaction mixture was adjusted to pH 5 with a 2 N aqueous hydrochloric acid solution and extracted with ethyl acetate (100 ml)-tetrahydrofuran (100 ml). The mixture was successively washed with water (50 ml) twice, a saturated aqueous sodium hydrogen carbonate solution (20 ml) twice and brine (20 ml) twice and dried over magnesium sulfate. The insoluble materials were filtered off, and the filtrate was evaporated under reduced pressure. The residue was purified by silica gel column chromatography (hexane:ethyl acetate=90:10-50:50) and recrystallized from ethyl alcohol (100 ml) to give 4-chloro-3-(4-ethylphenylmethyl)-1-(2,3,6-tri-O-benzoyl-β-D-galactopyranosyl)indole (5.17 g) as colorless crystals. mp 192-193° C. APCI-Mass m/z 761/763 (M+NH₄).

(9) The above compound (700 mg) was dissolved in dichloromethane (70 ml), and thereto was added (diethylamino)sulfur trifluoride (1.24 ml) at 0° C. under argon atmosphere. After being stirred at room temperature for 24 hours, thereto was added cold water, and the mixture was extracted with chloroform (100 ml) twice. The combined organic layer was washed with brine (30 ml) and dried over magnesium sulfate. The insoluble materials were filtered off, and the filtrate was evaporated under reduced pressure. The residue was purified by silica gel column chromatography (chloroform:ethyl acetate=100:0-95:5) and recrystallized from diisopropyl ether (20 ml) to give 4-chloro-3-(4-ethylphenylmethyl)-1-(2,3,6-tri-O-benzoyl-4-fluoro-4-deoxy-β-D-glucopyranosyl)indole (452 mg) as pale yellow crystals. mp 137-138° C. APCI-Mass m/z 746/748 (M+H).

(10) The above compound was treated in a manner similar to Example 1-(7) to give the titled compound, 4-chloro-3-(4-ethylphenylmethyl)-1-(4-fluoro-4-deoxy-β-D-glucopyranosyl)indole as a colorless powder. APCI-Mass m/z 434/436 (M+H). ¹H-NMR (DMSO-d₆) δ 1.16 (t, J=7.5 Hz, 3H), 2.55 (q, J=7.7 Hz, 2H), 3.50 (ddd, J=12.3, 6.5 and 6.3 Hz, 1H), 3.61 (dd, J=12.1, 5.5 Hz, 1H), 3.70-3.76 (m, 2H), 3.78-3.80 (m, 1H), 4.23 (d, J=3.1 Hz, 2H), 4.32 (dt, J=50.7, 9.2 Hz, 1H), 4.84 (t, J=5.7 Hz, 1H), 5.53 (d, J=5.5 Hz, 1H), 5.56 (d, J=8.5 Hz, 1H), 5.69 (d, J=5.0 Hz, 1H), 7.04 (d, J=7.4 Hz, 1H), 7.09-7.15 (m, 5H), 7.24 (s, 1H), 7.55 (d, J=8.2 Hz, 1H).

### Example 4

### 4-Chloro-3-(4-ethoxyphenylmethyl)-1-(5-thio-β-D-glucopyranosyl)indole

4-Chloro-1-(2,3,4,6-tetra-O-acetyl-5-thio-β-D-glucopyranosyl)indole obtained in Example 2-(3) and 4-ethoxybenzoyl chloride were treated in a manner similar to Example 1-(4), (5), (6) and (7) to give the titled compound as a colorless powder. APCI-Mass m/z 464/466 (M+H). ¹H-NMR (DMSO-d₆) δ 1.30 (t, J=7.0 Hz, 3H), 3.08 (m, 1H), 3.23 (m, 1H), 3.40 (td, J=9.6, 4.8 Hz, 1H), 3.54-3.59 (m, 1H), 3.77-3.88 (m, 2H), 3.97 (q, J=6.9 Hz, 2H), 4.16 and 4.21 (ABq, J=15.8 Hz, 2H), 4.71 (t, J=5.4 Hz, 1H), 5.09 (d, J=4.8 Hz, 1H), 5.11 (d, J=4.5 Hz, 1H), 5.21 (d, J=5.3 Hz, 1H), 5.55 (d, J=9.5 Hz, 1H), 6.82 (d, J=8.5 Hz, 2H), 7.01 (d, J=7.5 Hz, 1H), 7.09-7.15 (m, 2H), 7.11 (d, J=8.7 Hz, 2H), 7.60 (d, J=8.2 Hz, 1H).

### Example 5

### 3-(Benzo[b]thiophen-2-ylmethyl)-4-chloro-1-(4-fluoro-4-deoxy-β-D-galactopyranosyl)benzene

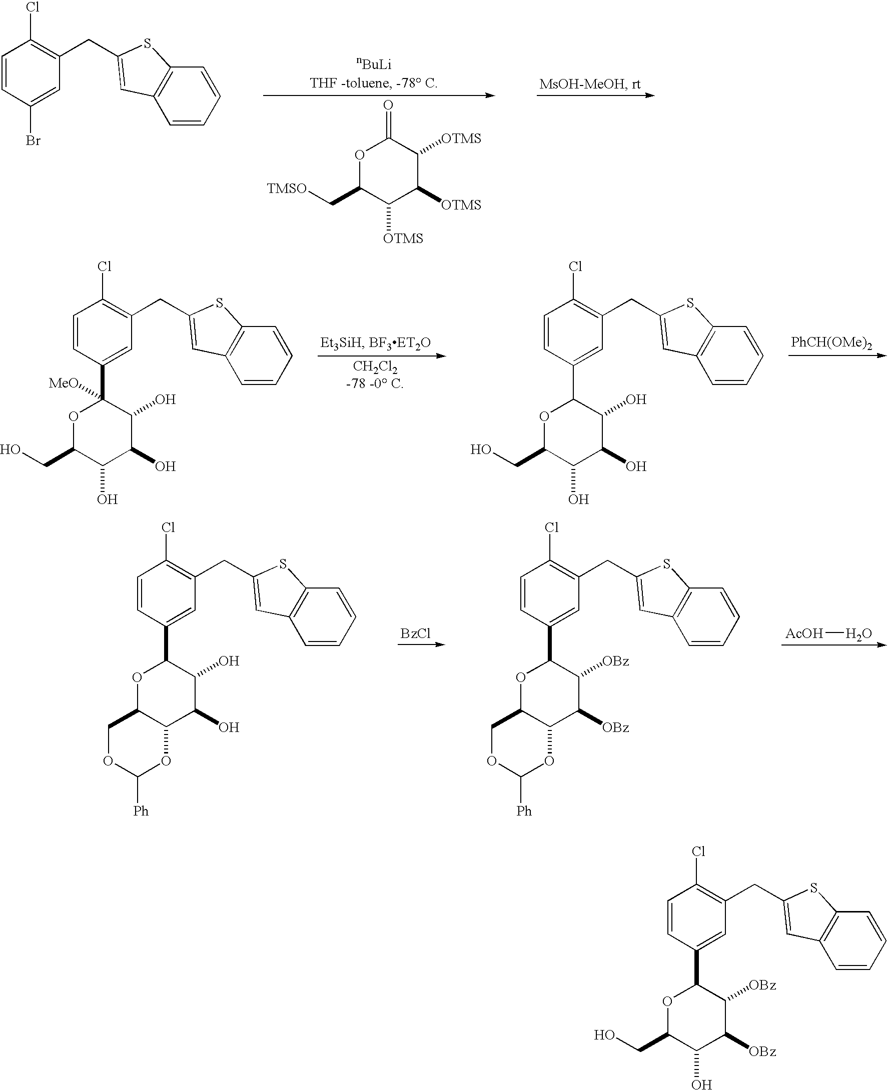

(1) 1-(Benzo[b]thiophen-2-ylmethyl)-5-bromo-2-chlorobenzene (1.00 g) was dissolved in tetrahydrofuran (8 ml)-toluene (16 ml), and the mixture was cooled to −78° C. under argon atmosphere. Thereto was added dropwise n-butyl lithium (1.59 M hexane solution, 1.86 ml) over 15 minutes, and the mixture was stirred at the same temperature for 30 minutes. A solution of 2,3,4,6-tetrakis-O-trimethylsilyl-D-glucono-1,5-lactone (see U.S. Pat. No. 6,515,117) (1.26 g) in toluene (10 ml) was added dropwise to the reaction solution over 30 minutes, and the resultant mixture was further stirred at the same temperature for 1.5 hours. Thereto was added a solution of methanesulfonic acid (0.58 ml) in methanol (20 ml), and the mixture was stirred at room temperature overnight. Under ice-cooling, to the mixture was added a saturated aqueous sodium hydrogen carbonate solution (200 ml), and the mixture was extracted with ethyl acetate. The organic layer was washed with brine and dried over sodium sulfate. The insoluble materials were filtered off, and the filtrate was evaporated under reduced pressure. The residue was purified by silica gel column chromatography (chloroform:methanol=10:0-9:1) to give 3-(benzo[b]thiophen-2-ylmethyl)-4-chloro-1-(1-α-methoxy-β-D-glucopyranosyl)benzene (875 mg) as a colorless powder.

(2) A solution of the above compound (1.91 g) and triethylsilane (2.03 ml) in dichloromethane (80 ml) was cooled to −78° C. under argon atmosphere, and thereto was added boron trifluoride-diethyl ether complex (1.61 ml). The mixture was allowed to warm to 0° C. and stirred at the same temperature for 3 hours. To the resultant mixture was added a saturated aqueous sodium hydrogen carbonate solution, and the mixture was extracted with chloroform. The organic layer was washed with brine and dried over sodium sulfate. The insoluble materials were filtered off, and the filtrate was evaporated under reduced pressure. The residue was purified by silica gel column chromatography (chloroform:methanol=10:0-9:1) and crystallization from ethyl acetate-diethyl ether to give 3-(benzo[b]thiophen-2-ylmethyl)-4-chloro-1-(β-D-glucopyranosyl)benzene (1.29 g) as colorless crystals. mp 153-154° C. APCI-Mass m/z 438/440 (M+NH₄).

(3) The above compound was treated in a manner similar to Example 3-(5), (6), (7) and (8) to give 3-(benzo[b]thiophen-2-ylmethyl)-4-chloro-1-(2,3,6-tri-O-benzoyl-β-D-glucopyranosyl)benzene as colorless crystals. mp 215-217° C. APCI-Mass m/z 750/752 (M+NH₄).

(4) A solution of the above compound (796 mg) and 4-dimethylaminopyridine (780 mg) in dichloromethane (50 ml) was cooled to 0° C. under argon atmosphere, and thereto was added (diethylamino)sulfur trifluoride (0.63 ml). The mixture was allowed to warm to room temperature and stirred at the same temperature for 19 hours. Thereto was added cold water under ice-cooling, and the mixture was extracted with chloroform. The organic layer was washed with brine, dried over magnesium sulfate and treated with activated carbon. The insoluble materials were filtered off, and the filtrate was evaporated under reduced pressure. The residue was purified by silica gel column chromatography (hexane:ethyl acetate=4:1-2:1) and crystallization from ethyl acetate-hexane to give 3-(benzo[b]thiophen-2-ylmethyl)-4-chloro-1-(2,3,6-tri-O-benzoyl-4-fluoro-4-deoxy-β-D-galactopyranosyl)benzene (305 mg) as colorless crystals. mp 204-207° C. APCI-Mass m/z 752/754 (M+NH₄).

(5) The above compound was treated in a manner similar to Example 1-(7) to give the titled compound, 3-(benzo[b]thiophen-2-ylmethyl)-4-chloro-1-(4-fluoro-4-deoxy-β-D-galactopyranosyl)benzene as a colorless powder. APCI-Mass m/z 440/442 (M+NH₄). ¹H-NMR (DMSO-d6) δ 3.40-3.70 (m, 5H), 4.09 (d, J=9.2 Hz, 1H), 4.34 (d, J=15.9 Hz, 1H), 4.37 (d, J=15.6 Hz, 1H), 4.73 (dd, J=50.5, 2.2 Hz, 1H), 4.86 (t, J=5.6 Hz, 1H), 4.97 (d, J=5.9 Hz, 1H), 5.28 (d, J=5.3 Hz, 1H), 7.14 (s, 1H), 7.24-7.34 (m, 3H), 7.42 (d, J=1.8 Hz, 1H), 7.45 (d, J=8.2 Hz, 1H), 7.73 (d, J=7.7 Hz, 1H), 7.85 (d, J=7.7 Hz, 1H).

### Example 6

### 4-Chloro-3-(5-phenyl-2-thienylmethyl)-1-(6-fluoro-6-deoxy-β-D-glucopyranosyl)benzene

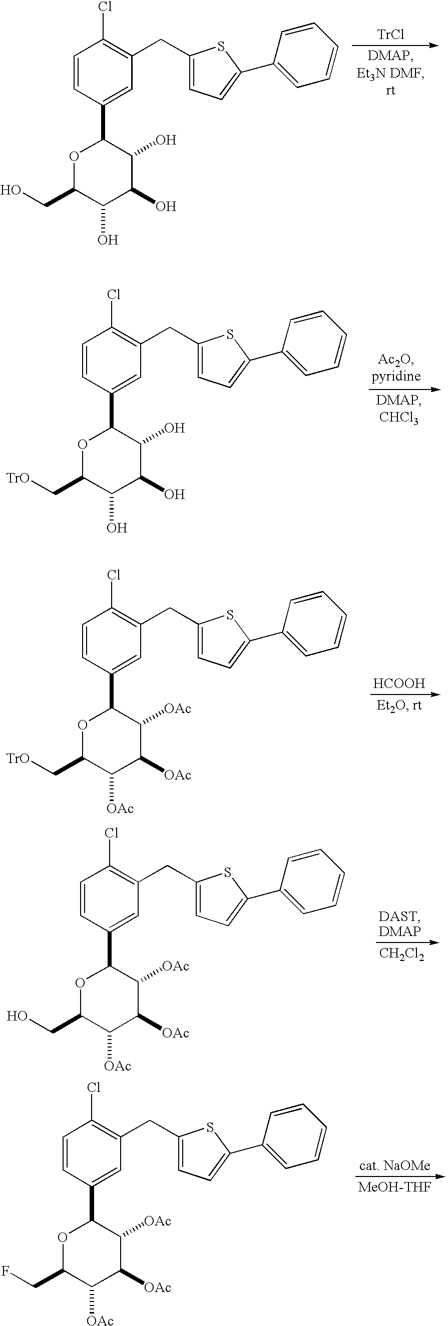

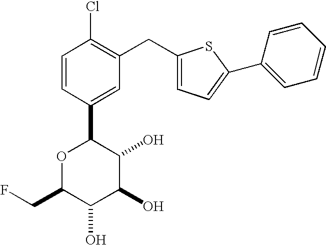

(1) 5-bromo-2-chloro-1-(5-phenyl-2-thienylmethyl)benzene was treated in a manner similar to Example 5-(1) and (2) to give 4-chloro-3-(5-phenyl-2-thienylmethyl)-1-(β-D-glucopyranosyl)benzene as a colorless powder. APCI-Mass m/z 464/466 (M+NH₄).

(2) The above compound (1.00 g) was dissolved in N,N-dimethylformamide (10 ml), and thereto were added trityl chloride (686 mg), 4-dimethylaminopyridine (14 mg) and triethylamine (0.47 ml). After being stirred at room temperature for 6 days, the mixture was diluted with ethyl acetate. The solution was successively washed with a 1 N aqueous hydrochloric acid solution, water twice, a saturated aqueous sodium hydrogen carbonate solution and brine, and the organic layer was dried over magnesium sulfate. The insoluble materials were filtered off, and the filtrate was evaporated under reduced pressure. The residue was purified by silica gel column chromatography (chloroform:methanol=100:1-19:1) to give 4-chloro-3-(5-phenyl-2-thienylmethyl)-1-(6-O-trityl-β-D-glucopyranosyl)benzene (870 mg) as a pale yellow powder. ESI-Mass m/z 711/713 (M+Na).

(3) The above compound was treated in a manner similar to Example 1-(2) to give 4-chloro-3-(5-phenyl-2-thienylmethyl)-1-(2,3,4-tri-O-acetyl-6-O-trityl-β-D-glucopyranosyl)benzene as a colorless powder. ESI-Mass m/z 837/839 (M+Na).

(4) A solution of the above compound (1.45 g) in formic acid (10 ml) and diethyl ether (5 ml) was stirred at room temperature for 3.5 hours, and then was diluted with water. The mixture was extracted with ethyl acetate, and the organic layer was successively washed with H₂O, a saturated aqueous sodium hydrogen carbonate solution and brine. After being dried over magnesium sulfate, the insoluble materials were filtered off, and the filtrate was evaporated under reduced pressure. The residue was purified by silica gel column chromatography (hexane:ethyl acetate=4:1-1:1) to give 4-chloro-3-(5-phenyl-2-thienylmethyl)-1-(2,3,4-tri-O-acetyl-β-D-glucopyranosyl)benzene (689 mg) as colorless crystals. mp 166-168° C. APCI-Mass m/z 590/592 (M+NH₄).

(5) The above compound was treated in a manner similar to Example 5-(4) and 1-(7) to give the titled compound, 4-chloro-3-(5-phenyl-2-thienylmethyl)-1-(6-fluoro-6-deoxy-β-D-glucopyranosyl)benzene as a colorless powder. APCI-Mass m/z 466/468 (M+NH₄). ¹H-NMR (DMSO-d6) δ 3.14 (m, 1H), 3.23 (m, 1H), 3.32 (m, 1H), 3.50 (m, 1H), 4.10 (d, J=9.5 Hz, 1H), 4.24 (d, J=15.4 Hz, 1H), 4.28 (d, J=15.7 Hz, 1H), 4.53 (ddd, J=47.8, 10.1 and 5.3 Hz, 1H), 4.58 (dd, J=47.8, 10.3 Hz, 1H), 4.95 (d, J=5.9 Hz, 1H), 5.10 (d, J=5.0 Hz, 1H), 5.27 (d, J=5.5 Hz, 1H), 6.88 (d, J=3.5 Hz, 1H), 7.25 (m, 2H), 7.32 (d, J=3.5 Hz, 1H), 7.35-7.45 (m, 4H), 7.56 (d, J=7.4 Hz, 2H).

### Example 7

### 4-Chloro-3-(5-phenyl-2-thienylmethyl)-1-(4-fluoro-4-deoxy-β-D-glucopyranosyl)benzene

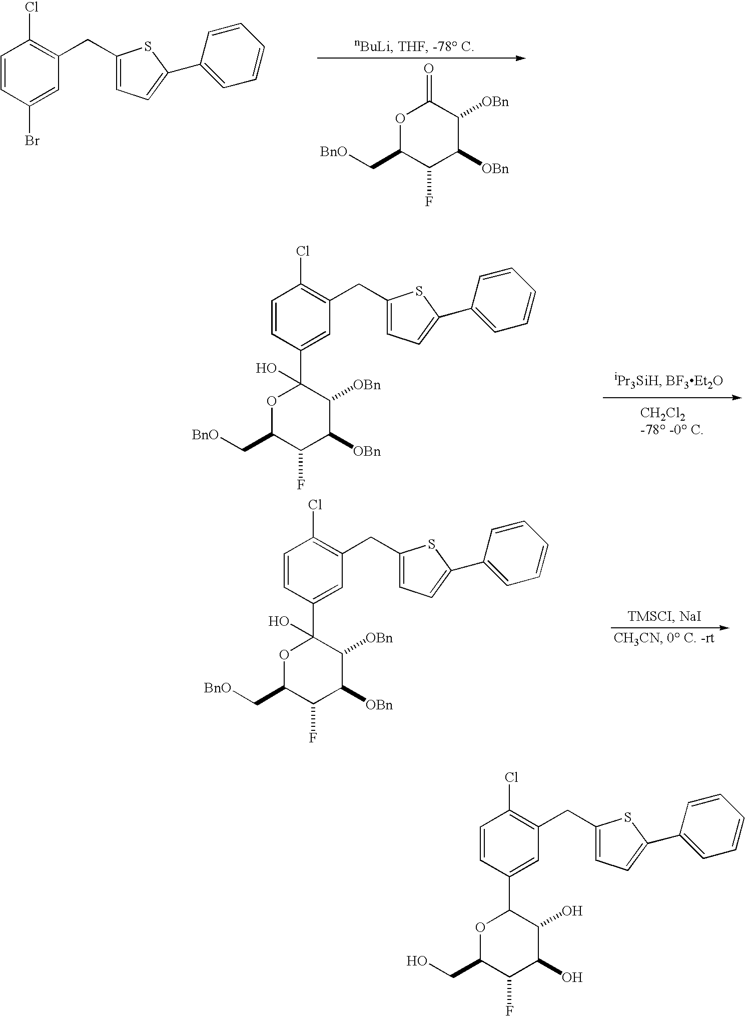

(1) 5-Bromo-2-chloro-1-(5-phenyl-2-thienylmethyl)benzene (397 mg) was dissolved in tetrahydrofuran (10 ml), and the mixture was cooled to −78° C. under argon atmosphere. Thereto was added dropwise n-butyl lithium (1.56 M hexane solution, 0.70 ml), and the mixture was stirred at the same temperature for 20 minutes. A solution of 2,3,6-tri-O-benzyl-4-fluoro-4-deoxy-D-glucono-1,5-lactone (496 mg) in tetrahydrofuran (5 ml) was added dropwise to the reaction solution over 5 minutes, and the resultant mixture was further stirred at the same temperature for 1.5 hours. Thereto was added a saturated aqueous ammonium chloride solution (5 ml), and the mixture was allowed to warm to room temperature. The mixture was diluted with H₂O and ethyl acetate, and the organic layer was separated, washed with brine and dried over magnesium sulfate. The insoluble materials were filtered off, and the filtrate was evaporated under reduced pressure to give crude 4-chloro-3-(5-phenyl-2-thienylmethyl)-1-(1-hydroxy-2,3,6-tri-O-benzyl-4-fluoro-4-deoxy-D-glucopyranosyl)benzene, which was used in the subsequent step without further purification.

(2) A solution of the above compound in dichloromethane (10 ml) was cooled to −78° C. under argon atmosphere, and thereto were added successively triisopropylsilane (0.68 ml) and boron trifluoride-diethyl ether complex (0.42 ml). After being stirred at −78° C. for 1 hour, the mixture was allowed to warm to 0° C. and stirred at the same temperature for 1 hour. To the resultant mixture was added a saturated aqueous sodium hydrogen carbonate solution, and the mixture was extracted with ethyl acetate. The organic layer was washed with brine and dried over sodium sulfate. The insoluble materials were filtered off, and the filtrate was evaporated under reduced pressure. The residue was purified by silica gel column chromatography (hexane:ethyl acetate=19:1-9:1) to give 4-chloro-3-(5-phenyl-2-thienylmethyl)-1-(2,3,6-tri-O-benzyl-4-fluoro-4-deoxy-β-D-glucopyranosyl)benzene (226 mg) as a pale yellow caramel. APCI-Mass m/z 736/738 (M+NH₄).

(3) To a solution of sodium iodide (550 mg) in acetonitrile (5 ml) was added dropwise chlorotrimethylsilane (0.47 ml) at 0° C. under argon atmosphere, and the mixture was stirred at same temperature for 20 minutes. Thereto was added dropwise a solution of the above compound (220 mg) in acetonitrile (5 ml), and the resultant mixture was stirred at 0° C. for 1 hour and at room temperature for 2.5 hours. The reaction mixture was quenched with a 10% aqueous sodium thiosulfate solution at 0° C., and the mixture was extracted with ethyl acetate. The organic layer was washed successively with H₂O, a saturated aqueous sodium hydrogen carbonate solution and brine. After being dried over magnesium sulfate, the insoluble materials were filtered off, and the filtrate was evaporated under reduced pressure. The residue was purified by silica gel column chromatography (chloroform:methanol=30:1) to give the titled compound, 4-chloro-3-(5-phenyl-2-thienylmethyl)-1-(4-fluoro-4-deoxy-β-D-glucopyranosyl)benzene (123 mg) as a colorless powder. APCI-Mass m/z 466/468 (M+NH₄). ¹H-NMR (DMSO-d6) δ 3.18 (m, 1H), 3.48-3.67 (m, 4H), 4.14 (d, J=9.5 Hz, 1H), 4.23 (d, J=16.4 Hz, 1H), 4.26 (dt, J=49.3, 9.2 Hz, 1H), 4.27 (d, J=15.9 Hz, 1H), 4.77 (t, J=5.5 Hz, 1H), 5.17 (d, J=5.9 Hz, 1H), 5.49 (d, J=5.5 Hz, 1H), 6.88 (d, J=3.7 Hz, 1H), 7.24-7.29 (m, 2H), 7.32 (d, J=3.7 Hz, 1H), 7.37 (t, J=7.7 Hz, 2H), 7.41-7.45 (m, 2H), 7.57 (d, J=7.4 Hz, 2H).

The chemical structures of the above Examples are shown in Tables 1 and 2 below:

TABLE 1

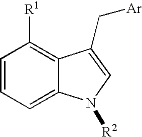

| Example | | | |
|---|---|---|---|
| No. | R¹ | Ar | R² |
| 1 | H | 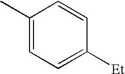 | 5-thio-β-D-glucopyranosyl |
| 2 | Cl | 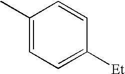 | 5-thio-β-D-glucopyranosyl |
| 3 | Cl | 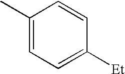 | 4-fluoro-4-deoxy-β-D-glucopyranosyl |
| 4 | Cl | 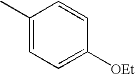 | 5-thio-β-D-glucopyranosyl |

TABLE 2

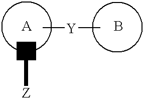

| ExampleNo. | 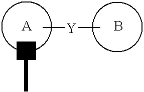 | Z |
|---|---|---|
| 5 | 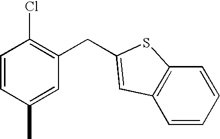 | 4-fluoro-4-deoxy-β-D-galactopyranosyl |
| 6 | 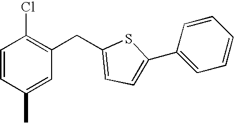 | 6-fluoro-6-deoxy-β-D-glucopyranosyl |
| 7 | 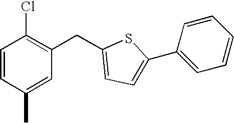 | 4-fluoro-4-deoxy-β-D-glucopyranosyl |

### Examples 8-36

The following compounds shown in Table 3 were prepared in accordance with one of the above Examples.

TABLE 3

| ExampleNo. | 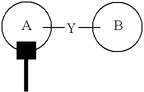 | Z | APCI-Massm/Z |
|---|---|---|---|
| 8 | 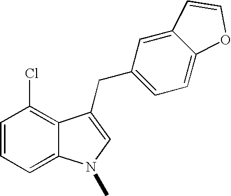 | 5-thio-β-D-glucopyranosyl | 477/479(M + NH₄) |
| 9 | 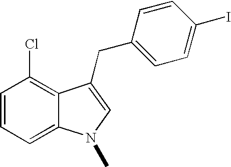 | 5-thio-β-D-glucopyranosyl | 546/548(M + H) |
| 10 | 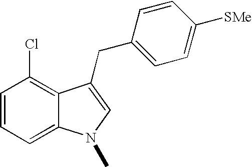 | 5-thio-β-D-glucopyranosyl | 483/485(M + NH₄) |
| 11 | 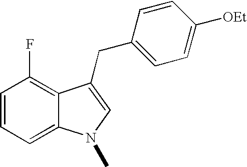 | 5-thio-β-D-glucopyranosyl | 465(M + NH₄) |
| 12 | 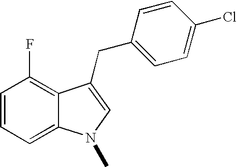 | 5-thio-β-D-glucopyranosyl | 438/440(M + H) |
| 13 | 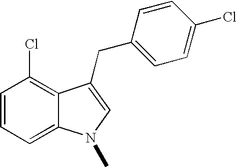 | 5-thio-β-D-glucopyranosyl | 454/456(M + H) |
| 14 | 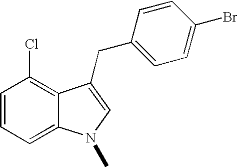 | 5-thio-β-D-glucopyranosyl | 498/500(M + H) |
| 15 | 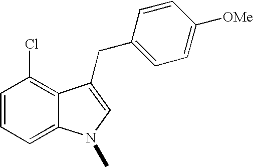 | 5-thio-β-D-glucopyranosyl | 467/469(M + NH₄) |
| 16 | 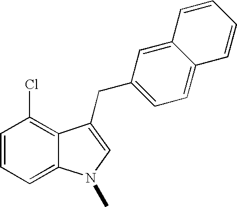 | 5-thio-β-D-glucopyranosyl | 487/489(M + NH₄) |
| 17 | 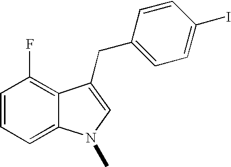 | 5-thio-β-D-glucopyranosyl | 547(M + NH₄) |
| 18 | 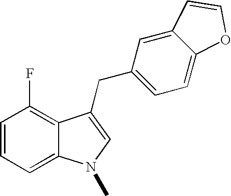 | 5-thio-β-D-glucopyranosyl | 461(M + NH₄) |
| 19 | 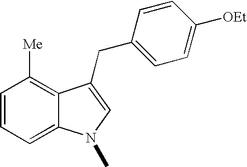 | 5-thio-β-D-glucopyranosyl | 444(M + H) |
| 20 | 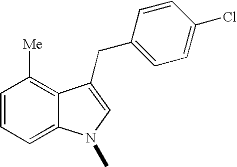 | 5-thio-β-D-glucopyranosyl | 434/436(M + H) |
| 21 | 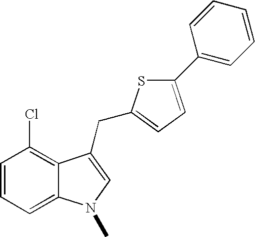 | 5-thio-β-D-glucopyranosyl | 519/521(M + NH₄) |
| 22 | 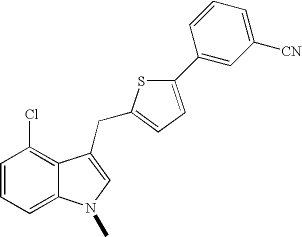 | 5-thio-β-D-glucopyranosyl | 544/546(M + NH₄) |
| 23 | 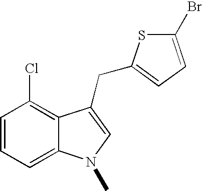 | 5-thio-β-D-glucopyranosyl | 504/506(M + H) |
| 24 | 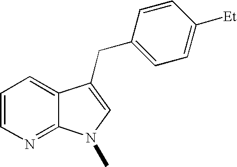 | 5-thio-β-D-glucopyranosyl | 415(M + H) |
| 25 | 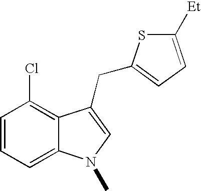 | 5-thio-β-D-glucopyranosyl | 471/473(M + NH₄) |
| 26 | 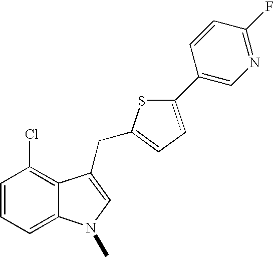 | 5-thio-β-D-glucopyranosyl | 521/523(M + H) |
| 27 | 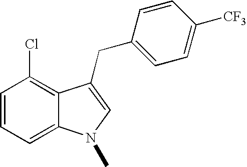 | 5-thio-β-D-glucopyranosyl | 488/490(M + H) |
| 28 | 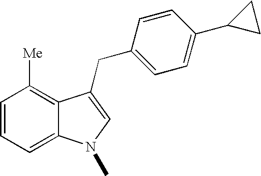 | 5-thio-β-D-glucopyranosyl | 440(M + H) |
| 29 | 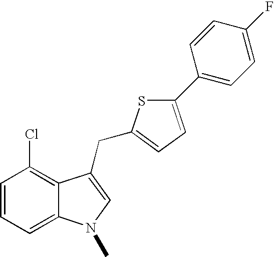 | 5-thio-β-D-glucopyranosyl | 537/539(M + NH₄) |
| 30 | 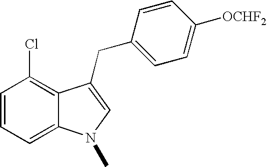 | 5-thio-β-D-glucopyranosyl | 486/488(M + H) |
| 31 | 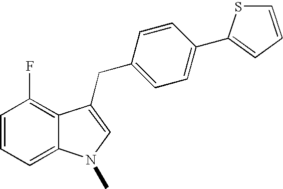 | 5-thio-β-D-glucopyranosyl | 486(M + H) |
| 32 | 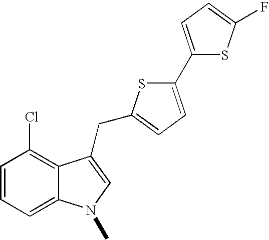 | 5-thio-β-D-glucopyranosyl | 543/545(M + NH₄) |
| 33 | 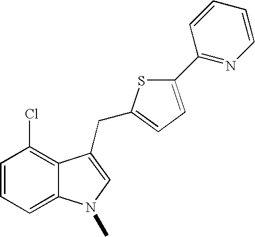 | 5-thio-β-D-glucopyranosyl | 503/505(M + H) |
| 34 | 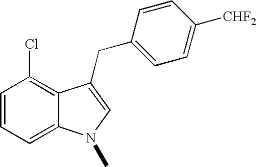 | 5-thio-β-D-glucopyranosyl | 470/472(M + H) |
| 35 | 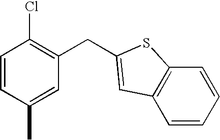 | β-D-galacto-pyranosyl | 438/440(M + NH₄) |
| 36 | 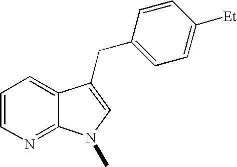 | 4-fluoro-4-deoxy-β-D-glucopyranosyl | 401(M + H) |

In the above Tables 1-3, Me is methyl, MeO is methoxy, Et is ethyl, EtO is ethoxy, and “5-thio-β-D-glucopyranosyl”, “4-fluoro-4-deoxy-β-D-glucopyranosyl”, “β-D-galactopyranosyl”, “4-fluoro-4-deoxy-β-D-galactopyranosyl”, and “6-fluoro-6-deoxy-β-D-glucopyranosyl” represent the following chemical formula, respectively:

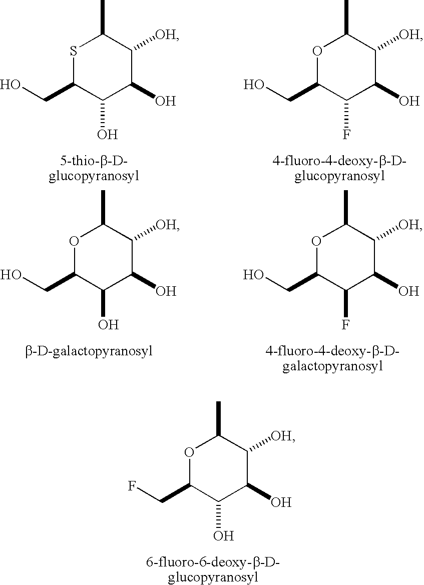

### Reference Example 1

### 4-Chloroindoline

A solution of 4-chloroindole (3.15 g) and triethylsilane (8.30 ml) in trifluoroacetic acid (32 ml) was stirred at 50° C. for 30 minutes. The solvent was evaporated under reduced pressure, and the residue was basified with a saturated aqueous sodium hydrogen carbonate solution. The mixture was extracted with ethyl acetate twice, and the combined organic layer was dried over magnesium sulfate. The insoluble materials were filtered off, and the filtrate was evaporated under reduced pressure. The residue was purified by silica gel column chromatography (hexane:ethyl acetate=100:0-80:20) to give the titled compound (2.89 g) as a colorless oil. APCI-Mass m/z 154/156 (M+H). ¹H-NMR (DMSO-d6) δ 2.94 (t, J=8.7 Hz, 2H), 3.46 (t, J=8.7 Hz, 2H), 5.83 (s, 1H), 6.40 (d, J=7.7 Hz, 1H), 6.50 (d, J=8.0 Hz, 1H), 6.90 (t, J=7.9 Hz, 1H).

### Reference Example 2

### 1-(Benzo[b]thiophen-2-ylmethyl)-5-bromo-2-chlorobenzene

(1) 5-Bromo-2-chlorobenzoic acid (10.0 g) was suspended in dichloromethane (80 ml), and thereto were added successively N,O-dimethylhydroxylamine hydrochloride (4.56 g), 1-[3-(dimethylamino)propyl]-3-ethylcarbodiimide hydrochloride (8.96 g), 1-hydroxybenzotriazole (6.31 g) and triethylamine (8.88 ml). After being stirred at room temperature for 64 hours, thereto was added a 10% aqueous hydrochloric acid solution at 0° C., and the mixture was extracted with chloroform. The organic layer was washed with a saturated aqueous sodium hydrogen carbonate solution and brine, and dried over sodium sulfate. The insoluble materials were filtered off, and the filtrate was evaporated under reduced pressure to give crude 5-bromo-2-chloro-N-methoxy-N-methylbenzamide, which was used in the subsequent step without further purification.

(2) A solution of the above compound in tetrahydrofuran (200 ml) was cooled to −78° C. under argon atmosphere, and thereto was added dropwise diisobutylaluminum hydride (1.0 M toluene solution, 64 ml). The mixture was stirred at −78° C. for 30 minutes and at 0° C. for 40 minutes. To the resultant mixture was added a 10% aqueous hydrochloric acid solution at 0°, and the mixture was extracted with ethyl acetate. The organic layer was washed with a saturated aqueous sodium hydrogen carbonate solution and brine, and dried over sodium sulfate. The insoluble materials were filtered off, and the filtrate was evaporated under reduced pressure. The residue was purified by silica gel column chromatography (hexane:ethyl acetate=19:1) to give 5-bromo-2-chlorobenzaldehyde (8.90 g) as a colorless solid.

(3) To a solution of thianaphthene (5.18 g) in tetrahydrofuran (60 ml) was added dropwise n-butyl lithium (2.44 M hexane solution, 15.8 ml) at −78° C. under argon atmosphere, and the mixture was stirred at −78° C. for 30 minutes and at 0° C. for 30 minutes. The mixture was cooled to −78° C., and thereto was added a solution of the above compound (8.90 g) in tetrahydrofuran (60 ml) in one portion. After being stirred at the same temperature for 1.5 hours, thereto was added a saturated aqueous ammonium chloride solution, and the mixture was extracted with ethyl acetate. The organic layer was washed with brine and dried over sodium sulfate. The insoluble materials were filtered off, and the filtrate was evaporated under reduced pressure. The residue was purified by silica gel column chromatography (hexane:ethyl acetate=15:1) to give benzo[b]thiophen-2-yl-(5-bromo-2-chlorophenyl)methanol (12.25 g) as a orange oil.

(4) A solution of the above compound (12.25 g) in dichloromethane (300 ml) was cooled to 0° C., and thereto were added successively triethylsilane (12.17 ml) and boron trifluoride-diethyl ether complex (6.58 ml). After being stirred at the same temperature for 2 hours, thereto was added a saturated aqueous sodium hydrogen carbonate solution, and the mixture was extracted with chloroform. The organic layer was washed with brine and dried over sodium sulfate. The insoluble materials were filtered off, and the filtrate was evaporated under reduced pressure. The residue was purified by silica gel column chromatography (hexane only) to give the titled compound, 1-(benzo[b]thiophen-2-ylmethyl)-5-bromo-2-chlorobenzene (9.30 g) as a colorless solid. ¹H-NMR (CDCl₃) δ 4.30 (s, 2H), 7.02 (d, J=0.9 Hz, 1H), 7.23-7.36 (m, 4H), 7.43 (m, 1H), 7.65-7.69 (m, 1H), 7.73-7.77 (m, 1H).

### Reference Example 3

### 5-Bromo-2-chloro-1-(5-phenyl-2-thienylmethyl)benzene

(1) 5-Bromo-2-chlorobenzoic acid (55.95 g) was suspended in dichloromethane (500 ml), and thereto were added oxalyl chloride (25 ml) and N,N-dimethylformamide (5 drops). After being stirred at room temperature overnight, thereto was added tetrahydrofuran (50 ml), and the mixture was further stirred at the same temperature for 1 hour. The solvent and volatile substance were evaporated under reduced pressure to give 5-bromo-2-chlorobenzoyl chloride. This compound and 2-phenylthiophene (34.63 g) were dissolved in dichloromethane (1000 ml), and thereto was added portionwise aluminum chloride (31.70 g) at 0° C. The mixture was stirred at 0° C. for 30 minutes and at room temperature for 18 hours. The reaction mixture was poured into an ice-water (1500 ml), and the mixture was extracted with chloroform (1000 ml). The organic layer was washed with H₂O (1000 ml) twice and a saturated aqueous sodium hydrogen carbonate solution (1000 ml) twice. After being dried over magnesium sulfate and treated with activated carbon, the insoluble materials were filtered off, and the filtrate was evaporated under reduced pressure. The residual solid was triturated with hexane (1000 ml) and collected by filtration to give 4-bromo-2-chlorophenyl 5-phenyl-2-thienyl ketone (50.22 g) as pale green crystals. mp 132-134° C. APCI-Mass m/z 377/379 (M+H).

(2) To a suspension of the above compound (50.00 g) and triethylsilane (63.44 ml) in dichloromethane (300 ml)-acetonitrile (300 ml) was added dropwise boron trifluoride-diethyl ether complex (50.18 ml) at 0° C. After being stirred at room temperature for 3 hours, thereto was added a saturated aqueous sodium hydrogen carbonate solution (800 ml), and the mixture was extracted with chloroform (300 ml) twice. The combined organic layer was washed with brine (500 ml), dried over magnesium sulfate and treated with activated carbon. The insoluble materials were filtered off, and the filtrate was evaporated under reduced pressure. The residue was dissolved in ethyl acetate (300 ml), and the solution was treated with activated carbon. The insoluble materials were filtered off, and the filtrate was evaporated under reduced pressure. The residue was crystallized from methanol (150 ml) to give the titled compound,

5-bromo-2-chloro-1-(5-phenyl-2-thienylmethyl)benzene (42.92 g) as pale yellow crystals. mp 80-81° C. ¹H-NMR (CDCl₃) δ 4.22 (s, 2H), 6.79 (dt, J=3.5, 1.0 Hz, 1H), 7.14 (d, J=3.7 Hz, 1H), 7.21-7.38 (m, 5H), 7.41 (dd, J=2.4, 0.4 Hz, 1H), 7.52-7.57 (m, 2H).

### Reference Example 4

### 2,3,6-Tri-O-benzyl-4-fluoro-4-deoxy-D-glucono-1,5-lactone

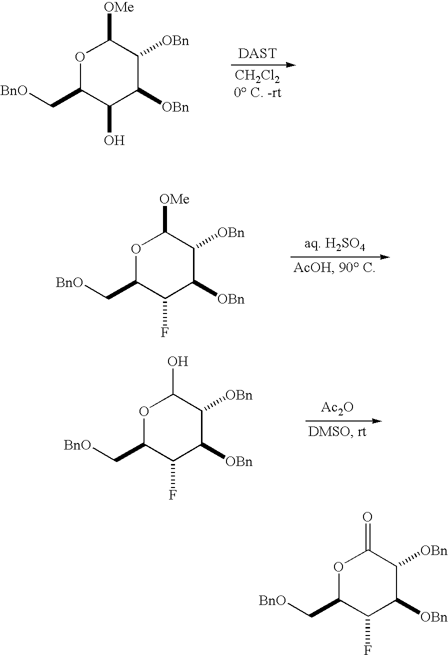

(1) Methyl 2,3,6-tri-O-benzyl-β-D-galactopyranoside (see Sakagami, M.; Hamana, H. Tetrahedron Lett. 2000, 41, 5547-5551.), (16.99 g) was dissolved in dichloromethane (172 ml), and thereto was added (diethylamino)sulfur trifluoride (9.71 ml) at 0° C. The mixture was stirred at 0° C. for 2 hours and at room temperature for 5 hours. The resultant mixture was cooled to 0° C., and thereto was added a saturated aqueous sodium hydrogen carbonate solution. The organic layer was separated and dried over sodium sulfate. The insoluble materials were filtered off, and the filtrate was evaporated under reduced pressure. The residue was purified by silica gel column chromatography (hexane:ethyl acetate=100:0-90:10) to give methyl 2,3,6-tri-O-benzyl-4-fluoro-4-deoxy-β-D-glucopyranoside (5.86 g) as a pale yellow oil. APCI-Mass m/z 484 (M+NH₄).

(2) A mixture of the above compound (5.39 g) in a 3 M aqueous sulfuric acid solution (10 ml)-acetic acid (50 ml) was stirred at 90° C. for 5 hours. After being cooled to room temperature, thereto was added H₂O, and the mixture was extracted with ethyl acetate. The organic layer was washed with successively H₂O twice, a saturated aqueous sodium hydrogen carbonate solution and brine. After being dried over magnesium sulfate, the insoluble materials were filtered off, and the filtrate was evaporated under reduced pressure. The residue was purified by silica gel column chromatography (hexane:ethyl acetate=4:1) to give 2,3,6-tri-O-benzyl-4-fluoro-4-deoxy-D-glucose (2.39 g) as a pale yellow oil. APCI-Mass m/z 470 (M+NH₄).

(3) The above compound (2.76 g) was dissolved in dimethyl sulfoxide (16 ml), and thereto was added acetic anhydride (11 ml) at 0° C. under argon atmosphere. The mixture was stirred at room temperature overnight, and thereto was added an ice-water at 0° C. The resultant mixture was extracted with ethyl acetate, and the organic layer was washed with a cold water twice and dried over magnesium sulfate. The insoluble materials were filtered off, and the filtrate was evaporated under reduced pressure. The residue was purified by silica gel column chromatography (hexane:ethyl acetate=19:1-9:1) to give the titled compound, 2,3,6-tri-O-benzyl-4-fluoro-4-deoxy-D-glucono-1,5-lactone (2.08 g) as a colorless solid. APCI-Mass m/z 468 (M+NH₄). ¹H-NMR (CDCl₃) δ 3.74 (ddd, J=11.0, 3.3 and 1.7 Hz, 1H), 3.79 (ddd, J=11.2, 2.6 and 1.7 Hz, 1H), 3.99 (ddd, J=19.8, 6.8 and 6.0 Hz, 1H), 4.15 (d, J=6.8 Hz, 1H), 4.52-4.65 (m, 4H), 4.70 (s, 2H), 4.92 (ddd, J=49.6, 7.6 and 6.0 Hz, 1H), 4.93 (d, J=11.4 Hz, 1H), 7.23-7.38 (m, 15H).

### Pharmacological Experiments

### 1. Assay for SGLT2 Inhibition

### Test Compounds:

Compounds described in the above examples were used for the SGLT2 inhibition assay.

### Method:

CHOK1 cells expressing human SGLT2 were seeded in 24-well plates at a density of 400,000 cells/well in F-12 nutrient mixture (Ham's F-12) containing 10% fetal bovine serum, 400 μg/ml Geneticin, 50 units/ml sodium penicillin G (Gibco-BRL) and 50 μg/ml streptomycin sulfate. After 2 days of culture at 37° C. in a humidified atmosphere containing 5% CO₂, cells were washed once with the assay buffer (137 mM NaCl, 5 mM KCl, 1 mM CaCl₂, 1 mM MgCl₂, 50 mM Hepes, and 20 mM Tris, pH 7.4) and incubated with 250 μl of the buffer containing test compounds for 10 min at 37° C. Test compounds were dissolved in DMSO. The final concentration of DMSO was 0.5%. The transport reaction was initiated by addition of 50 μl [¹⁴C]-methyl-α-D-glucopyranoside (¹⁴C-AMG) solution (final concentration, 0.5 mM). After incubation for 2 hours at 37° C., the uptake was stopped by aspiration of the incubation mixture, the cells were washed three times with ice-cold PBS. Then, cells were solubilized with 0.3 N NaOH and aliquots were taken for determination of radioactivity by a liquid scintillation counter. Nonspecific AMG uptake was defined as that which occurred in the presence of 100 μM of phlorizin, a specific inhibitor of sodium-dependent glucose cotransporter. Specific uptake was normalized for the protein concentrations measured by the method of Bradford. The 50% inhibitory concentration (IC₅₀) values were calculated from dose-response curves by least square method.

### Results:

Results are shown in the following table:

TABLE 4

| Test Compounds | IC₅₀ |
|---|---|
| (Example No.) | (nM) |
| 1 | 180 |
| 2 | 21 |
| 5 | 5.3 |
| 6 | 31 |
| 8 | 41 |
| 10 | 22 |
| 13 | 110 |
| 14 | 97 |
| 15 | 15 |
| 16 | 130 |
| 17 | 63 |
| 18 | 190 |
| 19 | 46 |
| 20 | 180 |
| 22 | 210 |
| 23 | 170 |
| 25 | 67 |
| 26 | 190 |
| 28 | 19 |
| 30 | 54 |
| 31 | 150 |
| 33 | 10 |
| 34 | 35 |
| 35 | 17 |
| 36 | 120 |

### 2. Urinary Glucose Excretion Test in Rats

### Test Compounds:

Compounds described in the above examples were used for the Urinary glucose excretion test in rats.

### Methods:

6-week-old male Sprague-Dawley (SD) rats were housed in individual metabolic cages with free access to food and water from 2 days prior to the experiment. On the morning of the experiment, rats were administered vehicle (0.2% carboxymethyl cellulose solution containing 0.2% Tween80) or test compounds (30 mg/kg) by oral gavage at a volume of 10 ml/kg. Then, urine of the rat was collected for 24 hours, and the urine volume was measured. Subsequently, the glucose concentration in urine was quantified using the enzymatic assay kit and the daily amount of glucose excreted in urine per individual was calculated.

### Results:

Urinary glucose amounts ranges are depicted by A and B. These ranges are as follows: A≧2400 mg; 2400 mg>B≧2000 mg.

TABLE 5

| Test compounds | |
|---|---|
| (Example No.) | Urinary glucose |
| 2 | B |
| 5 | A |

1. A compound of formula (A), or a pharmaceutically acceptable salt thereof: 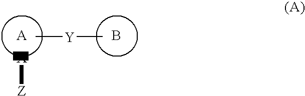
wherein Ring A and Ring B are independently an optionally substituted unsaturated heteromonocyclic ring, an optionally substituted unsaturated fused heterobicyclic ring, or an optionally substituted benzene ring;
X is carbon or nitrogen;
Y is —(CH₂)ₙ— (wherein n is 1 or 2); and
Z is one of the following groups: 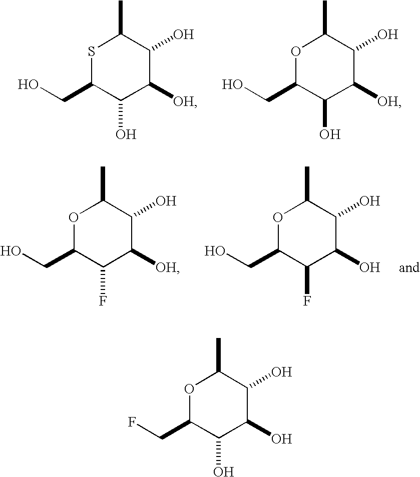
provided that:
(i) when Ring A is an optionally substituted unsaturated fused heterobicyclic ring, Y connects to the X-containing ring of said unsaturated fused heterobicyclic ring;
(ii) when Ring A is an optionally substituted unsaturated fused heterobicyclic ring, Z is: 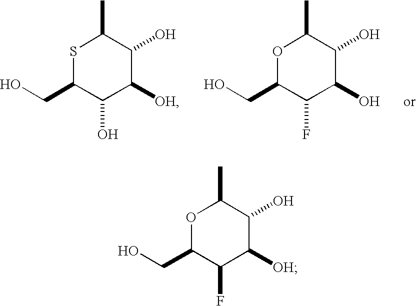
(iii) when both Ring A and Ring B are optionally substituted benzene, Z is:

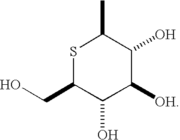

2. The compound according to claim 1, wherein Ring A is an optionally substituted unsaturated heteromonocyclic ring, Z is:

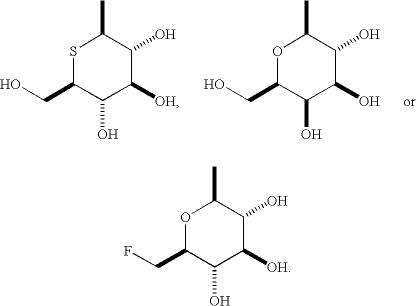

3. The compound according to claim 1, wherein:
(1) Ring A is an unsaturated heteromonocyclic ring which may optionally be substituted with 1-3 substituents independently selected from the group consisting of: halogen, hydroxy, cyano, nitro, alkyl, alkenyl, alkynyl, cycloalkyl, alkoxy, alkanoyl, alkylthio, alkylsulfonyl, alkylsulfinyl, amino, mono- or di-alkylamino, sulfamoyl, mono- or di-alkylsulfamoyl, carboxyl, alkoxycarbonyl, carbamoyl, mono- or di-alkylcarbamoyl, alkylsulfonylamino, phenyl, phenoxy, phenylsulfonylamino, phenylsulfonyl, heterocyclyl, and oxo, and
Ring B is an unsaturated heteromonocyclic ring, an unsaturated fused heterobicyclic ring, or a benzene ring, each of which may optionally be substituted with 1-3 substituents, independently selected from the group consisting of: halogen, hydroxy, cyano, nitro, alkyl, alkenyl, alkynyl, cycloalkyl, alkoxy, alkanoyl, alkylthio, alkylsulfonyl, alkylsulfinyl, amino, mono- or di-alkylamino, sulfamoyl, mono- or di-alkylsulfamoyl, carboxyl, alkoxycarbonyl, carbamoyl, mono- or di-alkylcarbamoyl, alkylsulfonylamino, phenyl, phenoxy, phenylsulfonylamino, phenylsulfonyl, heterocyclyl, alkylene, and alkenylene;
wherein each of the above-mentioned substituents on Ring A and Ring B may optionally be substituted with 1-3 substituents, independently selected from the group consisting of: halogen, cyano, alkyl, haloalkyl, alkoxy, haloalkoxy, alkanoyl, mono- or di-alkylamino, carboxyl, hydroxy, phenyl, alkylenedioxy, alkyleneoxy, alkoxycarbonyl, carbamoyl and mono- or di-alkylcarbamoyl;
(2) Ring A is a benzene ring which may optionally be substituted with 1-3 substituents, independently selected from the group consisting of: halogen, hydroxy, cyano, nitro, alkyl, alkenyl, alkynyl, cycloalkyl, alkoxy, alkanoyl, alkylthio, alkylsulfonyl, alkylsulfinyl, amino, mono- or di-alkylamino, alkanoylamino, sulfamoyl, mono- or di-alkylsulfamoyl, carboxyl, alkoxycarbonyl, carbamoyl, mono- or di-alkylcarbamoyl, alkylsulfonylamino, phenyl, phenoxy, phenylsulfonylamino, phenylsulfonyl, heterocyclyl, alkylene, and alkenylene, and
Ring B is an unsaturated heteromonocyclic ring or an unsaturated fused heterobicyclic ring, each of which may optionally be substituted with 1-3 substituents, independently selected from the group consisting of: halogen, hydroxy, cyano, nitro, alkyl, alkenyl, alkynyl, cycloalkyl, alkoxy, alkanoyl, alkylthio, alkylsulfonyl, alkylsulfinyl, amino, mono- or di-alkylamino, sulfamoyl, mono- or di-alkylsulfamoyl, carboxyl, alkoxycarbonyl, carbamoyl, mono- or di-alkylcarbamoyl, alkylsulfonylamino, phenyl, phenoxy, phenylsulfonylamino, phenylsulfonyl, heterocyclyl, alkylene and oxo;
wherein each of the above-mentioned substituents on Ring A and Ring B may optionally be substituted with 1-3 substituents, independently selected from the group consisting of: halogen, cyano, alkyl, haloalkyl, alkoxy, haloalkoxy, alkanoyl, mono- or di-alkylamino, carboxyl, hydroxy, phenyl, alkylenedioxy, alkyleneoxy, alkoxycarbonyl, carbamoyl and mono- or di-alkylcarbamoyl; or
(3) Ring A is an unsaturated fused heterobicyclic ring which may optionally be substituted with 1-3 substituents, independently selected from the group consisting of: halogen, hydroxy, cyano, nitro, alkyl, alkenyl, alkynyl, cycloalkyl, alkoxy, alkanoyl, alkylthio, alkylsulfonyl, alkylsulfinyl, amino, mono- or di-alkylamino, sulfamoyl, mono- or di-alkylsulfamoyl, carboxyl, alkoxycarbonyl, carbamoyl, mono- or di-alkylcarbamoyl, alkylsulfonylamino, phenyl, phenoxy, phenylsulfonylamino, phenylsulfonyl, heterocyclyl, and oxo, and
Ring B is an unsaturated heteromonocyclic ring, an unsaturated fused heterobicyclic ring, or a benzene ring, each of which may optionally be substituted with 1-3 substituents, independently selected from the group consisting of: halogen, hydroxy, cyano, nitro, alkyl, alkenyl, alkynyl, cycloalkyl, alkoxy, alkanoyl, alkylthio, alkylsulfonyl, alkylsulfinyl, amino, mono- or di-alkylamino, sulfamoyl, mono- or di-alkylsulfamoyl, carboxyl, alkoxycarbonyl, carbamoyl, mono- or di-alkylcarbamoyl, alkylsulfonylamino, phenyl, phenoxy, phenylsulfonylamino, phenylsulfonyl, heterocyclyl, alkylene and oxo;
wherein each of the above-mentioned substituents on Ring A and Ring B may optionally be substituted with 1-3 substituents, independently selected from the group consisting of: halogen, cyano, alkyl, haloalkyl, alkoxy, haloalkoxy, alkanoyl, mono- or di-alkylamino, carboxyl, hydroxy, phenyl, alkylenedioxy, alkyleneoxy, alkoxycarbonyl, carbamoyl and mono- or di-alkylcarbamoyl.

4. The compound according to claim 1, wherein:
(1) Ring A is an unsaturated heteromonocyclic ring which may optionally be substituted with halogen, lower alkyl, halo-lower alkyl, lower alkoxy, or oxo, and Ring B is (a) a benzene ring which may optionally be substituted with a group selected from: halogen; cyano; lower alkyl; halo-lower alkyl; lower alkoxy; halo-lower alkoxy; mono- or di-lower alkylamino; phenyl optionally substituted with halogen, cyano, lower alkyl, halo-lower alkyl, lower alkoxy, or mono- or di-lower alkylamino; and heterocyclyl optionally substituted with halogen, cyano, lower alkyl, halo-lower alkyl, lower alkoxy, or mono- or di-lower alkylamino; (b) an unsaturated heteromonocyclic ring which may optionally be substituted with a group selected from: halogen; cyano; lower alkyl; halo-lower alkyl; lower alkoxy; halo-lower alkoxy; mono- or di-lower alkylamino; phenyl optionally substituted with halogen, cyano, lower alkyl, halo-lower alkyl, lower alkoxy, or mono- or di-lower alkylamino; and heterocyclyl optionally substituted with halogen, cyano, lower alkyl, halo-lower alkyl, lower alkoxy, or mono- or di-lower alkylamino; or (c) an unsaturated fused heterobicyclic ring which may optionally be substituted with a group selected from: halogen; cyano; lower alkyl; halo-lower alkyl; lower alkoxy; halo-lower alkoxy; mono- or di-lower alkylamino; phenyl optionally substituted with halogen, cyano, lower alkyl, halo-lower alkyl, lower alkoxy, or mono- or di-lower alkylamino; and heterocyclyl optionally substituted with halogen, cyano, lower alkyl, halo-lower alkyl, lower alkoxy, or mono- or di-lower alkylamino;
(2) Ring A is a benzene ring which may optionally be substituted with halogen, lower alkyl, halo-lower alkyl, lower alkoxy, phenyl, or lower alkenylene, and Ring B is (a) an unsaturated heteromonocyclic ring which may optionally be substituted with a group selected from: halogen; cyano; lower alkyl; halo-lower alkyl; phenyl-lower alkyl; lower alkoxy; halo-lower alkoxy; mono- or di-lower alkylamino; phenyl optionally substituted with halogen, cyano, lower alkyl, halo-lower alkyl, lower alkoxy, halo-lower alkoxy, mono- or di-lower alkylamino, carbamoyl, or mono- or di-lower alkylcarbamoyl, and heterocyclyl optionally substituted with halogen, cyano, lower alkyl, halo-lower alkyl, lower alkoxy, halo-lower alkoxy, mono- or di-lower alkylamino, carbamoyl or mono- or di-lower alkylcarbamoyl; (b) an unsaturated fused heterobicyclic ring which may optionally be substituted with a group selected from: halogen; cyano; lower alkyl; halo-lower alkyl; phenyl-lower alkyl; lower alkoxy; halo-lower alkoxy; mo- or di-lower alkylamino; phenyl optionally substituted with halogen, cyano, lower alkyl, halo-lower alkyl, lower alkoxy, or mono- or di-lower alkylamino; and heterocyclyl optionally substituted with halogen, cyano, lower alkyl, halo-lower alkyl, lower alkoxy, or mono- or di-lower alkylamino; or
(3) Ring A is an unsaturated fused heterobicyclic ring which may optionally be substituted with halogen, lower alkyl, halo-lower alkyl, lower alkoxy, or oxo, and Ring B is (a) a benzene ring which may optionally be substituted with a group selected from: halogen; cyano; lower alkyl; halo-lower alkyl; lower alkoxy; halo-lower alkoxy; mo- or di-lower alkylamino; phenyl optionally substituted with halogen, cyano, lower alkyl, halo-lower alkyl, lower alkoxy, or mono- or di-lower alkylamino; and heterocyclyl optionally substituted with halogen, cyano, lower alkyl, halo-lower alkyl, lower alkoxy, or mono- or di-lower alkylamino; (b) an unsaturated heteromonocyclic ring which may optionally be substituted with a group selected from: halogen; cyano; lower alkyl; halo-lower alkyl; lower alkoxy; halo-lower alkoxy; mono- or di-lower alkylamino; phenyl optionally substituted with halogen, cyano, lower alkyl, halo-lower alkyl, lower alkoxy, or mono- or di-lower alkylamino; and heterocyclyl optionally substituted with halogen, cyano, lower alkyl, halo-lower alkyl, lower alkoxy, or mono- or di-lower alkylamino; or (c) an unsaturated fused heterobicyclic ring which may optionally be substituted with a group selected from: halogen; cyano; lower alkyl; halo-lower alkyl; lower alkoxy; halo-lower alkoxy; mo- or di-lower alkylamino; phenyl optionally substituted with halogen, cyano, lower alkyl, halo-lower alkyl, lower alkoxy, or mono- or di-lower alkylamino; and heterocyclyl optionally substituted with halogen, cyano, lower alkyl, halo-lower alkyl, lower alkoxy, or mono- or di-lower alkylamino.

5. The compound according to claim 1, wherein Y is —CH₂— and is linked at the 3-position of Ring A, with respect to X being the 1-position.

6. The compound according to claim 1, wherein:
(1) Ring A is a benzene ring which may optionally be substituted with 1-3 substituents, independently selected from the group consisting of: halogen, lower alkyl optionally substituted with halogen or lower alkoxy, lower alkoxy optionally substituted with halogen or lower alkoxy, cycloalkyl, cycloalkoxy, phenyl, and lower alkenylene, and Ring B is an unsaturated heteromonocyclic ring or an unsaturated fused heterobicyclic ring, each of which may optionally be substituted with 1-3 substituents, independently selected from the group consisting of: halogen; lower alkyl optionally substituted with halogen, lower alkoxy or phenyl; lower alkoxy optionally substituted with halogen or lower alkoxy; cycloalkyl; cycloalkoxy; phenyl optionally substituted with halogen, cyano, lower alkyl, halo-lower alkyl, lower alkoxy, halo-lower alkoxy, mono- or di-lower alkylamino, carbamoyl or mono- or di-lower alkylcarbamoyl; heterocyclyl optionally substituted with halogen, cyano, lower alkyl, halo-lower alkyl, lower alkoxy, halo-lower alkoxy, mono- or di-lower alkylamino, carbamoyl or mono- or di-lower alkylcarbamoyl; and oxo;
(2) Ring A is an unsaturated heteromonocyclic ring which may optionally be substituted with 1-3 substituents, independently selected from the group consisting of: halogen, lower alkyl optionally substituted with lower alkoxy, lower alkoxy optionally substituted with halogen or lower alkoxy, cycloalkyl, cycloalkoxy, and oxo, and Ring B is a benzene ring which may optionally be substituted with 1-3 substituents, independently selected from the group consisting of: halogen; lower alkyl optionally substituted with halogen, lower alkoxy or phenyl; lower alkoxy optionally substituted with halogen or lower alkoxy; cycloalkyl; cycloalkoxy; phenyl optionally substituted with halogen, cyano, lower alkyl, halo-lower alkyl, lower alkoxy or halo-lower alkoxy; heterocyclyl optionally substituted with halogen, cyano, lower alkyl, halo-lower alkyl, lower alkoxy or halo-lower alkoxy; and lower alkylene,
(3) Ring A is an unsaturated heteromonocyclic ring which may optionally be substituted with 1-3 substituents, independently selected from the group consisting of: halogen, lower alkyl optionally substituted with halogen or lower alkoxy, lower alkoxy optionally substituted with halogen or lower alkoxy, cycloalkyl, cycloalkoxy, and oxo, Ring B is an unsaturated heteromonocyclic ring or an unsaturated fused heterobicyclic ring, each of which may optionally be substituted with 1-3 substituents, independently selected from the group consisting of: halogen; lower alkyl optionally substituted with halogen, lower alkoxy or phenyl; lower alkoxy optionally substituted with halogen or lower alkoxy; cycloalkyl; cycloalkoxy; phenyl optionally substituted with halogen, cyano, lower alkyl, halo-lower alkyl, lower alkoxy or halo-lower alkoxy; heterocyclyl optionally substituted with halogen, cyano, lower alkyl, halo-lower alkyl, lower alkoxy or halo-lower alkoxy; and oxo;
(4) Ring A is an unsaturated fused heterobicyclic ring which may optionally be substituted with 1-3 substituents, independently selected from the group consisting of: halogen, lower alkyl optionally substituted with halogen or lower alkoxy, lower alkoxy optionally substituted with halogen or lower alkoxy, cycloalkyl, cycloalkoxy, and oxo, Ring B is a benzene ring or an unsaturated heteromonocyclic ring, each of which may optionally be substituted with 1-3 substituents, independently selected from the group consisting of: halogen; lower alkyl optionally substituted with halogen, lower alkoxy or phenyl; lower alkoxy optionally substituted with halogen or lower alkoxy; cycloalkyl; cycloalkoxy; phenyl optionally substituted with halogen, cyano, lower alkyl, halo-lower alkyl, lower alkoxy or halo-lower alkoxy; heterocyclyl optionally substituted with halogen, cyano, lower alkyl, halo-lower alkyl, lower alkoxy or halo-lower alkoxy; and lower alkylene, or
(5) Ring A is an unsaturated heteromonocyclic ring which may optionally be substituted with 1-3 substituents, independently selected from the group consisting of: halogen, lower alkyl optionally substituted with lower alkoxy, lower alkoxy optionally substituted with halogen or lower alkoxy, cycloalkyl, cycloalkoxy, and oxo, Ring B is an unsaturated heteromonocyclic ring or an unsaturated fused heterobicyclic ring, each of which may optionally be substituted with 1-3 substituents, independently selected from the group consisting of: halogen; lower alkyl optionally substituted with halogen, lower alkoxy or phenyl; lower alkoxy optionally substituted with halogen or lower alkoxy; cycloalkyl; cycloalkoxy; phenyl optionally substituted with halogen, cyano, lower alkyl, halo-lower alkyl, lower alkoxy or halo-lower alkoxy; heterocyclyl optionally substituted with halogen, cyano, lower alkyl, halo-lower alkyl, lower alkoxy or halo-lower alkoxy; and oxo.

7. The compound according to claim 1, wherein Ring A is 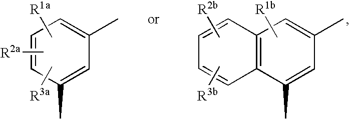
wherein R^{1a}, R^{2a}, R^{3a}, R^{1b}, R^{2b}, and R^{3b} are each independently hydrogen, halogen, hydroxy, alkoxy, alkyl, haloalkyl, haloalkoxy, hydroxyalkyl, alkoxyalkyl, alkoxyalkoxy, alkenyl, alkynyl, cycloalkyl, cycloalkenyl, cycloalkyloxy, phenyl, phenylalkoxy, cyano, nitro, amino, mono- or di-alkylamino, alkanoylamino, carboxyl, alkoxycarbonyl, carbamoyl, mono- or di-alkylcarbamoyl, alkanoyl, alkylsulfonylamino, phenylsulfonylamino, alkylsulfinyl, alkylsulfonyl, or phenylsulfonyl; Ring B is 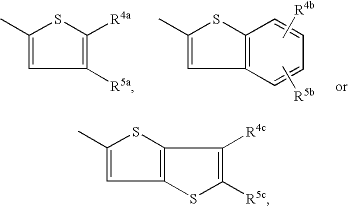
wherein R^{4a} and R^{5a} are each independently hydrogen; halogen; hydroxy; alkoxy; alkyl; haloalkyl; haloalkoxy; hydroxyalkyl; alkoxyalkyl; phenylalkyl; alkoxyalkoxy; hydroxyalkoxy; alkenyl; alkynyl; cycloalkyl; cycloalkenyl; cycloalkyloxy; phenyloxy; phenylalkoxy; cyano; nitro; amino; mono- or di-alkylamino; alkanoylamino; carboxyl; alkoxycarbonyl; carbamoyl; mono- or di-alkylcarbamoyl; alkanoyl; alkylsulfonylamino; phenylsulfonylamino; alkylsulfinyl; alkylsulfonyl; phenylsulfonyl; phenyl optionally substituted with 1-3 groups selected from halogen, cyano, alkyl, haloalkyl, alkoxy, haloalkoxy, alkylenedioxy, alkyleneoxy, mono- or di-alkylamino, carbamoyl, and mono- or di-alkylcarbamoyl; or heterocyclyl optionally substituted with 1-3 groups selected from halogen, cyano, alkyl, haloalkyl, alkoxy, haloalkoxy, mono- or di-alkylamino, carbamoyl, and mono- or di-alkylcarbamoyl, or R^{4a} and R^{5a} are bonded to each other at the terminals thereof to form alkylene;
R^{4b}, R^{5b}, R^{4c} and R^{5c} are each independently hydrogen; halogen; hydroxy; alkoxy; alkyl; haloalkyl; haloalkoxy; hydroxyalkyl; alkoxyalkyl; phenylalkyl; alkoxyalkoxy; hydroxyalkoxy; alkenyl; alkynyl; cycloalkyl; cycloalkenyl; cycloalkyloxy; phenyloxy; phenylalkoxy; cyano; nitro; amino; mono- or di-alkylamino; alkanoylamino; carboxyl; alkoxycarbonyl; carbamoyl; mono- or di-alkylcarbamoyl; alkanoyl; alkylsulfonylamino; phenylsulfonylamino; alkylsulfinyl; alkylsulfonyl; phenylsulfonyl; phenyl optionally substituted with 1-3 groups selected from: halogen, cyano, alkyl, haloalkyl, alkoxy, haloalkoxy, alkylenedioxy, alkyleneoxy, and mono- or di-alkylamino; or heterocyclyl optionally substituted with 1-3 groups selected from: halogen, cyano, alkyl, haloalkyl, alkoxy and haloalkoxy; and
Z is:

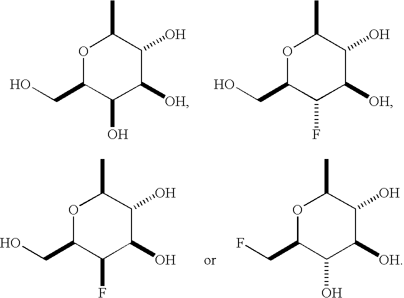

8. The compound according to claim 7, wherein R^{1a}, R^{2a}, R^{3a}, R^{1b}, R^{2b}, and R^{3b} are each independently hydrogen, halogen, lower alkyl, halo-lower alkyl, lower alkoxy, or phenyl;
R^{4a} and R^{5a} are each independently hydrogen; halogen; lower alkyl; halo-lower alkyl; phenyl-lower alkyl; phenyl optionally substituted with 1-3 groups selected from: halogen, cyano, lower alkyl, halo-lower alkyl, lower alkoxy, halo-lower alkoxy, methylenedioxy, ethyleneoxy, mono- or di-lower alkylamino, carbamoyl, and mono- or di-lower alkylcarbamoyl; or heterocyclyl optionally substituted with 1-3 groups selected from: halogen, cyano, lower alkyl, halo-lower alkyl, lower alkoxy, halo-lower alkoxy, mono- or di-alkylamino, carbamoyl, and mono- or di-lower alkylcarbamoyl, or R^{4a} and R^{5a} are bonded to each other at the terminals thereof to form lower alkylene; and
R^{4b}, R^{5b}, R^{4c} and R^{5c} are each independently hydrogen, halogen, lower alkyl, halo-lower alkyl, lower alkoxy, or halo-lower alkoxy.

9. The compound according to claim 7, wherein Ring A is 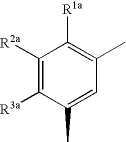
in which R^{1a} is halogen, lower alkyl, or lower alkoxy, and R^{2a} and R^{3a} are hydrogen; Ring B is 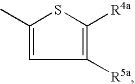
in which R^{4a} is phenyl optionally substituted with 1-3 groups selected from halogen, cyano, lower alkyl, halo-lower alkyl, lower alkoxy, halo-lower alkoxy, methylenedioxy, ethyleneoxy, mono- or di-lower alkylamino, carbamoyl, and mono- or di-lower alkylcarbamoyl; or heterocyclyl optionally substituted with 1-3 groups selected from halogen, cyano, lower alkyl, lower alkoxy, mono- or di-alkylamino, carbamoyl, and mono- or di-lower alkylcarbamoyl, and R^{5a} is hydrogen; or R^{4a} and R^{5a} are bonded to each other at the terminals thereof to form lower alkylene; and Y is —CH₂—.

10. The compound according to claim 9, wherein R^{1a} is halogen or lower alkyl; R^{4a} is phenyl optionally substituted with 1-3 groups selected from halogen, cyano, lower alkyl, halo-lower alkyl, lower alkoxy, halo-lower alkoxy, mono- or di-lower alkylamino, carbamoyl, and mono- or di-lower alkylcarbamoyl; or heterocyclyl optionally substituted with 1-3 groups selected from: halogen, cyano, lower alkyl, lower alkoxy, carbamoyl, and mono- or di-lower alkylcarbamoyl; and R^{5a} is hydrogen.

11. The compound according to claim 10, wherein R^{4a} is phenyl optionally substituted with halogen, cyano, lower alkyl, halo-lower alkyl, lower alkoxy, halo-lower alkoxy, or mono- or di-lower alkylamino; or heterocyclyl optionally substituted with halogen, cyano, lower alkyl, halo-lower alkyl, lower alkoxy, or halo-lower alkoxy.

12. The compound according to claim 11, wherein R^{4a} is phenyl substituted with halogen, cyano, lower alkyl, halo-lower alkyl, lower alkoxy or halo-lower alkoxy; or heterocyclyl substituted with halogen, cyano, lower alkyl, or lower alkoxy.

13. The compound according to claim 12, wherein R^{4a} is phenyl substituted with halogen or cyano, or pyridyl substituted with halogen.

14. The compound according to claim 7, wherein Ring A is 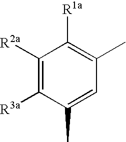
in which R^{1a} is halogen, lower alkyl, or lower alkoxy, and R^{2a} and R^{3a} are both hydrogen; and Ring B is 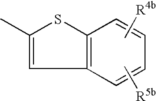
in which R^{4b} and R^{5b} are each independently hydrogen, halogen, lower alkyl, halo-lower alkyl, lower alkoxy, or halo-lower alkoxy.

15. The compound according to claim 1, wherein Ring A is: 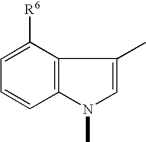
in which R⁶ is hydrogen, halogen, or alkyl; Ring B is: 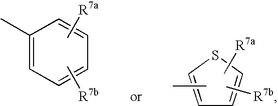
in which R^{7a} and R^{7b} are independently hydrogen, halogen, alkyl, cycloalkyl, haloalkyl, alkoxy, haloalkoxy, alkylthio, hydroxy, phenyl, halophenyl, cyanophenyl, pyridyl, halopyridyl, thienyl, or halothienyl, or R^{7a} and R^{7b} together with carbon atoms to which they are attached form a fused benzene, furan or dihydrofuran ring; and Y is CH₂.

16. The compound according to claim 15, wherein R⁶ is halogen, Z is 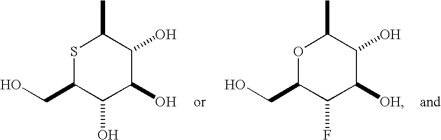
R^{7a} and R^{7b} are independently hydrogen, halogen, alkyl, cycloalkyl, haloalkyl, alkoxy, haloalkoxy, alkylthio, phenyl, halophenyl, cyanophenyl, pyridyl or halopyridyl, or R^{7a} and R^{7b} together with carbon atoms to which they are attached form a fused benzene, furan or dihydrofuran ring.

17. The compound according to claim 16, wherein R^{7a} and R^{7b} are independently hydrogen, halogen, alkyl, cycloalkyl, haloalkyl, alkoxy, haloalkoxy, or alkylthio, or R^{7a} and R^{7b} together with carbon atoms to which they are attached form a fused furan or dihydrofuran ring.

18. The compound according to claim 17, wherein R^{7a} and R^{7b} are independently hydrogen, halogen, alkyl, cycloalkyl, haloalkyl, alkoxy, or haloalkoxy, or R^{7a} and R^{7b} together with carbon atoms to which they are attached form a fused furan or dihydrofuran ring.

19. The compound according to claim 16, R⁶ is halogen, Ring B is: 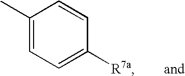
R^{7a} is halogen, alkyl, cycloalkyl, haloalkyl, alkoxy, haloalkoxy or alkylthio.

20. The compound according to claim 19, wherein R^{7a} is halogen, alkyl, cycloalkyl, or alkoxy.

21. The compound according to claim 1, wherein the compound is selected from:
3-(4-Ethylphenylmethyl)-1-(5-thio-β-D-glucopyranosyl)indole,
4-Chloro-3-(4-ethylphenylmethyl)-1-(5-thio-β-D-glucopyranosyl)indole,
4-Chloro-3-(4-ethylphenylmethyl)-1-(4-fluoro-4-deoxy-β-D-glucopyranosyl)indole,
4-Chloro-3-(4-ethoxyphenylmethyl)-1-(5-thio-β-D-glucopyranosyl)indole,
3-(Benzo[b]thiophen-2-ylmethyl)-4-chloro-1-(4-fluoro-4-deoxy-β-D-galactopyranosyl)benzene,
4-Chloro-3-(5-phenyl-2-thienylmethyl)-1-(6-fluoro-6-deoxy-β-D-glucopyranosyl)benzene, and
4-Chloro-3-(5-phenyl-2-thienylmethyl)-1-(4-fluoro-4-deoxy-β-D-glucopyranosyl)benzene;
or a pharmaceutically acceptable salt thereof.

22. A pharmaceutical composition comprising the compound as set forth in claim 1 and a pharmaceutically acceptable carrier or diluent.

23. The pharmaceutical composition according to claim 22, which further comprises another antidiabetic agent.

24. A compound as set forth in claim 1 for use as an active therapeutic substance.

25. Use of a compound as set forth in claim 1 in the manufacture of a medicament for use in the treatment of disorders selected from diabetes mellitus, diabetic retinopathy, diabetic neuropathy, diabetic nephropathy, delayed wound healing, insulin resistance, hyperglycemia, hyperinsulinemia, elevated blood levels of fatty acids, elevated blood levels of glycerol, hyperlipidemia, obesity, hypertriglyceridemia, Syndrome X, diabetic complications, atherosclerosis, and hypertension.

26. A method for treatment or delaying the progression or onset of diabetes mellitus, diabetic retinopathy, diabetic neuropathy, diabetic nephropathy, delayed wound healing, insulin resistance, hyperglycemia, hyperinsulinemia, elevated blood levels of fatty acids, elevated blood levels of glycerol, hyperlipidemia, obesity, hypertriglyceridemia, Syndrome X, diabetic complications, atherosclerosis, or hypertension, which comprises administering to a mammalian species in need of treatment a therapeutically effective amount of the compound as set forth in claim 1.

27. A method for treatment of type 1 or type 2 diabetes mellitus, which comprises administering to a mammalian species in need of treatment a therapeutically effective amount of the compound as set forth in claim 1 alone, or in combination with another antidiabetic agent, an agent for treating diabetic complications, an anti-obesity agent, an antihypertensive agent, an antiplatelet agent, an anti-atherosclerotic agent and/or a hypolipidemic agent.
